(19) United States
(12) Patent Application Publication
(10) Pub. No.: US 2007/0202561 A1
(43) Pub. Date: Aug. 30, 2007

(54) Electronic Detection Immunoassays that Utilize a Binder Support Medium

(75) Inventor: Robert W. Rosenstein, Ellicott City, MD (US)

Correspondence Address: DAVID W. HIGHET, VP AND CHIEF IP COUNSEL;BECTON, DICKINSON AND COMPANY, 1 BECTON DRIVE, MC 110, FRANKLIN LAKES, NJ, 07417-1880, US

(73) Assignee: BECTON DICKINSON AND COMPANY, Franklin Lakes, NJ (US)

(21) Appl. No.: 11/463,909

(22) Filed: Aug. 11, 2006

Related U.S. Application Data
(60) Provisional application No. 60/772,268, filed on Feb. 10, 2006.

(51) Int. Cl. C12Q 1/54; C12M 3/00

(52) U.S. Cl. 435/14; 435/287.1

(57) ABSTRACT

A binder support medium-based immunoassay device and method is provided, utilizing the catalyzed formation of dopants and their subsequent effects on electroconductive polymers to detect an analyte of interest.

### FIELD OF THE INVENTION

The invention relates to immunoassays that detect the presence or amount of a particular analyte.

### BACKGROUND OF THE INVENTION

In vitro diagnostic (IVD) tests have revolutionized the rapid analysis of analytes, and allow for a simple and cost effective detection method for a myriad of moieties including proteins such as enzymes and hormones, drugs and drug metabolites, antibodies, and nucleic acids. Many of these tests are based on immunoassays that combine the principles of chemistry and immunology to provide for quantitative and qualitative analyses of target analytes. The basic principle of these assays is the detection of an analyte-receptor reaction.

In recent years, immunoassays have evolved from expensive and complex procedures requiring calibrated machinery and skilled technicians for operation to simpler designs such as dip-sticks and test strips, which use relatively inexpensive binder support mediums and are easily operated by anyone because they only require the user to follow a simple series of directions. Today, immunoassays based on these binder support mediums offer rapid results and increasing sensitivity for the detection of a large number of analytes of interest. For example, the in-home use of immunoassay pregnancy tests that qualitatively detect the presence of human chorionic gonadotropin (hCG) in the urine is commonplace.

Although there are many permutations to the design of the binder support medium-based immunoassay, the basic design involves detecting the analyte of interest following its binding to a labeled receptor (or “tracer”), and the necessary separation of the free labeled receptor from the bound labeled receptor. The analyte of interest is generally contained or placed in a liquid sample that is then added to the immunoassay. As the liquid sample interacts with the active reagents in the immunoassay, immunological or chemical reactions may occur that ultimately allow for the detection of the presence of the analyte of interest.

The basic design of the binder support medium based immunoassay comprises the flow of a liquid containing or lacking an analyte of interest across or through a porous membrane or series of porous membranes, at least one of which acts as a binder support medium. The liquid generally flows due to capillary action or capillary flow, which is essentially the process by which water is drawn from a wet area in a medium and transported to a dry area through the pores of a material. Capillary action or flow is caused by capillary forces acting on the liquid such as adhesion, cohesion, and surface tension. Alternatively, the liquid may flow due to gravity.

There are generally two types of assay formats utilized in the binder support medium devices, sandwich type assays and competitive type assays. In a “sandwich” type assay, as the liquid sample flows across or through the device, the analyte, if present, binds with a receptor capable of detection, which is usually an antibody that recognizes the analyte of interest and is bound to a label (or “tracer”) moiety that can be detected, either by the operator or by a machine. The labeled receptor is generally located within the membrane or one of the membranes of the immunoassay device. As the analyte-labeled receptor complex flows across or through the membrane or membranes via capillary action or gravity, it eventually comes into contact with a detection zone on the binder support medium of the immunoassay that comprises an immobilized capture receptor ligand known as a binder, where it is bound thereby forming a analyte-labeled receptor complex-binder “sandwich”. The presence or absence of the analyte of interest is determined through inspection of the detection zone, where the presence of the analyte is indicated usually by a specific visually detectable signal.

In a competitive type assay, the labeled receptor may be bound to a competitor, which is generally the analyte itself, an analogue or derivative thereof, or a moiety that is incapable of binding to the analyte. The competitor is capable of competing with the analyte for an immobilized binder in the detection zone located in the binder support medium. As the liquid sample flows into the detection zone, the analyte contained in the liquid sample competes with the competitor containing the labeled receptor in binding the immobilized binder. Generally, the greater the amounts of analyte present in the liquid sample, the lesser the amount of competitor bound in the detection zone.

The reliability of the detection of the analyte is a critical component of any immunoassay device. The precision of the assay is largely dependent on the ability to detect the analyte through its interaction with a labeled receptor. Many immunoassay devices rely on a chemical or biochemical label or tracer that provides a colorimetric indication of the presence or absence of an analyte of interest. This label becomes concentrated in the detection zone when the analyte-labeled receptor complex becomes bound by an immobilized binder. There is an ongoing need for fast, reliable, sensitive, and economical detection schemes for use in immunoassay devices.

### SUMMARY OF THE INVENTION

In one embodiment, binder support medium-based immunoassay devices, methods, and kits are provided for the determination of the presence or amount of an analyte of interest in a sample that utilize an electrical circuit and a conducting polymer component to detect the generation of a dopant molecule or compound. The dopant is generated only in the presence of the analyte or a competitor, wherein the analyte or competitor is bound to a particular reagent necessary for the generation of the dopant. Changes in the electrical conductivity of the conducting polymer (typically an increase in the conductivity) are induced by the generation of the dopant, allowing for the detection of an electrical signal by a readout mechanism, indicating the presence, absence, or amount of the analyte.

In one embodiment, the analyte or competitor is bound to a reagent that directly participates in the generation of the dopant. The generation of the dopant occurs when the analyte or competitor bound to the necessary reagent becomes immobilized by a binder in a detection zone present on a binder support medium. In one embodiment, the analyte of interest is bound to the reagent via a labeled receptor interaction following application of the sample to the assay, wherein the label of the labeled receptor comprises the reagent. In an alternative embodiment, the reagent is bound to a competitor, the competitor being an analyte or analyte analogue that is contained in the assay prior to the application of the sample. The reagent bound to the analyte or competitor interacts with other reagents in the detection zone, producing a dopant that affects the electrical conductivity of a conducting polymer component. In certain embodiments, the bound reagent possesses catalytic activity, wherein the reagent is capable of catalyzing other reagents to form a dopant. In certain embodiments, the bound reagent is a reduction-oxidation catalyst. In a more particular embodiment, the bound reagent is a reduction-oxidation enzyme and the generated dopant is an iodine, iodide, or iodide derived molecule, such as tri-iodide. In a more particular embodiment, the bound reagent is lactose peroxidase or glucose oxidase, and the dopant generated is tri-iodide.

The amount of electrical current that is conducted by the conducting polymer component can be proportional to the amount of dopant generated in the assay, and thus, determinant of the presence or amount of analyte, depending on the type of assay utilized. Typically, the conducting polymer acts as a switch: when doped, the electrical current that passes through the conducting polymer component can be measured and displayed by an appropriate readout mechanism, providing the user with an indication of the presence or amount of analyte in the sample. The readout mechanism can be programmed to report the presence or absence of an analyte at various threshold levels, providing flexibility in determining the limits of detection. Because a readout mechanism provides the indication of the presence or amount of the analyte of interest, the present invention may reduce the errors associated with user interpretation.

In one embodiment, the present invention provides for a single reduction-oxidation enzyme system, wherein a reduction-oxidation enzyme is bound to an analyte or competitor and capable of generating a dopant in the presence of other reagents in a detection zone. In an alternative embodiment, the present invention provides for a two enzyme system, wherein a first reduction-oxidation enzyme or a second reduction-oxidation enzyme is bound to an analyte or competitor, and the first reduction-oxidation enzyme or second reduction-oxidation enzyme participates in a sequence of chemical interactions within a detection zone to generate a dopant.

The present invention is not limited to a particular type of assay, and can be utilized in sandwich-type assays, as well as competitive-type assays, and the modifications to the assays based on the type of assay utilized are generally known by one of ordinary skill in the art. In addition, the present invention is not limited to the detection of a single analyte, but can detect one or more than one analyte of interest. The present invention is also not limited to the type of flow assay utilized. For example, the present invention can be utilized in a lateral flow based immunoassay, vertical flow or flow through based immunoassay, or a combination device utilizing both lateral and vertical flow.

### BRIEF DESCRIPTION OF THE FIGURES

Embodiments of the invention will now be described by way of example only and with reference to the accompanying drawings.

FIG. 1 is a schematic of the immunoassay device according to one embodiment of the invention.

FIG. 2 is a schematic of a catalytic reduction oxidation reaction according to one embodiment of the invention.

FIG. 3 is a schematic of a catalytic reduction oxidation reaction according to one embodiment of the invention.

FIG. 4A is a schematic of a flow chart of events in the immunoassay process according to one embodiment of the invention.

FIG. 4B is a flow chart of events in the immunoassay process according to one embodiment of the invention.

FIGS. 5A-5E are schematics depicting chemical events of a one-enzyme sandwich assay according to one embodiment of the invention.

FIGS. 6A-6D are schematics depicting chemical events of a one enzyme competitive assay according to one embodiment of the invention.

FIG. 7 is a schematic of a catalytic reduction oxidation sequence according to one embodiment of the invention.

FIG. 8 is a schematic of a catalytic reduction oxidation sequence according to one embodiment of the invention.

FIG. 9A is a schematic of a flow chart of events in the immunoassay process according to one embodiment of the invention.

FIG. 9B is a flow chart of events in the immunoassay process according to one embodiment of the invention.

FIGS. 10A-10G are schematics depicting chemical events of a two-enzyme sandwich assay according to one embodiment of the invention.

FIGS. 11A-11E are schematics depicting chemical events of a two-enzyme sandwich assay according to one embodiment of the invention.

FIGS. 12A-12E are schematics depicting chemical events a two-enzyme competitive assay according to one embodiment of the invention.

FIGS. 13A-13E are schematics depicting chemical events of a two-enzyme competitive assay according to one embodiment of the invention.

FIG. 14 is a schematic of the immunoassay device according to one embodiment of the invention.

FIG. 15 is a schematic of the immunoassay device according to one embodiment of the invention.

FIG. 16 is a schematic of the immunoassay device according to one embodiment of the invention.

FIG. 17 is a schematic of the immunoassay device according to one embodiment of the invention.

FIG. 18 is a schematic of the immunoassay device according to one embodiment of the invention.

### DETAILED DESCRIPTION

The term “analyte” refers to the compound or composition to be detected. The analyte can be any substance for which there exists a technique for measuring utilizing a ligand-receptor interaction. Possible analytes include virtually any compound, composition, aggregation, or other substance that may be detected through a ligand-receptor interaction. For example, the analyte, or portion thereof, can be antigenic or haptenic having at least one determinant site, wherein a naturally occurring, or synthetically derived antibody binds thereto.

Analytes that can be analyzed utilizing the present invention include, but are not limited to, toxins, organic compounds, proteins, peptides, microorganisms, bacteria, viruses, amino acids, nucleic acids, carbohydrates, hormones, steroids, vitamins, drugs including those administered for therapeutic purposes as well as those administered for illicit purposes, pollutants, pesticides, and metabolites of or antibodies to any of the above substances. The term analyte also includes any antigenic substances, haptens, antibodies, macromolecules, and combinations thereof.

The term “sample” refers to anything which may contain an analyte for which an analyte assay is desired. The sample may be a biological sample, such as a biological fluid or a biological tissue. Examples of biological fluids include urine, blood, plasma, serum, saliva, semen, stool, sputum, cerebral spinal fluid, tears, mucus, amniotic fluid or the like. Biological tissues are aggregates of cells, usually of a particular kind together with their intercellular substance that form one of the structural materials of a human, animal, plant, bacterial, fungal or viral structure, including connective, epithelium, muscle and nerve tissues. Examples of biological tissues also include organs, tumors, lymph nodes, arteries and individual cell(s).

“Liquid sample” refers to a material suspected of containing the analyte(s) of interest, which material has sufficient fluidity to flow through a device in accordance herewith. The terms liquid sample and fluid sample are used interchangeably here. Fluid sample can be used as obtained directly from the source or following a pretreatment so as to modify its character. Such samples can include human, animal or man-made samples. The sample can be prepared in any convenient medium which does not interfere with the assay. Typically, the sample is an aqueous solution or biological fluid. In a particular embodiment, the liquid sample may also drive the binding reactions within the assay device by allowing the analyte to move first into the device, and then to sequentially bind to the labeled receptor and binder.

The liquid sample can be derived from any source, such as a physiological fluid, including blood, serum, plasma, saliva, sputum, ocular lens fluid, sweat, urine, milk, ascites fluid, mucous, synovial fluid, peritoneal fluid, transdermal exudates, pharyngeal exudates, bronchoalveolar lavage, tracheal aspirations, cerebrospinal fluid, semen, cervical mucus, vaginal or urethral secretions, amniotic fluid, and the like. Herein, fluid homogenates of cellular tissues such as, for example, hair, skin and nail scrapings, meat extracts and skins of fruits and nuts are also considered biological fluids.

The liquid sample can be treated prior to its application on the immunoassay device. Treatment, if necessary, may involve preparing plasma from blood, diluting viscous fluids, and the like. Methods of treatment can involve, but are not be limited to, filtration, distillation, separation, concentration, inactivation of interfering components, and the addition of reagents. Methods utilized for the selection and pretreatment of biological, industrial, and environmental samples prior to testing are generally known by one of ordinary skill in the art. Besides physiological fluids, other samples can be used such as water, food products, soil extracts, and the like for the performance of industrial, environmental, or food production assays as well as diagnostic assays. In addition, a solid material suspected of containing the analyte can be used as the test sample once it is modified to form a liquid medium or to release the analyte. The selection and pretreatment of biological, industrial, and environmental samples prior to testing is well known in the art.

The term “binder support medium” as used herein refers to substrate materials that have the functionality such as described herein. Examples include, but are not limited to, materials having capillarity and the capacity for chromatographic solvent transport of non-immobilized reagents and reactive sample components by means of a selected chromatographic solvent, that is, binder support media are suitably absorbent, porous or capillary possessing materials through which a solution containing an analyte can be transported by capillary action or gravity. Illustrative examples of binder support media include microporous or microgranular thin layer chromatography substrate comprising materials that are inert toward or generally physically or chemically unreactive with any of the reagents, labels, buffers, reaction products, or liquid sample's components. It is also possible to use substrates (e.g., plastic) having structures or features therein that provide the desired flow characteristics, e.g., plastic with molded-in microfluidics features.

Examples of granular thin layer chromatographic materials such as silica or microgranular cellulose that are suitable for binder support media according to the present invention include non-granular microporous materials, including microporous cellulose esters, e.g., esters of cellulose with an aliphatic carboxylic acid, such as an alkane carboxylic acid, having from 1 to 7 carbon atoms, e.g., acetic acid, propionic acid, or any of the butyric acids or valeric acids, can be provided. Other examples include microporous materials made from nitrocellulose, by which term any nitric acid ester of cellulose is intended, and these may include nitrocellulose in combination with any of the said carboxylic acid cellulose esters. Thus, pure nitrocellulose esters can be used as consisting of an ester of cellulose having approximately 3 nitric groups per 6 carbon atoms. A specific example is Type SMWP material (Millipore Corp., Bedford, Mass.) which has a pore size of 5 μm.

Binder support media useful for the present invention include natural, synthetic, or synthetically modified naturally occurring materials. These include cellulosic materials such as paper, cellulose, cotton cloth, and derivatives such as cellulose acetate and nitrocellulose. Other polysaccharide-based examples are starch-based materials such as Sephadex RTM brand cross-linked dextran chains, and polysaccharide gels such as agarose and dextran gels. Other illustrative binder support media include: synthetic cloth (e.g., nylon cloth); plastic films such as polyvinyl chloride optionally in combination with silica; glassy matrices (e.g., fiberglass or glass fiber filter paper); ceramic materials; porous gels such as gelatin or silica gel; and the like.

The terms “vertical” or “vertically” as used herein generally means parallel to the thickness or depth, as opposed to the length and width dimensions of the elements utilized in the device, such as the pads or mediums. The term “lateral” or “laterally” as used herein generally means parallel to the length, as opposed to the width and depth dimensions of the elements utilized in the device, such as the pads and mediums. In many embodiments, the elements are substantially planar and have a length, or lateral dimension, that is greater than the thickness, or vertical dimension. However, it is recognized that the magnitudes of these dimensions relative to each other can be changed without departing from the scope and spirit of the invention.

Generally, the terms “vertical,” “vertically,” “lateral,” and “laterally” also can describe the juxtaposition orientation of the elements of the device. For vertically juxtaposed elements, a line normal to and intersecting the planar surface of one such element also will be substantially normal to and intersect the planar surface of the other vertically juxtaposed elements. It is further recognized that gravity is not necessary for the operation of the device because the liquid sample may flow by capillary flow or action through the pads and mediums of the device.

For descriptions that refer to juxtaposition of one element of the device to another, it should be understood that the juxtaposition may be vertical, lateral, or otherwise adjacent juxtaposition. It should also be understood that the invention is not limited to per se juxtaposition of elements in any of the illustrated or described embodiments. For example, an element that can exist in fluid communication with another, such as a sample pad, tracer pad, reagent pad, sump, conducting polymer component, or binder support medium, may have interposed between it and another element an additional article that permits fluid communication including, but not limited to a spacer, filter pad, polymeric separator membrane, soluble composition layer, porous metal layer, heating element, molecular sieve layer, electrochemical salt bridge, or light-emitting layer. For example, a light emitting layer may comprise a diode and lens combination. Likewise, the invention is not limited to per se juxtaposition of elements in the electrical circuit. For example, interposed between disclosed elements of the circuit may be one or more resistors, inductors, capacitors, batteries, photovoltaic elements, transistors, diodes, transducers, amplifiers, thermocouplers, antennas, switches, heat sinks, transmission lines, or other electrical circuit components.

The term “in fluid communication” as used herein with reference to an element of the device refers to a state in which the device, after being wetted by a liquid sample, may permit the flow or diffusion of liquid sample or components of the liquid sample through or into one or more portions of that element. The term further refers to a condition in which two or more particular elements are wetted by the liquid sample. The term “in fluid communication” as applied herein to the conducting polymer component refers to a state in which the device, after being wetted by a liquid sample, may permit the diffusion of dopant onto or into the surface of the conducting polymer component. The term “in fluid communication” as applied herein to enhancement of doping of the conducting polymer component by means of an electrical potential applied across a doping facilitating electrode and its counterelectrode refers to a state in which the device, after being wetted by a liquid sample, may permit the diffusion of ions in response to the applied electrical potential.

The term “wetting” as used herein with reference to an element of the device refers to a state in which liquid sample has penetrated an element, such as fluid flow of a liquid sample through a binder support medium, except that with respect to a conducting polymer component the term “wetting” refers to a condition in which the surface of the conducting polymer component is in contact with liquid from a liquid sample. For example, a conducting polymer component may be in contact with and thus wetted by liquid from a liquid sample that is flowing through a binder support medium that is vertically juxtaposed above the conducting polymer component.

The term “doping facilitating electrode” as used herein refers to an element that is employed to apply an electrical potential in concert with at least one other electrode such that a dopant ion bearing a positive or negative charge may be attracted toward, onto the surface of, or into the mass of a conducting polymer component. In an illustrative embodiment, a doping facilitating electrode may be in electrical communication with a conducting polymer component, its counter electrode may be an electrode that is in electrical communication with a binder support medium but not in direct physical contact with the conducting polymer component, and neither of those electrodes would be an electrical lead of a readout circuit that comprises the conducting polymer component. In another illustrative embodiment, one or more of the electrical leads of a readout circuit may serve as a doping facilitating electrode, in concert with a counter electrode that is not an electrical lead of the readout circuit.

The terms “dopant” and “dopant compound” as used herein are interchangeable and refer to an ion or an uncharged chemical species that may adsorb onto or diffuse into a conducting polymer component, and whose association at the surface of or within a conducting polymer component may result in increased electrical conductivity in the conducting polymer component. The terms “dopant” and “dopant compound” are used herein without regard to the mechanism of conductivity enhancement. Thus, the dopant species may be more conductive than the pristine conducting polymer component and thereby passively increase the conducting polymer component's electrical conductivity upon doping, the dopant species may react with the conducting polymer component to generate mobilizable charges therein, or the dopant compound may provide some other mechanism for enhancement of the conducting polymer's electrical conductivity relative to its undoped state.

The term “unreacted dopant” refers to dopant that has not yet combined with a conducting polymer component with a resulting increase in electronic conductivity of the conducting polymer component, and the term “reacted dopant” refers to dopant that has combined in such a fashion.

The terms “dopant precursor” and “dopant precursor compound” as used herein are interchangeable and refer to a chemical compound or ion that can be converted into a dopant by means of a process that comprises one or more catalytic steps, and wherein the process is capable of being performed in a liquid sample in the device within the time period and under the conditions of the assay.

The terms “catalytic” and “catalyst” as used herein refers to a compound or composition of matter that is capable of lowering the activation energy of a chemical reaction but wherein no net change in the structure of the referenced compound or composition of matter is produced by the reaction. “Catalyst(s)” as used herein include but are not limited to enzymes that catalyze reduction-oxidation reactions. The term “reduction-oxidation” as used herein refers to catalysis in which the oxidation state of a product of interest is higher or lower than that of the chemical species that is converted to the product by the catalyst. The catalysis typically involves intermolecular transfer of electrons in conjunction with intermolecular transfer of hydrogen atoms or oxygen atoms. It should be understood that the yields of product produced in a catalyzed reaction depend upon the catalyst, substrate, product, equilibrium characteristics, and ambient conditions of the assay.

The term “conducting polymer component” refers to a polymeric material for which the electrical conductivity increases upon being doped by a dopant. Illustrative examples of conducting polymers include homopolymers, derivatives, analogs, copolymers, blends and composites of polymers such as polyacetylene (PA), polyphenylene (PPh), poly(phenylene vinylene) (PPV), poly(thienylene vinylene) (PThV), polypyrrole (PPy), polythiophene (PTh), polyaniline (PANI), poly(phenylene sulfide) (PPS), polyfuran (PF), polyisothianonaphthene (PIThNaph), polyazulene (PAz), polyacenes (PAc), polythiazine (SN), and melanins such as eumelanins and phaeomelanins. The conducting polymer component may be in a physical form of a film, bulk solid, open celled foam, closed-cell foam, fabric, fiber, wool, powder, mesh, composite, gel, slurry, interpenetrating network, laminate, as well as a coating on a substrate wherein the substrate consists essentially of a different substance.

The term “in electrical communication” as used herein refers to an element of the device that is capable of experiencing an applied electrical potential by means of electronic conduction through neighboring elements, or by means of ionic conduction through a liquid sample with which it is in fluid communication, or by a combination of these means.

The terms “readout device” and “readout mechanism” are used synonymously herein. The terms refer to a device or mechanism that is capable of registering an indication of the magnitude of a measured electrical property such as current, frequency, voltage differential, or optical intensity. For example, the indication may provide a simple positive or negative indication, e.g., an electronic display reading “PREGNANT” or “NOT PREGNANT”. In another example, the display may index the test results along a continuum such as a spectrum or concentration gradient. Illustrative readout displays include dials, electronic displays, liquid crystal displays (LCDs), light emitting diodes (LEDs), and other types of amperage or voltage metering devices.

Immunoassay Device

In one embodiment, a device is provided comprising:
a) a binder support medium comprising at least one detection zone for the detection of an analyte of interest, the binder support medium having a first side and a second side,
b) a conducting polymer component in fluid communication with the binder support medium, the conducting polymer component capable of having its electrical conductivity changed after being doped with a dopant molecule or compound;
c) a circuit that comprises a voltage source, such as a charged battery, wherein an electrical potential is maintained across at least one portion of one dimension of the conducting polymer component; and,
d) a readout mechanism that is capable of registering an indication of the magnitude of a measured electrical property in response to an electrical current

Typically, the dopant will increase the conductivity of the conducting polymer. In certain embodiments, the binder support medium may further comprise reagents that are capable of participating in or contributing to the generation of a dopant. In certain embodiments, the reagents are selected from a reduction oxidation enzyme, a first reduction-oxidation enzyme, a second reduction-oxidation enzyme, a first reducing agent, and a second reducing agent or a combination thereof, depending on the strategy utilized. In an alternative embodiment, the reagents are selected from the group consisting of a reduction-oxidation enzyme and a precursor molecule capable of conversion to a dopant upon interaction with the reduction-oxidation enzyme. In particular embodiments, at least one reagent capable of participating in or contributing to the generation of a dopant may be present within the detection zone. In one embodiment, the reagent present within the detection zone is a first reduction-oxidation enzyme. In another particular embodiment, the reduction-oxidation enzyme is a second reduction-oxidation enzyme. In a different embodiment, the binder support medium comprises a non-enzymatic reduction-oxidation catalyst that is capable of catalyzing the generation of a dopant molecule or of a dopant precursor molecule. In certain embodiments, the reagents present within the detection zone are selected from the group consisting of hydrogen peroxide, an iodide or iodine derived molecule such as potassium iodide, lactose peroxidase, glucose peroxidase, and glucose, or a combination thereof.

In certain embodiments, the binder support medium may further comprise additional zones. In one embodiment, the binder support medium may further comprise a tracer zone. In certain embodiments, the tracer zone may comprise a receptor moiety that is attached to a label (tracer) moiety and can bind to the analyte of interest, if present, in the liquid sample to form an analyte-labeled receptor complex. In an alternative embodiment, the tracer zone may comprise a competitor, wherein the competitor is an analyte of interest, an analogue, or derivative thereof bound to a tracer or label moiety, wherein the competitor contained in the tracer zone competes with the analyte of interest in the liquid sample, if present, in binding to the binder contained in the detection zone of the binder support medium. In certain embodiments, the label moiety can be a reagent such as discussed above that is capable of participating in or contributing to the generation of a dopant. In a particular embodiment, the label moiety is a reduction-oxidation enzyme. In a particular embodiment, the label is a first reduction-oxidation enzyme or a second reduction-oxidation enzyme. In a more particular embodiment, the label is selected from a lactose peroxidase enzyme and a glucose oxidase enzyme.

In one embodiment, the device may further comprise a tracer pad in fluid communication with the binder support medium, wherein the tracer pad is capable of accepting a liquid sample containing or lacking an analyte of interest. In certain embodiments, the tracer pad may comprise a receptor moiety attached to a label (tracer) moiety, wherein the receptor moiety can bind to the analyte of interest, if present, in the liquid sample to form an analyte-labeled receptor complex. In an alternative embodiment, the tracer pad may comprise a competitor, wherein the competitor is the analyte of interest, an analogue, or derivative thereof bound to a label moiety, wherein the competitor in the tracer pad competes with the analyte of interest in the liquid sample, if present, in binding to the binder contained in the detection zone of the binder support medium. In certain embodiments, the label moiety can be a reagent such as discussed above that is capable of participating in or contributing to the generation of a dopant. In a particular embodiment, the label moiety is a reduction-oxidation enzyme. In a particular embodiment, the label is a first reduction-oxidation enzyme or a second reduction oxidation enzyme. In a more particular embodiment, the label is selected from a lactose peroxidase enzyme and a glucose oxidase enzyme. In other embodiments, the tracer pad can further comprise additional reagents required in the generation of a dopant.

In one embodiment, the device may further comprise a reagent pad that is in vertical juxtaposition with the first side of or otherwise in fluid communication with the binder support medium. In certain embodiments, the reagent pad comprises reagents that are capable of participating in or contributing to the generation of a dopant. In particular embodiments, the reagent pad may comprise one or more reducing agents. In a particular embodiment, the reagent pad may compromise a first reducing agent and a second reducing agent. In a more particular embodiment, the reagent pad may comprise glucose and an iodine, iodide, or iodide derived molecule, such as potassium iodide. In an alternative embodiment, the reagent pad may comprise a dopant precursor molecule capable of conversion to a dopant upon interaction with a catalyst such as a reduction-oxidation enzyme. In a more particular embodiment, the dopant precursor molecule is hydrogen peroxide.

In one embodiment, the device may further comprise a sample pad that is capable of accepting a liquid sample. The sample pad may be in fluid communication with a tracer pad, if present, and with the binder support medium. In one embodiment, the device can further comprise a sump that is downstream from and in fluid communication with the binder support medium, and that is capable of absorbing excess liquid from the binder support medium. In additional embodiments, the device can comprise a housing that provides support for device.

The present invention can allow for a liquid sample containing or lacking an analyte of interest to be applied to the device, wherein the analyte interacts with reagents present in the device to produce a dopant capable of being detected. In a sandwich type assay, the analyte of interest, if present, can be bound by a labeled receptor forming an analyte-labeled receptor complex following the application of the sample to the device. The label of the labeled receptor can be a reagent capable of participating in or contributing to the generation of a dopant. In certain embodiments, the label can be a reduction-oxidation enzyme. In a particular embodiment, the label is a lactose peroxidase enzyme or a glucose oxidase enzyme. The analyte-labeled receptor complex can flow from the point of interaction to a detection zone on the binder support medium. The detection zone comprises an immobilized binder moiety. The binder can be a moiety capable of binding to the analyte-labeled receptor complex. In certain embodiments, the detection zone further comprises reagents capable of participating in or contributing to the generation of a dopant. In particular embodiments, the detection zone comprises a first reduction-oxidation enzyme or a second reduction-oxidation enzyme. As the liquid sample flows through the binder support medium, it also may encounter additional reagents capable of participating in or contributing to the generation of a dopant. In certain embodiments, additional reagents can include one or more reducing agents. For example, the additional reagents can be a first reducing agent and a second reducing agent, such as, for example, glucose and potassium iodide. Upon binding in the detection zone, the label of the analyte-labeled receptor complex participates in the generation of the dopant. In alternative embodiments, the assay can be formatted for use as a competitive assay, wherein the competitor is bound to a label that participates in the generation of a dopant.

In one embodiment, a reagent pad may be present in the device and be vertically juxtaposed to or otherwise in fluid communication with the binder support medium, and wherein the reagent pad comprises a first reduction agent and a second reduction agent, such as, for example, glucose and potassium iodide. Upon being wetted by the liquid sample, the reducing agents are capable of diffusing from the reagent pad into the detection zone of the binder support medium, where the reducing agents encounter the reduction-oxidation enzymes.

In an alternative embodiment, the reagent pad may comprise a precursor molecule capable of conversion to a dopant upon interaction with a reduction-oxidation enzyme, such as, for example, hydrogen peroxide. Upon being wetted by the liquid sample, the precursor molecule is capable of diffusing from the reagent pad into the detection zone of the binder support medium, where it encounters a reduction-oxidation enzyme.

In further embodiments, the following chemical compounds optionally may be contained in the binder support medium, tracer pad, reagent pad, or other storage medium that is in fluid communication with or comprised within the binder support medium through which liquid flow occurs: the receptor molecule that is bound to a first reduction-oxidation enzyme, the second reduction-oxidation enzyme, and the chemical compound that is transformed into a dopant precursor compound by the second reduction-oxidation enzyme. These chemical compounds optionally may be provided by transportation from the binder support medium, sample pad, tracer pad, reagent pad, or other storage medium by means of in-line liquid flow directly through the pad or other medium to the detector zone portion. These chemical compounds alternatively maybe provided by a storage pad or other medium through which in-line liquid flow does not occur, but which are nevertheless in fluid communication with the binder support medium (e.g., juxtaposed above it) such that the storage pad or other medium may be wetted by capillary action, followed by leaching of the chemical compounds from the storage pad or other medium into the area of in-line liquid flow. In any case, it is desirable that chemical compounds be provided in a manner that makes them timely available at the detection zone portion of the binder support medium.

Alternatively, one or more reagents may be provided in a controlled release format in the binder support medium or other element. For example a reagent may be provided in micropellets in soluble protective casings. The casing composition and its thickness may be selected so as to favor release of most of the reagent at a particular number of minutes after exposure to a liquid sample. This approach can prevent the premature reactions and minimize false positives in the device. For instance, in the first 3 minutes of an assay, labeled receptor from a tracer pad may be flushed downstream to points at and past the detection zone in minutes 5 to 10 of the assay, a dopant precursor may be released from micropellets that had been encased in a soluble composition of matter in the detection zone or in a reagent pad juxtaposed to the detection zone.

The present invention relies on a chemical reaction or series of chemical reactions that occur in the detection zone to generate a dopant. In one embodiment, a first reduction-oxidation enzyme is capable of oxidizing the first reducing agent in the presence of oxygen, generating an intermediate compound. The second reducing agent is then oxidized by the second reduction-oxidation enzyme in the presence of the intermediate, creating the dopant. In certain embodiments, the series of chemical reactions occurs within the detection zone of the binder support medium upon the binding of the analyte-labeled receptor complex, or an analyte, homologue or derivative thereof bound to a label, wherein the label of the labeled receptor complex, or the label bound to the analyte, homologue, or derivative thereof, is a first reduction-oxidation enzyme.

Alternatively, a single chemical reaction may generate a dopant. In one embodiment, a precursor molecule is capable of conversion to a dopant upon interaction with a reduction-oxidation enzyme. The precursor molecule is oxidized by the reduction-oxidation enzyme to create the dopant. In a particular embodiment, the precursor molecule is hydrogen peroxide and the reduction-oxidation enzyme is lactose peroxide.

The dopant is capable of diffusing within the binder support medium and into or onto a conducting polymer component that is in fluid communication with the binder support medium. The conducting polymer component is connected to a circuit and serves as the gate or switch of the circuit. Upon reaction with the dopant, the conducting polymer component, held at an applied electrical potential, has an electrical conductivity that scales with the degree of doping. The conductivity increase may be quantitatively measured, e.g., by a digital mechanism, or a conductivity change, e.g., an increase, is indicated as being at a threshold level, e.g., by emission of light from a light source such as a bulb. In certain embodiments, an electronic readout mechanism will display a result in words or symbols, such as “pregnant”/“not pregnant”, or a number indicating concentration or amount. The operator, after adding the liquid sample to the device, waits for an indication from the readout mechanism to determine the presence or amount of the analyte of interest.

In one embodiment, the conducting polymer component is in electrical contact with at least three electrical leads. Two of the electrical leads may comprise the positive and negative electrical leads for an electrical circuit that comprises a readout mechanism for monitoring conductivity changes in the conducting polymer component. A third electrical lead may be employed as a doping-facilitating electrode in combination with a counterelectrode that is in electrical contact with the liquid sample as the liquid sample flows through the device. Upon applying an electrical potential across the third electrical lead and its counterelectrode, doping of the conducting polymer component by a dopant molecule may be expedited. Dopant anions such as triiodide may be attracted toward a positively polarized doping facilitating electrode, which may be placed internally to the conducting polymer component or for instance may be juxtaposed below a conducting polymer component that is juxtaposed on its upper side to a binder support medium. Alternatively, dopant cations may be attracted toward a negatively polarized doping facilitating electrode.

In one embodiment, the present invention comprises the application of a liquid sample containing or lacking an analyte of interest to a sandwich-type immunoassay device, wherein the analyte, if present, would be capable of interacting with a labeled receptor molecule to form an analyte-labeled receptor complex, and wherein the label of the labeled receptor is a reduction-oxidation enzyme. In a particular embodiment, the reduction-oxidation enzyme can be a first reduction-oxidation enzyme. The analyte-receptor complex then flows to a detection zone on a binder support medium, where it may be immobilized by means of a ligand-receptor interaction between an immobilized binder and the analyte-receptor complex. Upon binding of the analyte-receptor complex in the detection zone, at least one chemical compound may be capable of undergoing a transformation by means of the first oxidation-reduction enzyme to form a precursor to a dopant compound. The precursor may be capable of being transformed into a dopant compound by means of a second reduction-oxidation enzyme or other redox catalyst that may be present in the detection zone. Alternatively, a precursor molecule capable of being converted into a dopant may be present in the detection zone. The reduction-oxidation enzyme bound to the receptor molecule of the analyte-receptor complex interacts with the precursor molecule to generate the dopant. Once generated, the dopant may be capable of converting a conducting polymer component, which is present in communication with the detection zone of the binder support medium, from a non-conducting state to a conducting state. The conversion of the conducting polymer component to a conducting state by the dopant may increase the conducting polymer component's electronic conductivity, which can be indicated by a readout mechanism.

In one embodiment, the present invention comprises the application of a liquid sample containing or lacking an analyte of interest to a competitive-type immunoassay device, wherein the analyte, if present, competes with a competitor, wherein the competitor is an analyte, analyte analogue, or derivative thereof, contained in the device for the immobilized binder in the detection zone. The competitor contained in the device may be bound to a label, directly or indirectly through a receptor. In one embodiment, the label can be a reduction-oxidation enzyme. The amount of dopant generated in the competitive type assay is generally inversely proportional to the amount of analyte present in the liquid sample.

Methods for Detecting an Analyte of Interest

In one embodiment, the present invention comprises a method for detecting the presence or amount of an analyte of interest in a two-enzyme sandwich assay comprising:

a) applying a liquid sample containing or lacking an analyte of interest to an immunoassay device;

b) reacting the analyte of interest, if present, with a labeled receptor molecule (tracer), the label being a first reduction-oxidation enzyme, wherein the receptor molecule and label are supported within the assay device in a movable manner, to form an analyte-labeled receptor complex;

c) transporting the analyte-labeled receptor complex through a binder support medium by means of liquid flow to a detection zone portion of the binder support medium;

d) immobilizing the analyte-labeled receptor complex in the detection zone portion of the binder support medium whereby the analyte portion of the analyte-labeled receptor complex binds to a binder by means of a ligand-receptor interaction;

e) removing by liquid flow from the detection zone portion first reduction-oxidation enzyme that is not present in an analyte-labeled receptor complex immobilized in the detection zone portion by a binder, wherein the removal by liquid flow may occur prior to or concurrent with any of the events below;

f) transforming at least one chemical compound by the first reduction-oxidation enzyme portion of the immobilized analyte-labeled receptor complex, to form a dopant precursor molecule;

g) transforming the dopant precursor molecule to a dopant molecule by reaction with a second reduction-oxidation enzyme that is present within the detection zone portion h) diffusing the dopant into or onto the surface of a conducting polymer component that is in fluid communication with the detection zone portion, to form a doped conducting polymer component;

i) detecting the degree of doping of a conducting polymer component by an electrical potential applied across the conducting polymer component, whereby a change in the electrical current or electrical conductivity of the conducting polymer component for a given amount of applied electrical potential indicates the degree of doping.

In an alternative embodiment, the present invention comprises a method for detecting the presence or amount of an analyte of interest utilizing a one-enzyme sandwich assay comprising:

a) applying a liquid sample containing or lacking an analyte of interest to an immunoassay device;

b) reacting the analyte of interest, if present, with a labeled receptor molecule (tracer), the label being a reduction-oxidation enzyme, wherein the receptor molecule and label are supported within the assay device in a movable manner, to form an analyte-labeled receptor complex;

c) transporting the analyte-labeled receptor complex through a binder support medium by means of liquid flow to a detection zone portion of the binder support medium;

d) immobilizing the analyte-labeled receptor complex in the detection zone portion of the binder support medium whereby the analyte portion of the analyte-labeled receptor complex binds to a binder by means of a ligand-receptor interaction;

e) removing by liquid flow from the detection zone portion reduction-oxidation enzyme that is not present in the analyte-labeled receptor complex immobilized in the detection zone, wherein the removal by liquid flow may occur prior to or concurrent with any of the events below;

f) transforming at least one chemical precursor dopant molecule by the reduction-oxidation enzyme portion of the immobilized analyte-labeled receptor complex to form a dopant;

g) diffusing the dopant into or onto the surface of a conducting polymer component that is in fluid communication with the detection zone portion, to form a doped conducting polymer component;

h) detecting the degree of doping of a conducting polymer component by an electrical potential applied across the conducting polymer component, whereby a change in the electrical current or electrical conductivity of the conducting polymer component for a given amount of applied electrical potential indicates the degree of doping.

In still another embodiment, the present invention provides methods for detecting the presence or amount of an analyte of interest in a two-enzyme competitive type assay comprising:

a) applying a liquid sample containing or lacking an analyte of interest to an immunoassay device, wherein the immunoassay device contains a competitor bound to a label forming a competitor complex, the competitor being an analyte, analogue, or derivative thereof, and the label being a first reduction-oxidation enzyme;

b) transporting the competitor complex and analyte contained in the liquid sample, if present, through a binder support medium by means of liquid flow to a detection zone portion of the binder support medium, whereby the competitor portion of the competitor complex and the analyte contained in the liquid sample compete for the binding of the binder in the detection zone by means of a ligand-receptor interaction;

c) removing by liquid flow from the detection zone portion competitor complex that is not immobilized in the detection zone, wherein the removal by liquid flow may occur prior to or concurrent with any of the events below;

d) transforming at least one chemical compound by the first reduction-oxidation enzyme portion of the immobilized competitor complex, to form a dopant precursor molecule;

e) transforming the dopant precursor molecule to a dopant by reaction with a second reduction-oxidation enzyme that is present within the detection zone portion;

f) diffusing the dopant into or onto the surface of a conducting polymer component that is in fluid communication with the detection zone portion, to form a doped conducting polymer component;

g) detecting the degree of doping of a conducting polymer component by means of an electrical potential applied across the conducting polymer component, whereby a change in the electrical current or electrical conductivity of the conducting polymer component for a given amount of applied electrical potential indicates the degree of doping.

In yet another embodiment, the present invention provides methods for detecting the presence or amount of an analyte of interest in a one-enzyme competitive type assay comprising:

a) applying a liquid sample containing or lacking an analyte of interest to an immunoassay device, wherein the immunoassay device contains a competitor bound to a label forming a competitor complex, the competitor being an analyte, analogue, or derivative thereof, and the label being a reduction-oxidation enzyme;

b) transporting the competitor complex and analyte contained in the liquid sample, if present, through a binder support medium by means of liquid flow to a detection zone portion of the binder support medium, whereby the competitor portion of the competitor complex and the analyte contained in the liquid sample compete for the binding of the binder by means of a ligand-receptor interaction;

c) removing by liquid flow from the detection zone portion competitor complex that is not immobilized in the detection zone portion, wherein the removal by liquid flow may occur prior to or concurrent with any of the events below;

d) transforming of at least one chemical precursor dopant molecule by the reduction-oxidation enzyme portion of the immobilized competitor complex, to form a dopant;

e) diffusing of a dopant molecule into or onto the surface of a conducting polymer component that is in fluid communication with the detection zone portion, to form a doped conducting polymer component;

f) detecting of the degree of doping of a conducting polymer component by means of an electrical potential applied across the conducting polymer component, whereby a change in the electrical current or electrical conductivity of the conducting polymer component for a given amount of applied electrical potential indicates the degree of doping.

Figures

Referring now to the drawings, in which like numerals represent like elements throughout the several Figures, aspects of the invention and the illustrative operating environment will be described. The Figures, while representative of certain embodiments of the invention, are not intended to limit the invention in any way.

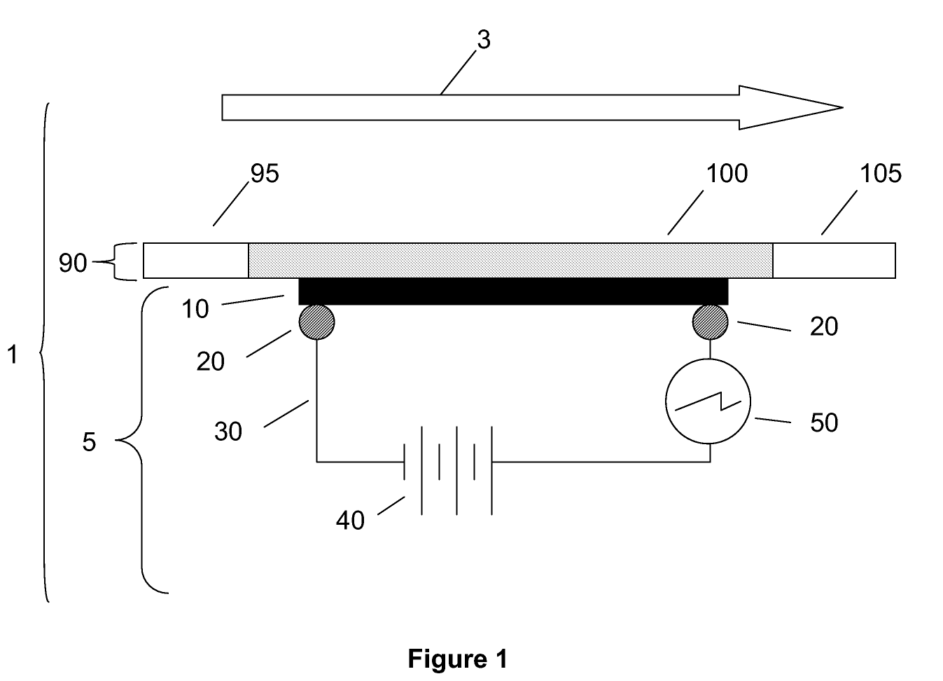

FIG. 1 is a schematic illustrating one embodiment of an immunoassay device 1 comprising an electrical configuration 5 comprising a conducting polymer component 10, and a binder support medium 90 that can be utilized for detecting an analyte of interest in a liquid sample.

The electrical configuration 5 comprises a conducting polymer component 10, wherein the conducting polymer component 10 is in fluid communication with a binder support medium 90. In a particular embodiment, the conducting polymer component 10 may be polyaniline. In particular embodiments, the physical forms of conducting polymer component 10 comprise high surface areas and are characterized by relatively rapid absorption of dopants.

The conducting polymer component 10 is maintained in electrical contact with at least two electrical leads 20 sufficient to apply an electrical potential. Electrical leads 20 suitable for this purpose are familiar to those in the art; and for example include leads provided by contact of the conducting polymer component 10 with solder, banana clips, clips such as metal or graphite clips, conductive pastes such as a graphite paste, conductive adhesives such as an adhesive containing black carbon filler or powdered silver or aluminum, pure metal or alloy in a soft or liquid form such as mercury, and other means for providing electrical contact between two conducting materials.

In electrical configuration 5 at least two electrical leads 20 that are in electrical contact with conducting polymer component 10 are also in electrical contact with a circuit 30 such that the conducting polymer component 10 serves as a gate for current. If a dopant is generated, the conducting polymer component 10 may become doped and conductive, allowing a current to flow through the circuit 30. In the absence of a dopant, the conducting polymer component 10 may be undoped and non-conductive. Because the conducting polymer component 10 may be non-conductive in an undoped form, a current may be unable to flow through the circuit 30. (Alternatively, the polymer component 10 may be partly conductive, wherein doping will increase the conductivity and the amount of current). Types of circuits 30 that may be utilized may be configured in any manner familiar to persons of skill in the art. For example, the circuit 30 may be configured as assembled parts such as a type used on a bread board, a monolithic circuit as used a printed circuit board, or an integrated circuit as used on a chip.

During operation of the immunoassay device 1 a potential may be maintained across the conducting polymer component 10 by means of electrical leads 20 and a voltage source 40 in the circuit. Examples of a suitable voltage source 40 include a battery, capacitor, supercapacitor, electrochemical capacitor, fuel cell, photovoltaic source, direct current generator, alternating current generator, and other voltage sources such as are common in the arts of energy storage and energy management.

During operation of the immunoassay device 1 the flow of current through the circuit 30 can be monitored by means of a readout mechanism 50 in the circuit 30. In one embodiment, the readout mechanism 50 may be a switch that is toggled on and or off at particular current thresholds. In another embodiment, the readout mechanism 50 may comprise a variable resistor to determine the level of current across a discrete continuum of amperage. The location of the readout mechanism 50 and the voltage source 40 relative to each other may be varied; for instance, the readout mechanism 50 may either precede or follow the voltage 40 in series in the circuit 30 as current flow is measured by the flow of electrons. Alternatively, the readout mechanism 50 may be in a separate circuit that is electrically in parallel to circuit 30. For example, readout mechanism 50 might be in electrical communication with circuit 30 by means of a parallel inductor circuit or by means of an electromagnet in parallel.

The immunoassay device 1 of FIG. 1 further comprises a binder support medium 90. The binder support medium 90 can be in fluid communication with the conducting polymer component 10. The binder support medium 90 may be comprised of three or more portions: a first portion 95 that may be capable of receiving a liquid sample containing or lacking an analyte of interest; a detection zone 100 comprising an immobilized binder, and wherein dopant molecules may be generated; and a third portion 105 capable of receiving liquid flow.

The particular dimensions of the binder support medium 90 can be a matter of convenience, depending upon the size of the liquid sample involved, the assay protocol, the choice of conducting polymer component 10, and the like. The binder support medium 90 used with the invention can be in the form of strips, columns, circles, sheets, ovals, squares or other forms suitable for the particular assay. In a particular example the binder support medium 90 may be a horizontal nitrocellulose strip supporting lateral flow. The first portion 95, detection zone 100, and the third portion 105 of the binder support medium 90 can be on the same dimensional material or on separate dimensional materials. For example, the two portions can be on the same strip of binder support medium, or the first portion 95 can be on one binder support medium strip, and the third portion 105 can be on a separate binder support medium strip, wherein the two strips are laterally juxtaposed and the detection zone 100 abuts the third portion 105 of the binder support membrane.

The detection zone 100 may encompass an area less than the whole of the first portion 95 of the binder support medium 90, or may encompass the entire first portion 95 of the binder support medium 90. In certain embodiments, the first portion 95 of the binder support medium 90 may comprise more than one detection zone 100, wherein each detection zone can be capable of detecting a different analyte of interest. The binder support medium 90 may be juxtaposed to or otherwise in fluid communication with a conducting polymer component 10; in this embodiment they are vertically juxtaposed and in the depicted embodiment the detection zone 100 is the only portion of the binder support medium 90 that is juxtaposed to a conducting polymer component 10, however the invention is not so limited. The polymer component 10 may be larger, smaller or the same in area as the detection zone 90, and one of the two may overlap the other.

The first portion 95 and the detection zone 100 may also serve more than one role, for instance they may serve as wicking elements, or as reservoirs of dry reagents and catalysts in anticipation of dissolution by a liquid sample. Any portion of the binder support medium 90 may also serve as a structural element for the immunoassay device 1. For instance in addition to the conducting polymer component 10, some or all parts of the electrical configuration 5 may be juxtaposed to, affixed to, or otherwise support or be supported by one or more of the first portion 95, detection zone 100, and third portion 105.

The detection zone 100 can be juxtaposed to the conducting polymer component 10. Non limiting examples of the juxtapositions contemplated by the invention include vertical juxtaposition of a detection zone 100 pad above a conducting polymer component 10 pad; lateral juxtaposition between alternating stripes of detection zone 100 and conducting polymer component 10; packed particles of conducting polymer component 10 characterized by the presence of detection zone 100 medium in the interstitial spaces; dispersal of detection zone 100 through an open cell foam of conducting polymer component 10; an interpenetrating network of conducting polymer component 10 and detection zone 100; wire-shaped or ribbon-shaped conducting polymer components 10 penetrating through detection zone 100; and other configurations. The detection zone 100 may have one or more additional layers interposed in juxtaposition between the detection zone 100 and the conducting polymer component 10, such as an interposed adhesive layer that permits penetration by dopant or such as an interposed sacrificial layer that protects the surface of the conducting polymer component 10 when dry but which dissolves in the presence of a liquid sample. Alternatively the assay configuration may comprise a space or channel interposed between detection zone 100 and conducting polymer component 10 such that detection zone 100 and conducting polymer component 10 are not in physical contact, or such that their interface is not in complete physical contact, and whereby a gas such as air, a fluid such as a liquid sample, a solid such as pH paper or enzyme-labeled microparticles, or another substance could pass unimpeded through or optionally fill the space or channel defined between detection zone 100 and conducting polymer component 10.

In brief, the utilization of a device as represented by FIG. 1 comprises supplying a liquid sample to the first portion 95 of the binder support medium 90; the liquid in the liquid sample moves in the direction of fluid flow 3 into a detection zone 100, and from there into the third portion 105 of the binder support medium 90. The direction of fluid flow 3 is determined by capillary forces, wicking, gravity, applied or ambient pressure or other driving force; it will be seen that the direction of fluid flow 3 proceeds from the first portion 95 of the binder support medium 90, through the detection zone 100, and continuing into or through the third portion 105 of the binder support medium 90. This fluid flow 3 may provide a self-flushing function during the immunoassay, thus the third portion 105 of the binder support medium 90 may receive unreacted or incompletely reacted reagents so that false positive signals are minimized in detection zone 100. Thus, this third portion 105 also may function in full or in part as a sump.

The generation of a dopant in the detection zone 100 may then trigger a response in the conducting polymer component 10, permitting electrical current (or increased electrical current) to flow through the electric configuration 5. The electrical current flows through the conducting polymer component 10, the electrical leads 20, the circuit 30, and the readout mechanism 50, not necessarily in that order, wherein the current flow is driven by the voltage source 40. The flow of electrical current through the electrical configuration 5 results in a measurable change in conduction through the circuit as indicated by the readout mechanism 50.

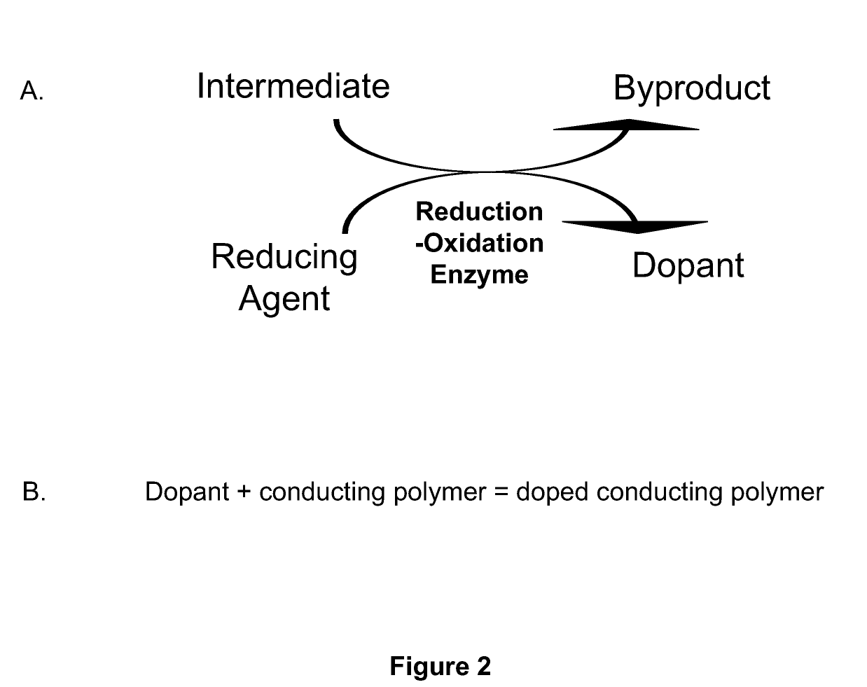

FIG. 2 is a schematic illustrating one embodiment of an immunoassay for a one-enzyme system; it may be understood to illustrate by an equation an example of the process of chemical conversion and doping. In Step A of FIG. 2 the chemical equation depicts a reducing agent (i.e., an electron donor) that is being converted to a dopant; here the catalysis involves an oxidation (i.e., having electrons extracted from the electron-donating reducing agent) to form a dopant. The top half of the same equation depicts a counterbalancing reaction; it should be understood that electrons extracted in the bottom portion of the equation may be injected into the chemical intermediate of the top half of the equation to yield a byproduct; a catalyst, depicted here as a reduction-oxidation enzyme, may facilitate the coupling of the two half reactions to obtain an efficient exchange of protons.

Step B of FIG. 2 summarizes the doping step. The dopant molecule may undergo a diffusion step that transports it onto the surface of or into the mass of a conducting polymer; the dopant after penetration of the conducting polymer may be identical to or different from the chemical structure of the dopant prior to that penetration. The resulting doping of the conducting polymer may then be indicated by a measurable flow of electrical current through a circuit as indicated by a readout mechanism. The formation and diffusion of dopant may thus serve as a detectable proxy for the presence of the reducing agent and the catalyst. Thus where the catalyst exists in a relationship with a receptor or competitor for the analyte, the catalytic activity may be the underlying diagnostic feature.

The dopant may be for instance an acid such as a Bronsted acid or Lewis acid, a metabolic carboxylic acid, triiodide, or other dopant. The reducing agent may be any molecule that is capable of conversion to a dopant by catalytic means under the operating conditions of the assay. Thus for instance the reducing agent for triiodide may be iodide anion.

The catalyst may be in a relationship with a receptor or competitor. The catalyst may be soluble or insoluble, mobile or immobilized, conjugated to a receptor or unconjugated, and may be modified or unmodified.

In one embodiment, the catalyst is a reduction-oxidation enzyme in an association with a receptor or competitor. The reduction-oxidation enzyme may further be associated with cofactors. The reduction-oxidation enzyme may be an artificial enzyme, an enzyme comprised of nucleic acids such as ribonucleic acids and deoxyribonucleic acids, or may be an amino acid-based enzyme, and may be a catalytic antibody. In particular the reduction-oxidation enzyme may be an enzyme such as a lyase, ligase, transferase, hydrolase, isomerase, and reduction-oxidation enzyme.

Although the catalyst in FIG. 2 is depicted as a reduction-oxidation enzyme, the invention is not so limited. Reduction-oxidation catalysts that are not enzymes are also contemplated by the invention, including but not limited to inorganic reduction-oxidation catalyst, organometallic reduction-oxidation catalyst, or organic reduction-oxidation catalysts that are not enzymes.

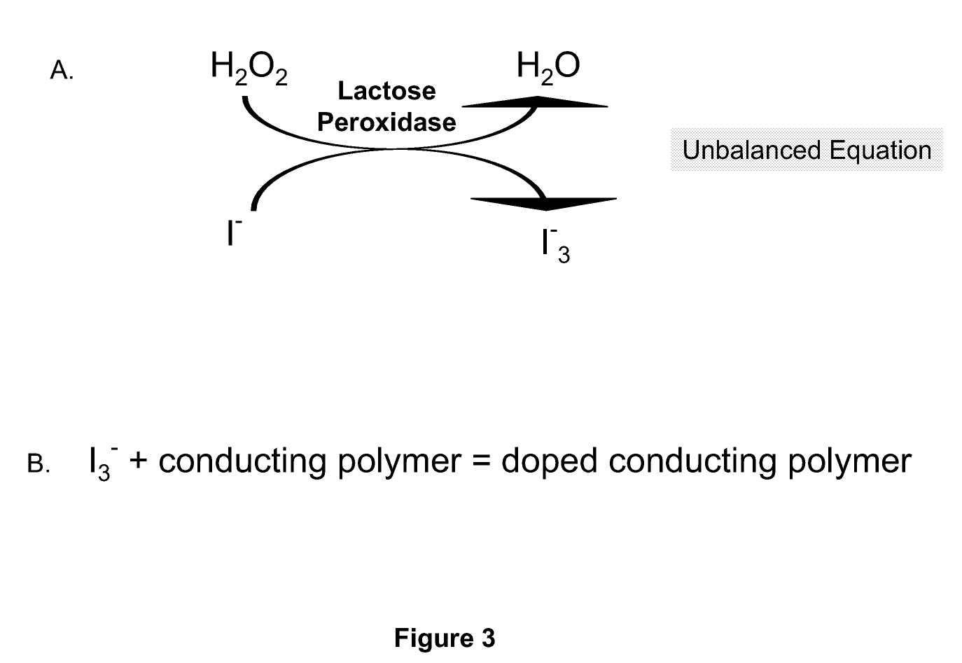

FIG. 3 is a schematic illustrating a particular embodiment of the immunoassay in FIG. 2. In Step A the dopant is triiodide anion (I₃⁻) and the reducing agent from which the dopant is generated is iodide anion (I⁻). The counterbalancing reaction is the conversion of hydrogen peroxide to water, and the reduction-oxidation catalyst is the reduction-oxidation enzyme lactose peroxidase. For purposes of simple illustration the equation as depicted is unbalanced. The triiodide anion as dopant may penetrate a conducting polymer to dope it, as illustrated in Step B.

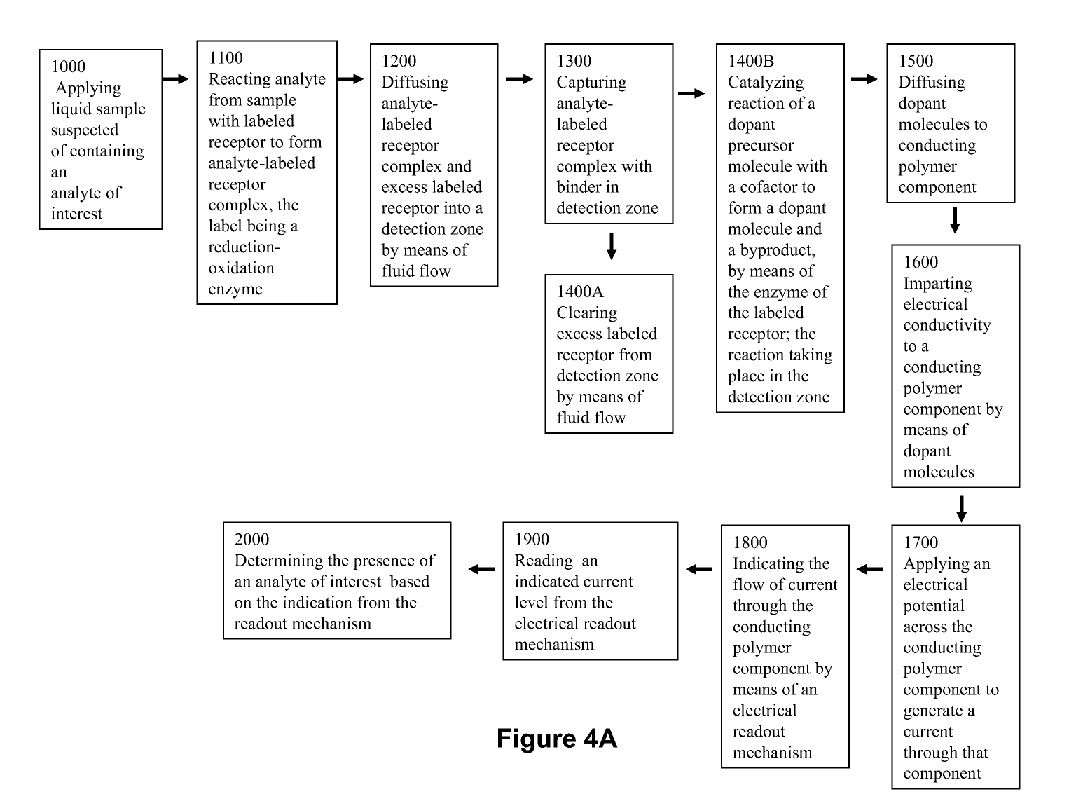

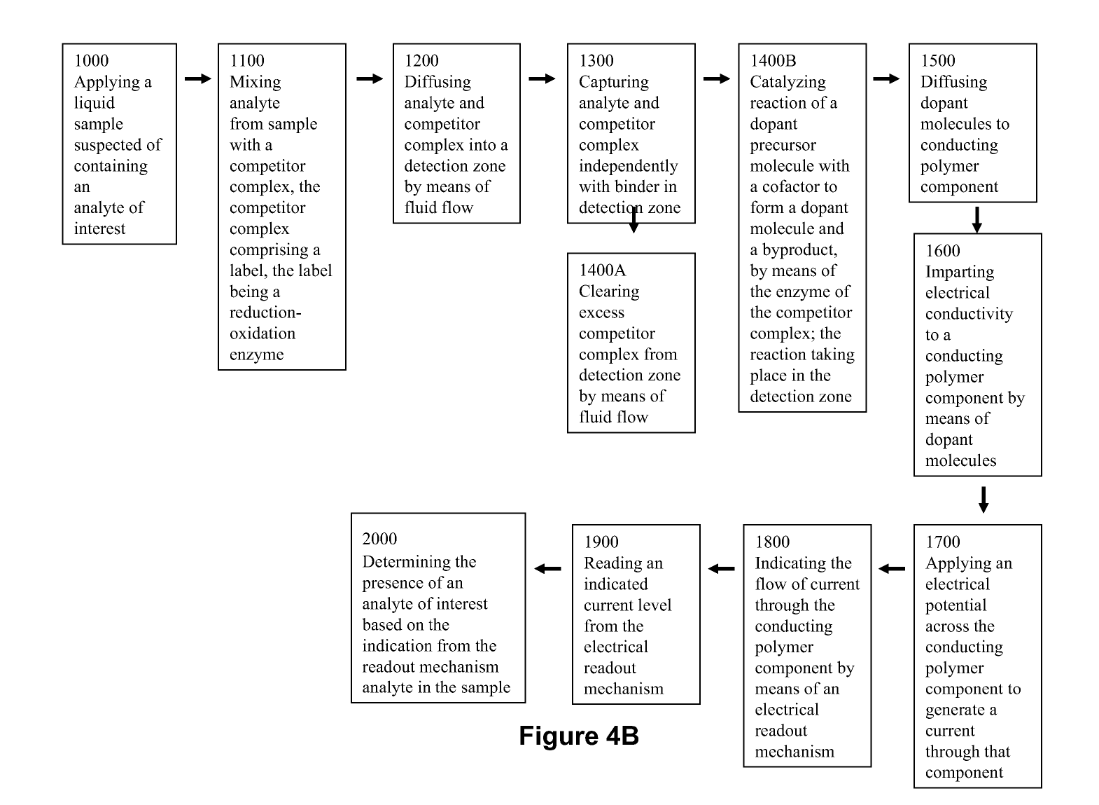

FIG. 4A is a flow chart illustrating one embodiment of a sandwich immunoassay for a one-enzyme system; it may be understood to illustrate chronologically an example of the assay sequence. In step 1000, apply a liquid sample suspected of containing an analyte of interest to the assay. In step 1100, react the analyte, if present in the liquid sample, with a labeled receptor to form an analyte-labeled receptor complex. The label of the labeled receptor is a reduction-oxidation enzyme. The labeled receptor may be present in dry form in the assay, and capable of dissolving in the liquid sample at the time of application. In one embodiment the enzyme label may be a lactose peroxidase enzyme. In step 1200, diffuse the analyte-labeled receptor complex into a detection zone by means of fluid flow. Because the sample is a liquid, the sample's liquid may be exploited to provide the fluid flow of the immunoassay. In step 1300, capture the analyte-labeled receptor complex with immobilized binder molecules which immobilize the complex in the detection zone. In step 1400A, clear any unbound labeled receptor from the detection zone by means of fluid flow. In this and other embodiments, (this may be done as part of the liquid sample flow, in which case no special or additional steps are needed. Alternatively, additional fluid can be provided to promote such clearing.) The excess labeled receptor is washed downstream from the detection zone. In step 1400B, catalyze a dopant precursor molecule with a cofactor utilizing the now immobilized label of the labeled receptor, which is a reduction-oxidation enzyme. This catalysis reaction forms a dopant molecule.

In step 1500, diffuse the dopant molecule produced in the catalysis reaction to a conducting polymer component. In this and other embodiments, the device structure is generally designed to allow such diffusion to occur without the need for specific actions. In step 1600, impart electrical conductivity to a conducting polymer component due to doping of the polymer with the diffused dopant molecule produced in the catalysis reaction. The doping of the polymer allows an electrical potential to pass through the conducting polymer. In step 1700, apply an electrical potential applied across the conducting polymer component to generate an electrical current through the conducting polymer component. The applied potential may be one that is held constant prior to and throughout the assay of the liquid sample, or may be applied at one or more convenient times during the assay. In step 1800, indicate the flow of current through the conducting polymer by means of an electrical readout mechanism. The amount of electrical current that is transmitted by the conducting polymer component can be detected and indicated by the electrical readout mechanism. In step 1900, read the indication level from the electrical readout mechanism. In step 2000, determine the presence of an analyte based on the indication from the readout mechanism.

FIG. 4B is a flow chart illustrating one embodiment of a competitive immunoassay for a one-enzyme system; it may be understood to illustrate chronologically an example of the assay sequence. In step 1000, apply a liquid sample suspected of containing an analyte of interest to the assay. In step 1100, mix the analyte from the sample with a competitor complex contained in the assay device. The competitor complex is a competitor comprising an analyte, analogue, or derivative thereof that is incapable of binding to the analyte of interest, but is capable of competing with the analyte of interest from the sample in binding an immobilized binder in the detection zone. The competitor complex further comprises a label, the label being a reduction-oxidation enzyme. In a particular embodiment, the reduction-oxidation enzyme is lactose peroxidase enzyme. The competitor complex may be present in dry form in the assay, and capable of dissolving in the liquid sample at the time of application. In step 1200, diffuse the competitor complex into a detection zone by means of fluid flow. Because the sample is a liquid, the sample's liquid may be exploited to provide the fluid flow of the immunoassay. In step 1300, capture the analyte and the competitor complex with immobilized binder molecules which immobilize the analyte and complex in the detection zone. The competitor complex and analyte are both capable of binding to the immobilized binder in the detection zone. Thus, the analyte and the competitor complex compete for the binder. In step 1400A, clear any competitor complex that is not bound to an immobilized binder from the detection zone by means of fluid flow. The excess competitor complex is washed downstream from the detection zone. In step 1400B, catalyze a dopant precursor molecule with a cofactor utilizing the now immobilized label of the competitor complex, which is a reduction-oxidation enzyme. The reaction occurs in the detection zone. This catalysis reaction forms a dopant molecule.

In step 1500, diffuse the dopant molecule produced in the catalysis reaction to a conducting polymer component. In step 1600, impart electrical conductivity to a conducting polymer component by doping the polymer with the diffused dopant molecule produced in the catalysis reaction. The doping of the polymer allows an electrical potential to pass through the conducting polymer. In step 1700, apply an electrical potential applied across the conducting polymer component to generate an electrical current through the conducting polymer component. The applied potential may be one that is held constant prior to and throughout the assay of the liquid sample, or may be applied at one or more convenient times during the assay. In step 1800, indicate the flow of current through the conducting polymer by means of an electrical readout mechanism. The amount of electrical current that is transmitted by the conducting polymer component can be detected and indicated by the electrical readout mechanism. In step 1900, read the indication level from the electrical readout mechanism. In step 2000, determine the presence of an analyte based on the indication from the readout mechanism.

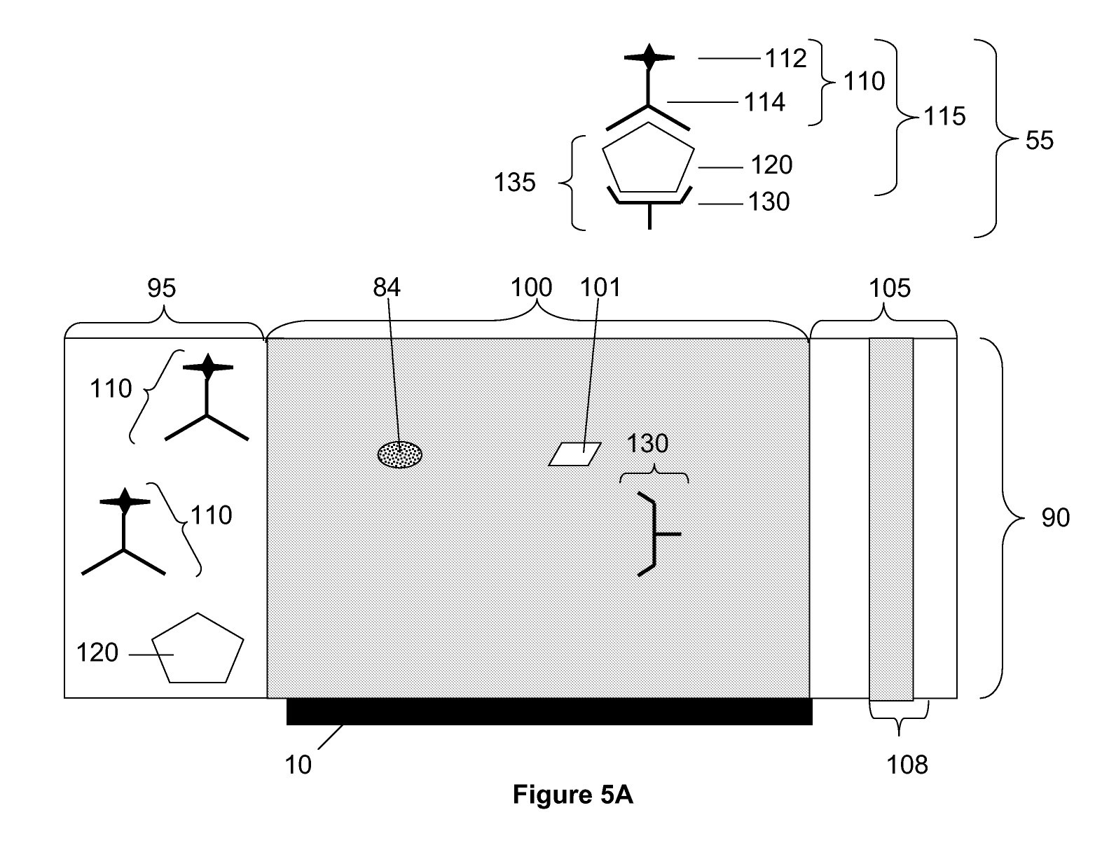

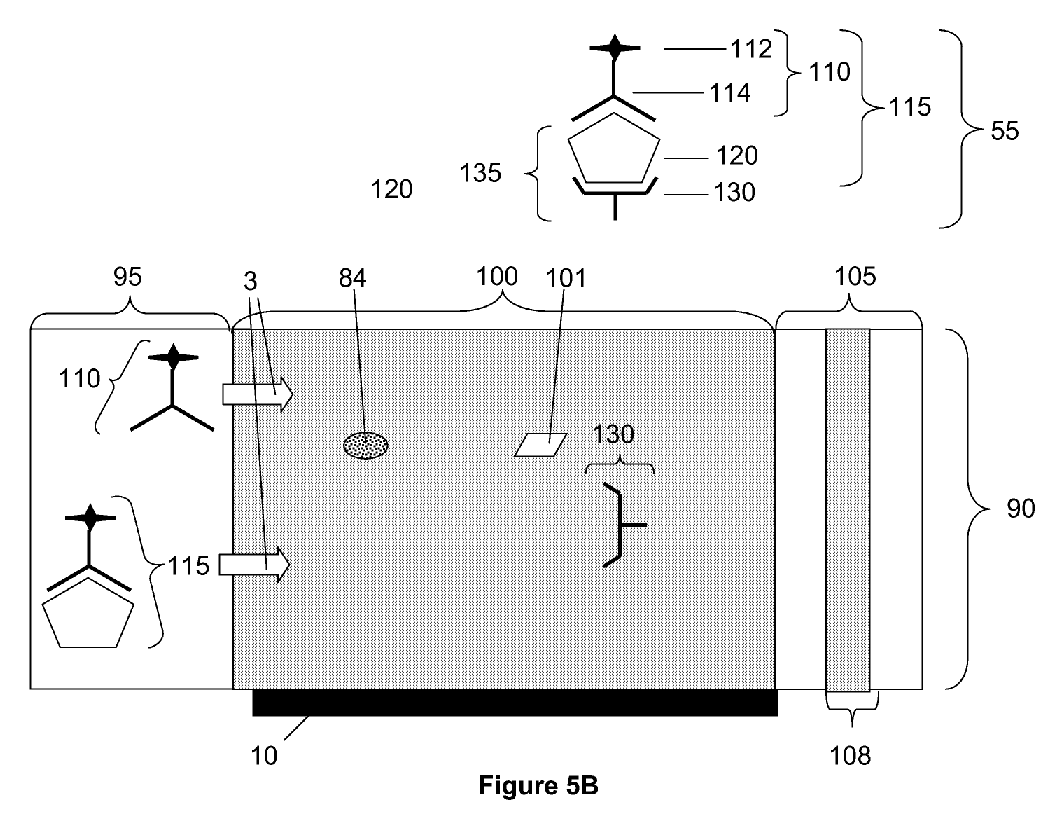

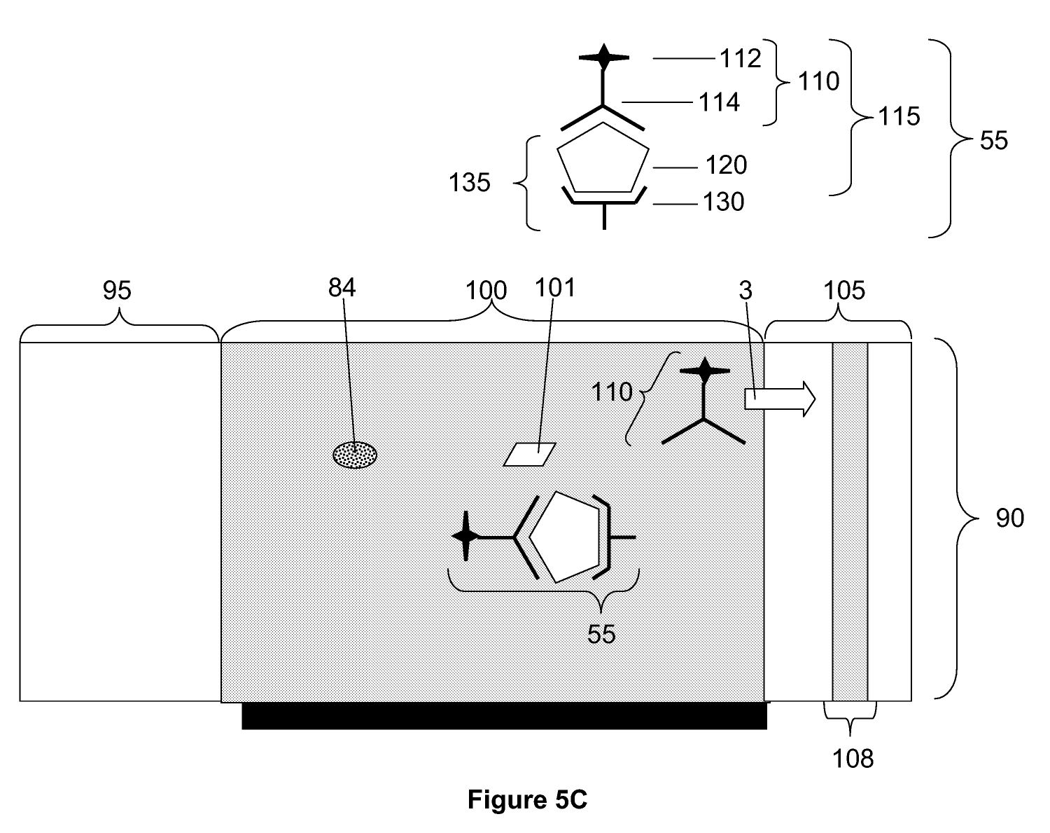

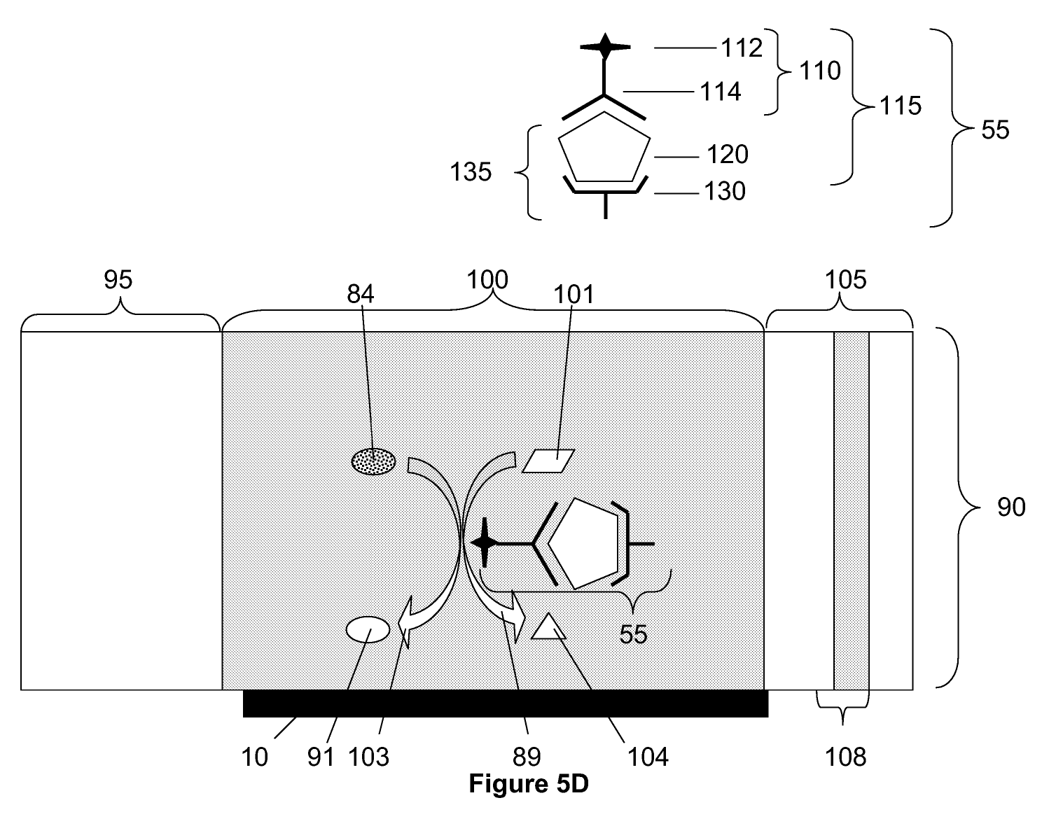

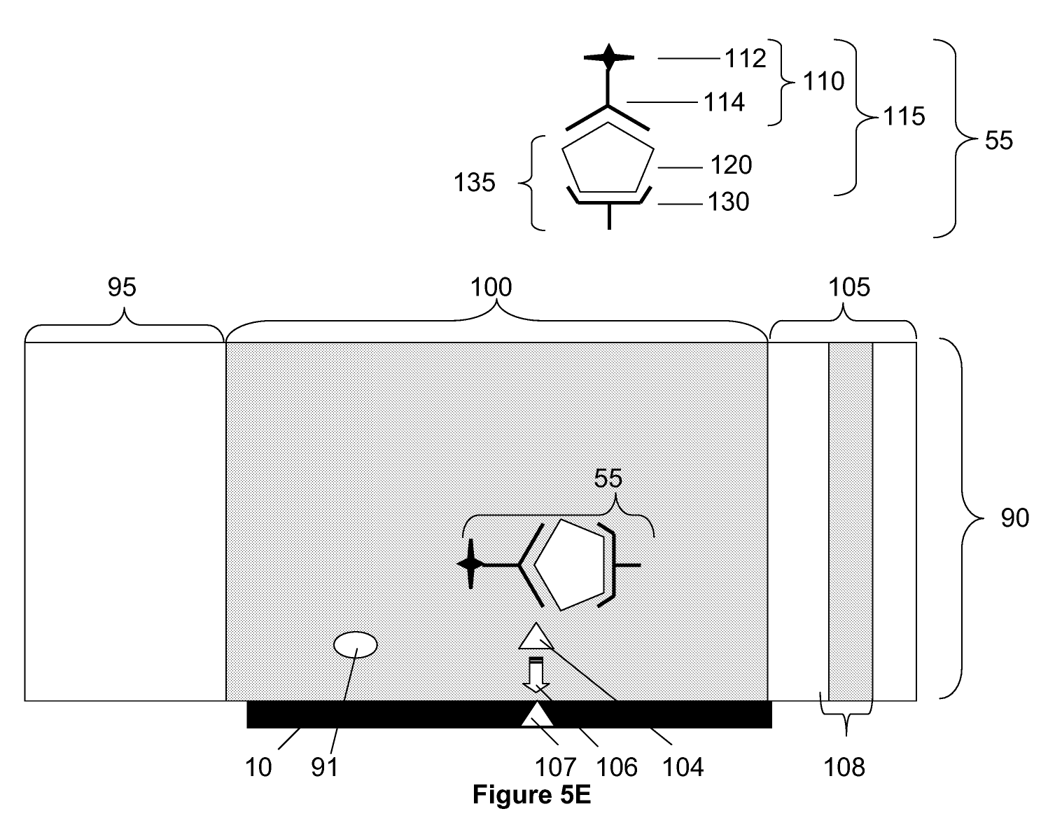

FIGS. 5A-5E are schematics illustrating one embodiment of an immunoassay, comparable to FIGS. 1 and 2, but illustrating some general features for a one-enzyme sandwich assay that may generate a chemical detection event. FIG. 5A depicts the initial state after application of a liquid sample into a first portion 95 of a binder support medium 90, but before significant binding by a labeled receptor 110 to an analyte 120, and before significant fluid flow into the detection zone 100 has occurred. A detection zone 100 may contain binder 130 specific to the analyte 120, and may contain a first chemical product 84 and dopant precursor compound 101. A third portion 105 of the binder support medium 90 may contain an optional control zone 108. As shown in FIGS. 5B and 5C, a liquid sample moving in the direction of fluid flow 3 enters the detection zone 100, and third portion 105 of a binder support medium 90; the fluid communication between a conducting polymer component 10 and the detection zone 100 are as described above.

In the embodiment depicted in FIG. 5B an analyte 120 is complexed with a labeled receptor 110 by a covalent or noncovalent interaction to form an analyte-labeled receptor complex 115. The labeled receptor 110 comprises a receptor 114, which acts as a ligand for an analyte 120, and a label 112, the label being a reduction-oxidation catalyst; the two portions are joined by a covalent or noncovalent interaction. In a particular embodiment, the reduction-oxidation catalyst is a reduction-oxidation enzyme. In a particular embodiment the analyte-labeled receptor complex 115 is mobile, is formed upstream following application of a liquid sample containing analyte 120, and transported downstream in the direction of fluid flow 3 to the detection zone 100. As illustrated in FIG. 5C, the analyte-labeled receptor complex 115 may then be captured by a binder 130 in the detection zone 100 to form a relatively immobile labeled sandwich complex 55.

Also as depicted in FIG. 5D, the label 112 of the labeled sandwich complex 55 may catalyze the formation of a dopant molecule 104. Here the transformation of a first chemical product 84 and dopant precursor compound 101 to form a third chemical product 91 and dopant molecule 104, respectively, by catalytic conversions 89 and 103, respectively, are as described for FIG. 3C above. FIG. 5E illustrates diffusion 106 of dopant 104 into the conducting polymer component 10, resulting embedded dopant 107, the embedded dopant 109 imparts electrical conductivity to the conducting polymer component 10.

The binder 130 may be immobilized in the detection zone by covalent or ionic bonds, or as an insoluble or slowly diffusing compound, or alternatively the binder 130 may be mobile. The source of labeled receptor 110 and binder 130 within the device are important only to the extent that they accommodate control over the timing of formation of a sandwich complex 55 and its location in a detection zone 100, and to the extent that the device design minimizes premature catalytic conversion, for example upstream from the detection zone, of the dopant precursor compound 101 and the first chemical product 84. The dopant precursor compound 101 and first chemical product 84 are not confined to the stoichiometry of FIGS. 5A-5E: for signal amplification a substrate:enzyme stoichiometric ratio exceeding 1:1 would be needed for each of these compounds.

Methods for bonding receptors 114 to labels 112 comprised of a reduction-oxidation enzyme to obtain active conjugated labeled receptors 110 are known to those skilled in the art. The present device is not limited by the selection of a particular enzyme as a label. A variety of different labeled receptor 110 reagents can be formed by varying either the label 112 or the receptor 114 component of the labeled receptor 110; it will be appreciated by one skilled in the art that the choice involves consideration of the analyte to be detected and the amenability of label 112 and receptor 114 to chemical linking.

A sandwich configuration 55 may be independent of the assay protocol selected; however, the receptor 114 of the labeled receptor 110 may be dependent upon the assay protocol selected. For example, as indicated in FIG. 1, the protocol may be a sandwich type of assay, and the receptor 114 of the labeled receptor 110 can be capable of specifically binding with the analyte 120. Thus an analyte 120 may be an antigen, wherein an antibody specific for the antigen may be used as the receptor 114, or immunologically reactive fragments of the antibody, such as F(ab′)2, Fab or Fab′ may be used as the receptor 114. These receptors 114 coupled to the label 112 can then bind to an analyte 120 if present in the sample as the sample passes through a zone upstream of the detection zone 100, and form an analyte-labeled receptor complex 115 which can then be carried into the detection zone 100 on the binder support medium 90 by the fluid flow through the assay. When an analyte -labeled receptor complex 115 reaches the detection zone 100, it can be captured by a binder 130 which may optionally be an immobilized binder.

A receptor 114 can be a ligand or binder capable of complexing to an analyte of interest 120 or analogue or derivative thereof, forming a ligand-analyte complex. The terms receptor, ligand and binder are used interchangeably here. A ligand may specifically bind an aggregation of molecules; for instance the ligand may be an antibody raised against an immune complex of a second antibody and its corresponding antigen.

The choice of the receptor 114 of the labeled receptor 110 may depend upon the assay format. If the assay is a competitive assay, then the receptor 114 of the labeled receptor 110 may be chosen to couple reversibly and alternately with the analyte 120 and an analog thereof. If the assay format is a sandwich type, then the receptor portion 114 of the labeled receptor 110 may be a ligand which is bound specifically by the analyte 120 or alternatively by an antibody which is capable of binding specifically and simultaneously to the receptor 114 and the analyte 120.

The most common types of ligand-analyte complexes are antigen-antibody complexes. In certain embodiments, the analyte 120 can be an antigen, and the receptor 114 an antibody. In other embodiments, the analyte 120 can be an antibody, and the receptor 114 an antigen. Other ligand-analyte complexes capable of forming in the present invention may include specific binding pairs such as biotin and avidin, streptavidin and anti-biotin, carbohydrates and lectins, complementary nucleotide sequences, complementary peptide sequences, effector and receptor molecules, enzyme cofactors and enzymes, enzyme inhibitors and enzymes, a peptide sequence and an antibody specific for the sequence or the entire protein, polymeric acids and bases, dyes and protein binders, peptides and specific protein binders (e.g., ribonuclease, S-peptide and ribonuclease S-protein), metals and their chelators, and the like. Furthermore, ligand-analyte complexes can include members that are analogs of the original specific binding member, for example an analyte-analog or a specific binding member made by recombinant techniques or molecular engineering.

If the receptor 114 is an immuno-reactant it can be, for example, an antibody, antigen, hapten, or complex thereof. Antibodies useful for immunoassays of the present invention can include those specifically reactive with various analytes. Such antibodies may include IgG or IgM antibodies or mixtures thereof, which are essentially free of association with antibodies that are capable of binding with non-analyte molecules. The antibodies may be commercially available polyclonal or monoclonal antibodies, or may be obtained by ascites, tissue culture or other techniques known in the art. The use of mixtures of monoclonal antibodies of differing antigenic specificities or of monoclonal antibodies and polyclonal antibodies may be desired. The invention contemplates that fragments of antibody molecules may be used as specific binding reagents, including half antibody molecules and Fab, Fab′ or F(ab′)2 fragments known in the art. In one embodiment the antibodies may be generally free of impurities. The antibodies may be purified by column chromatography or other conventional means; in some instances it may be desirable to purify by known affinity purification techniques.

Antigens and haptens useful in carrying out the immunoassays of the present invention may include materials, whether natural or synthesized, which present antigenic determinants for which the analyte antibodies are specifically reactive when presented on the chromatography medium of the invention. Synthetic antigens can include antigens made by chemical syntheses as well as antigens made by recombinant DNA techniques. Antigens may also be labeled with detectable particles for use in sandwich type assays to detect of antibody analytes or in competition assays to detect antigen analytes.

Compounds suitable for use as binder 130 are available, and include the same classes of compounds that can be employed as receptor 114. Their choice varies with the analyte of interest. Examples of particularly suitable binders 130 include but are not limited to antibodies directed to an analyte of interest or directed to a homologue or derivative of an analyte of interest.

The detection zone 100 on the binder support medium 90 comprises a binder 130, capable of binding an analyte-labeled receptor complex 115. The invention contemplates use of reduction-oxidation catalysts, including reduction-oxidation enzymes, possessing sufficiently fast kinetics that it would be unnecessary to immobilize binder 130 on detection zone 100. Where in certain embodiments a binder 130 is immobilized at a detection zone 100 in a competitive assay, by “immobilized,” it is meant that the binder 130, once on the binder support medium 90, may not be capable of substantial movement to positions elsewhere within the binder support medium 90. Thus, an analyte-labeled receptor complex 115 may be captured at a detection zone 100 by a binder 130.

The free or immobilized binder 130 of the invention may include any molecule or compound that capable of binding the analyte 120, analyte-labeled receptor complex 115, labeled receptor 110, or similar detectable complex. For example, the binder 130 may consist of any ligand capable of binding the analyte-labeled receptor complex 115. As an example, an antibody may be used as the binder 130. Suitable specific binding reagents for the sandwich complex 55 are known by those of ordinary skill in the art and are generally readily identifiable.

Because the binder support medium 90 may be chemically inert, it can be activated at the detection zone 100 as necessary to immobilize a specific binder 130. The method of immobilizing a binder 130 depends on the chemical properties of the binder support medium 90. Examples of direct immobilization methods may include adsorption, absorption and covalent binding such as by use of (i) a cyanogen halide, e.g., cyanogen bromide or (ii) by use of glutaraldehyde. Other methods may include treatment with Schiff bases and borohydride for reduction of aldehydic, carbonyl and amino groups. DNA, RNA and certain antigens may be immobilized against solvent transport by baking onto the binder support medium 90.

Depending on the assay, it may be preferred, however, to retain or immobilize a binder 130 on a binder support medium 90 indirectly through the use of insoluble micro- or nano-particles to which the binder 130 is attached. Methods generally known by those of ordinary skill in the art for attaching a reagent to micro- or nano-particles encompass both covalent and non-covalent mechanisms including adherence, absorption, or adsorption. For example, micro- and nano-particles can be selected from any suitable particulate polystyrene, polymethylacrylate, polyacrylamide, polypropylene, latex, polytetrafluoroethylene, polyacrylonitrile, polycarbonate, glass or similar material.

A binder 130 can be deposited on or in a detection zone 100 singly or in various combinations or configurations as are generally known by one of ordinary skill in the art, to produce different detection or measurement formats.

FIGS. 5A-5E also illustrates the use of an optional control zone 108 to confirm various results in a detection zone 100. A control zone 108 may provide a visual indication of wetting, i.e., confirming that a liquid sample has run its course. For example, a control zone 108 may comprise an anhydrous reagent that undergoes a color change when wetted, e.g., anhydrous copper sulphate wetted in a control zone 108 may turn blue. A control zone 108 may be located adjacent to or within a detection zone 100.

The control zone 108 may also be designed to indicate whether the fluid sample has reached that point, and whether performance conditions of an assay are within acceptable ranges for the assay. Thus a control zone 108 may comprise a pH indicator dye such as phenolphthalein, which changes from clear to intense pink upon contact with a solution whose pH is in the range of 8.0-10.0, a common range for assay fluids. Similarly, a control zone 108 may comprise immobilized analyte 120 to capture excess labeled receptor 110 that has not bound to an analyte upstream; the captured labeled receptor 110 in the control zone 108 in combination with substrate reagents can confirm results observed in a detection zone 100.

In one embodiment the control zone 108 may host the complexation of a dopant precursor compound 101 and first chemical product 84, such that uncomplexed labeled receptor 110 from upstream could react and be quantified in the control zone 108. Thus an additional conducting polymer component in fluid communication with the control zone 108 could serve as a second readout circuit to confirm results of a first readout circuit that detects dopant generation in the detection zone.

Types of control zone strategies suitable for use in the embodiments as described herein are not limited to the use of any particular control or control strategy.

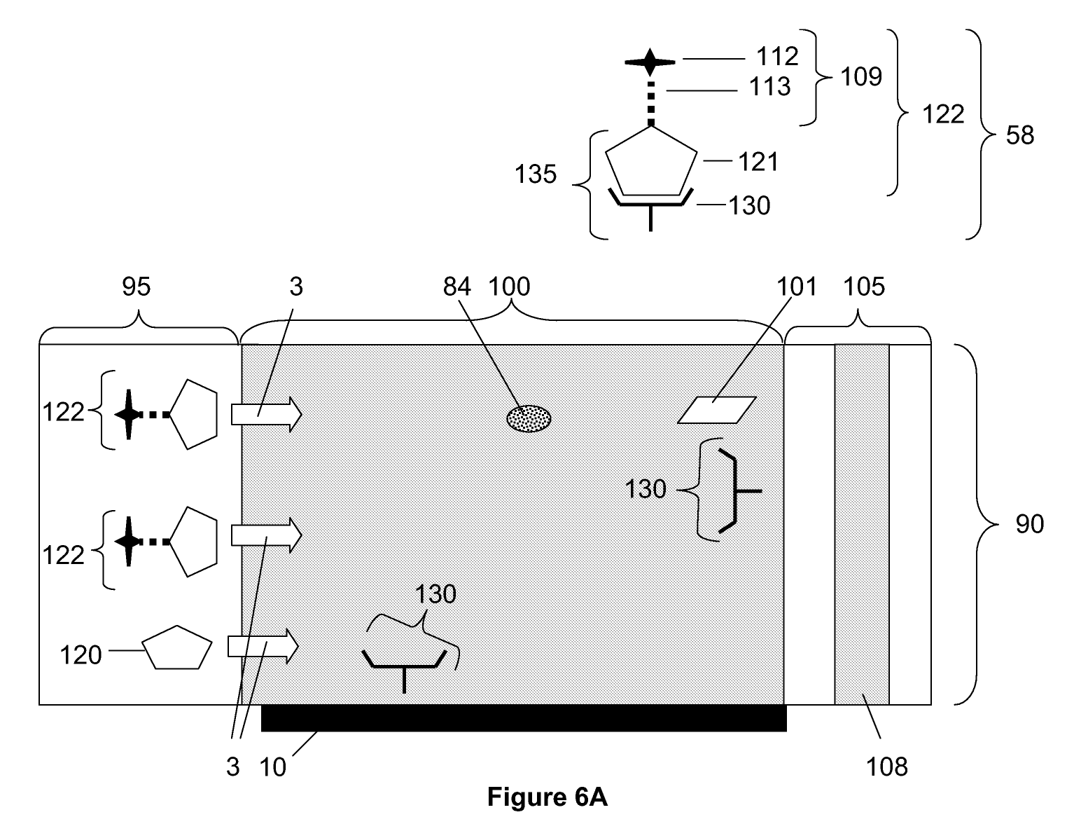

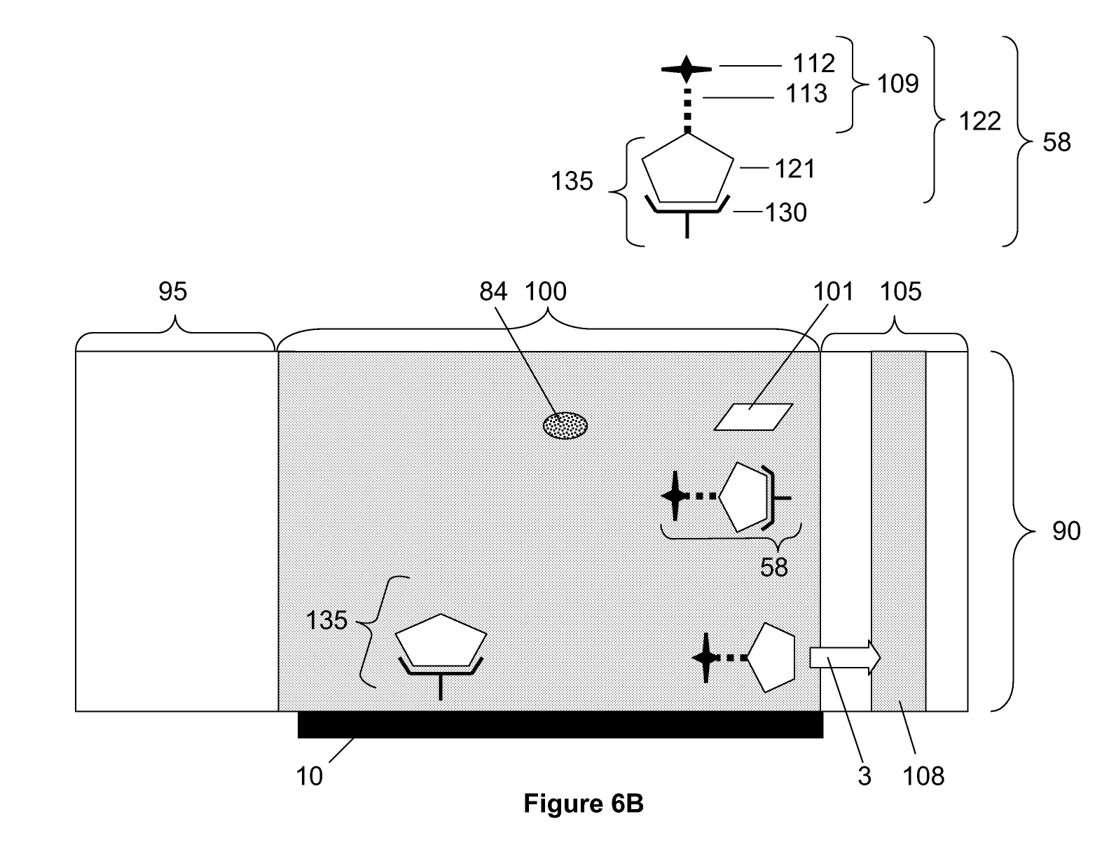

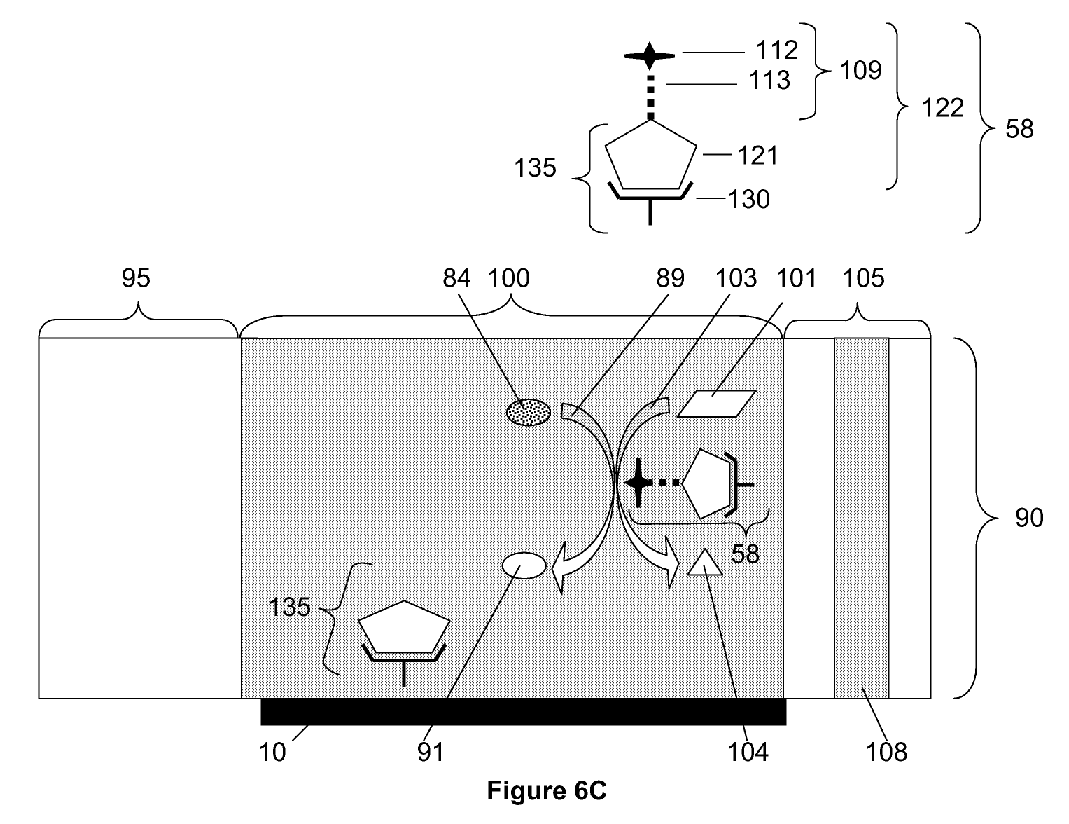

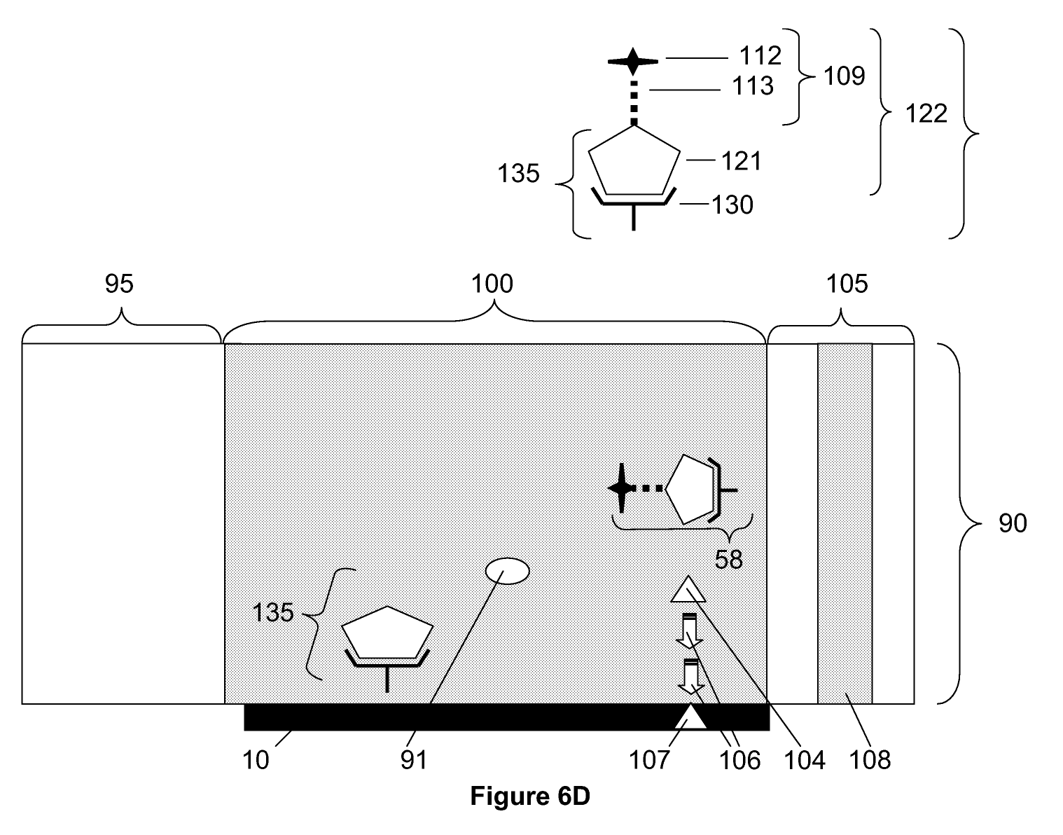

FIGS. 6A-6D are schematics illustrating one embodiment of an immunoassay, comparable to FIG. 5A-5E, but illustrating some general features for a system that provides a one-enzyme competitive assay—as opposed to a sandwich assay—that may generate a chemical detection event in the device. In FIGS. 6A-6D the application of a liquid sample and its direction of fluid flow 3 though a first portion 95, detection zone 100, and third portion 105 and its control zone 108 of a binder support medium 90 are as described above, as is the fluid communication between a conducting polymer component 10 and the detection zone 100. Also as described above for FIG. 5A-5E are the binder 130, first chemical product 84. dopant precursor molecule 101, third chemical product 91, dopant molecule 104, and catalytic conversions 89 and 103. The novel feature of FIGS. 6A-6D is an competitor complex 122 comprised of: a competitor 121 that may be an analyte or an analog or derivative of an analyte; a label 112, the label being a reduction-oxidation catalyst, such as a reduction-oxidation enzyme; and a linking portion 113 which may be a bond or a chemical moiety such as an alkyl chain, peptide chain, or other linker that is bonded to each of the competitor 121 and the label 112. The label 112 and the linking portion 113 together comprise a pendant catalyst moiety 109. The competitor complex 122 upon association with a binder 130 may form a competitive-binder complex 58. The label 112 of the competitor-binder complex 122 may act upon a dopant precursor compound 101 and a first chemical product 84 in a manner that is comparable to a single enzyme sandwich assay according to the present invention as illustrated in FIGS. 5A-5E.

In an alternative embodiment, a binder 130 or other binder may be located upstream of the detection zone 100 to capture analyte from a liquid sample, such that the analyte 120 competes with competitor complex 122 for complexation to the upstream binder sites. Thus more molecules of competitor complex 122 are expected to be transported downstream in the direction of fluid flow 3 when analyte is present in a sample, because the competitor complex 122 is less likely to be trapped by upstream binder. Where analyte 120 that is not trapped upstream washes downstream into the detection zone 100, it may be captured by a binder 130. Competitor complex 122 that is not captured by a binder upstream or by a binder 130 may be expected to be washed downstream past the detection zone 100 into the third portion 105 of the binder support medium 90, including into the control zone 108 wherein the competitor complex 122 may optionally be detected in control zone 108 as in a second detection zone to confirm the results obtained in the first detection zone 100.

In an alternative embodiment, label 112 may be coupled to a receptor 114 that is competitive with an analyte 120. Both analyte 120 from the sample and competitor complex 122 may progress with the flow of liquid sample to a detection zone 100, and may then react with the binder 130 found on the detection zone 100. Unlabeled analyte 120 may thus reduce the amount of competitor complex 122 captured in detection zone 100, in which case the signal strength in detection zone 100 scales inversely with the amount of analyte.

The same considerations that govern in the choice of enzyme, receptors, linkers, and binders, as well as the control zone, for the embodiment illustrated in FIGS. 5A-5E also apply for the embodiment illustrated in FIGS. 6A-6E.

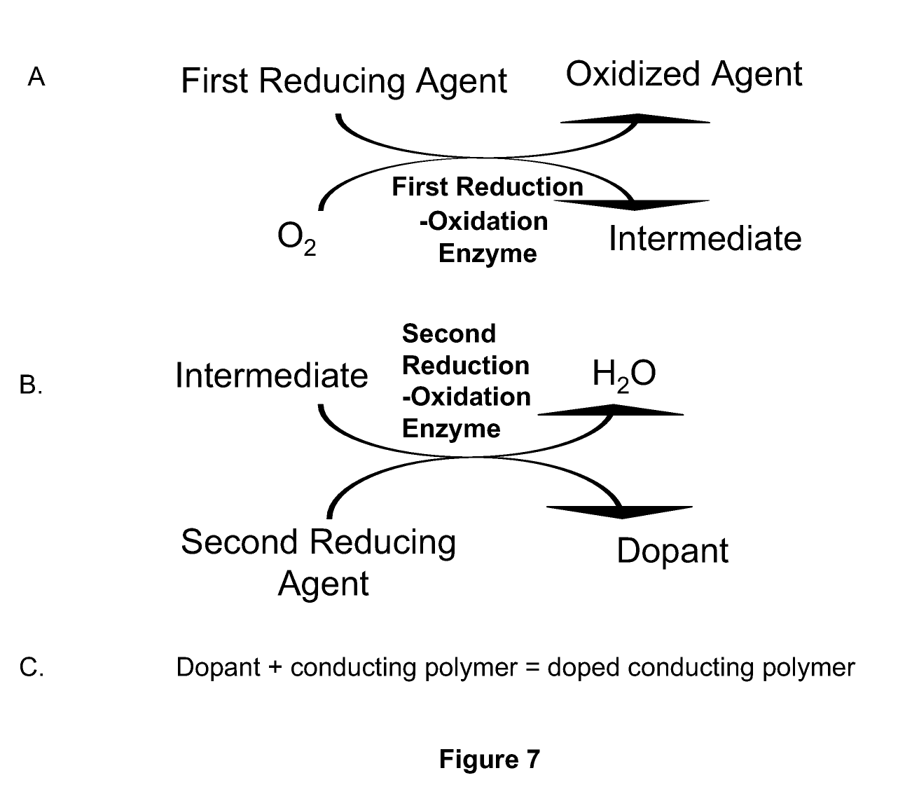

FIG. 7 is a schematic illustrating one embodiment of an immunoassay that utilizes a two-enzyme strategy to generate a dopant. Steps B and C of FIG. 7 correspond to the two steps of FIG. 2, but with a clarification that byproduct is water; thus a one-enzyme immunoassay strategy is similar to the second catalytic reaction of a two-enzyme immunoassay strategy. Step A of FIG. 7 is an inverse analog of Step B: in step A the reducing agent is oxidized to a byproduct, and the desired product is obtained through the reduction half reaction instead of the oxidation half reaction. In certain embodiments, the first reduction-oxidation enzyme or, alternatively, the second reduction-oxidation enzyme is bound to the analyte or analyte competitor.

It should be understood that the invention is not limited by the details depicted in FIG. 2 and FIG. 7. A reduction-oxidation enzyme necessarily requires the simultaneous occurrence of two half reactions, because an intermolecular transfer of electrons or atoms is involved: one half reaction has a net loss of electrons of atoms; the other half reaction has a net gain. Likewise transferase enzymes may require parallel half reactions. However it is anticipated that dopant generation by means of enzymes that catalyze cleavage or combination of molecules without such intermolecular transfers would result in simplified equations.

First reduction-oxidation enzymes suitable for the present example may include but are not limited to enzymes that catalyze the production of an oxidized intermediate (such as hydrogen peroxide, an organic peroxide, a disulfide, or a quinone) from the reaction of an oxidizer (such as dioxygen, a quinone, or sodium hypochlorite) with a reducing agent (such as diiodide, glucose or another sugar, a thiol, a phenol, or glutathione reductase); examples of such enzymes include glucose oxidase, alcohol:NAD⁺ reduction-oxidation enzyme, lactase enzymes, and others. Non-limiting examples of first reduction-oxidation enzymes contemplated by the invention include dehydrogenases, oxidases, lactases, peroxidases and catalases.

The first reducing agent and second reducing agent may be compounds capable of donating one or more electrons, hydrogen atoms, hydrides or hydride equivalents to an oxidizing compound. Examples of suitable first and second reducing agents include but are not limited to iodide anion, glucose and other sugars, thiols, sulfide salts, phenols, phenoxide salts, and glutathione reductase.

Examples of second reduction-oxidation enzymes suitable for this embodiment can include but are not limited to enzymes that catalyze the production of a dopant such as triiodide anion from the reaction of an oxidized intermediate (such as hydrogen peroxide, an organic peroxide, a disulfide, or a quinone) with a reducing agent (such as diiodide or glutathione reductase). Second reduction-oxidation enzymes contemplated by the invention can include but are not limited to dehydrogenases, oxidases, lactases, peroxidases and catalases. The second reduction-oxidation enzyme may be bound to or merely contained unbound within the binder support medium, or may be bound to or contained unbound within an element such as a reagent pad, or may be bound to or localized within the conducting polymer component. Specific examples of such enzymes include but are not limited to lactose peroxidase, horseradish peroxidase, and similar enzymes.

In alternative embodiments, the second reduction-oxidation enzyme may be bound to the analyte or analyte competitor, and the first reduction-oxidation enzyme may be located in a bound or unbound state in the detection zone.

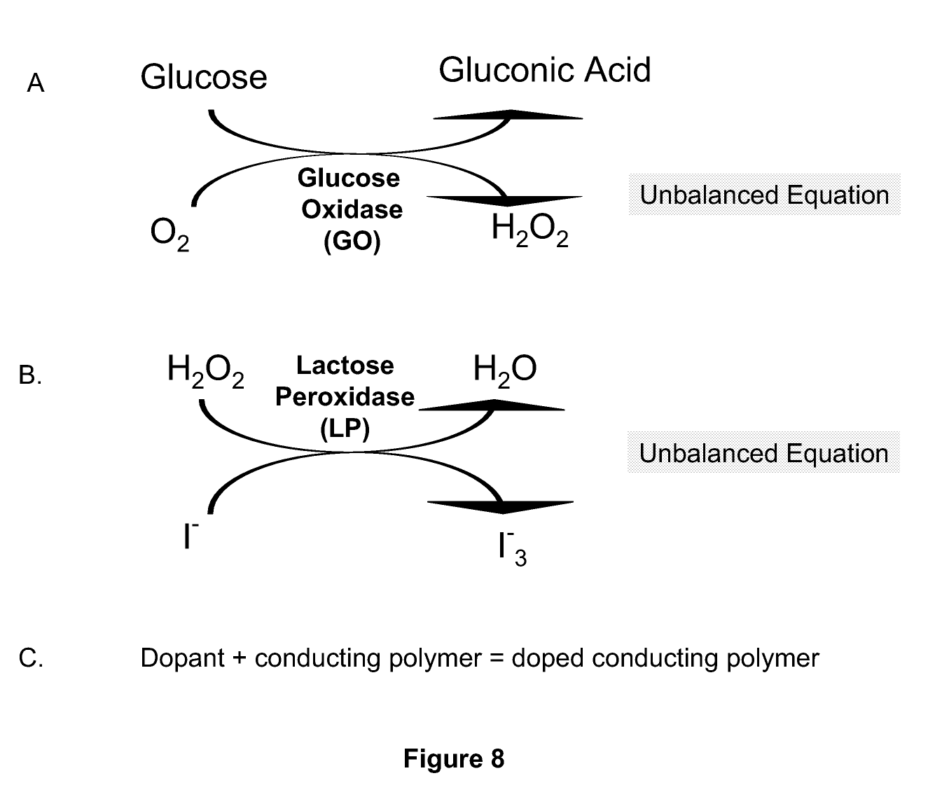

FIG. 8 is a schematic illustrating a particular embodiment of an immunoassay for a two-enzyme system; it may be understood to illustrate the chemistry of FIG. 7 in greater detail. In step A glucose is oxidized (i.e., here it has hydrogen atoms extracted) to gluconic acid, thereby serving as a source of hydrogen atoms (i.e., a reducing agent) whereby molecular dioxygen may be catalytically converted to the intermediate hydrogen peroxide by the reduction oxidation enzyme glucose oxidase. In step B the intermediate hydrogen peroxide is converted to water, which is the more reduced entity of the two, and thus this half reaction acts as a sink for the two electrons that are extracted during the conversion of iodide anion to triiodide anion by the reduction oxidation enzyme lactose peroxidase. Step C is as in FIG. 7.

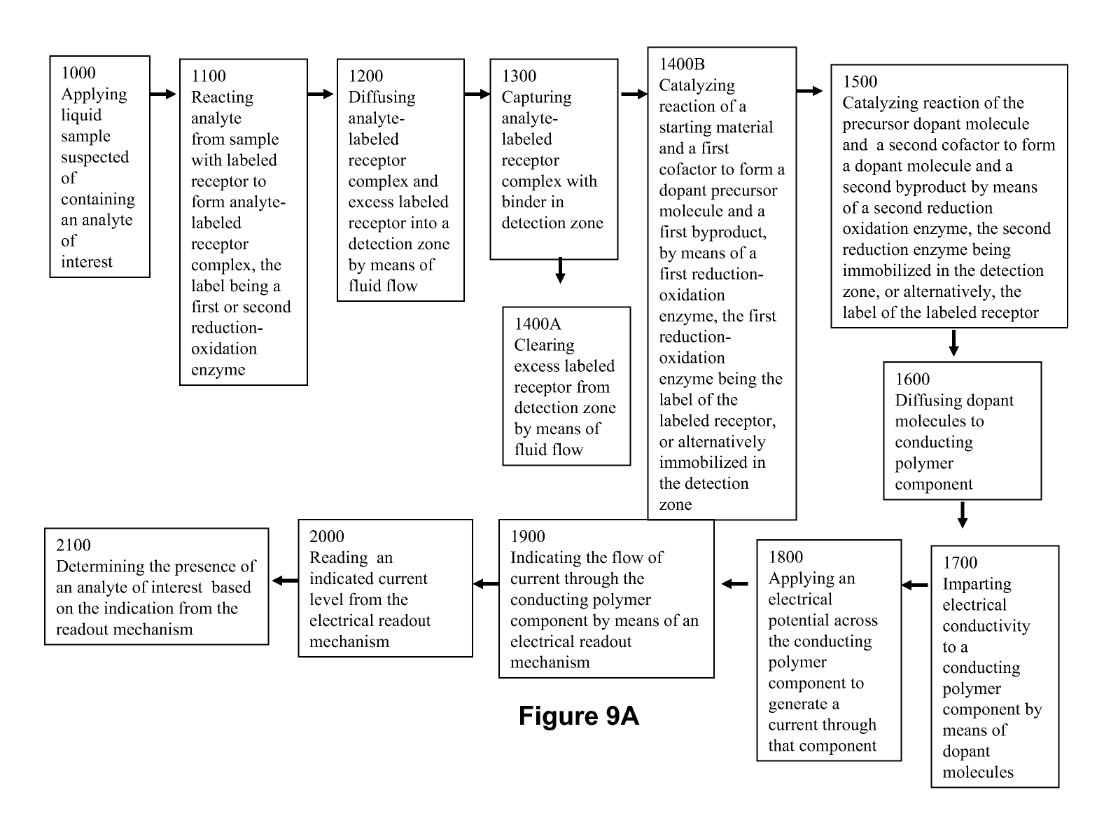

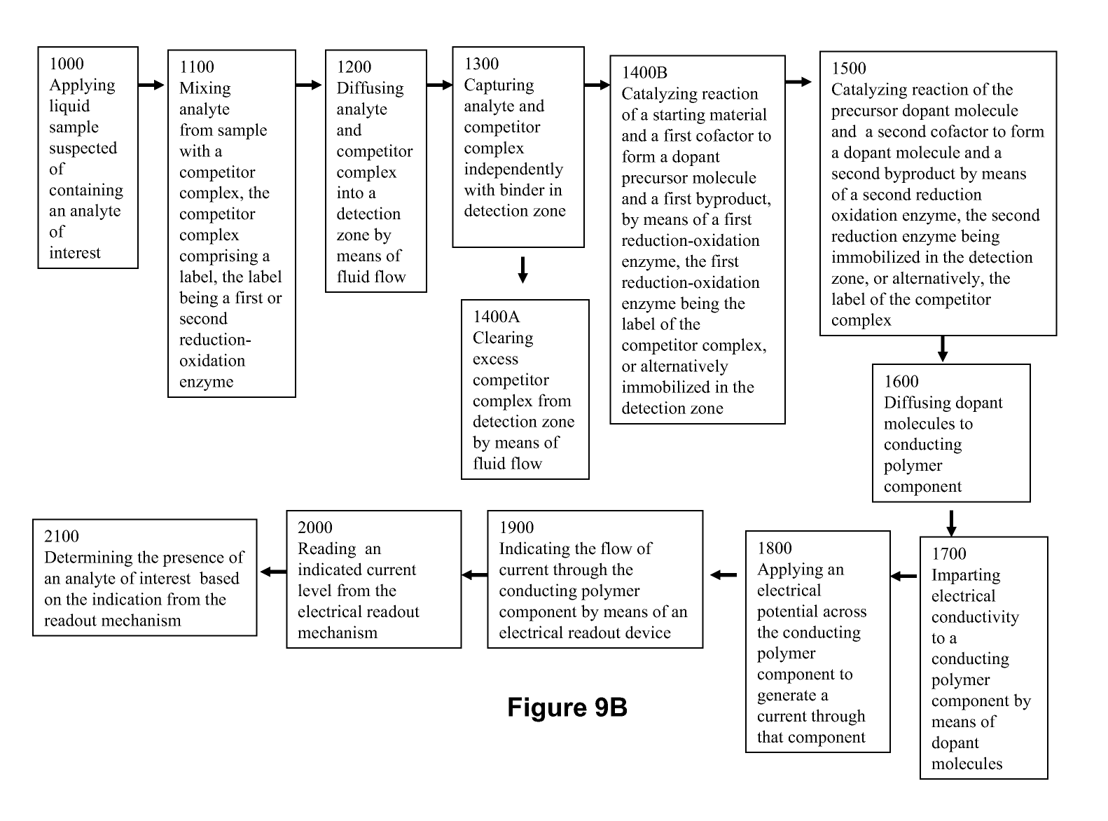

FIG. 9A is a flow chart illustrating one embodiment of a sandwich immunoassay for a two-enzyme system; it may be understood to illustrate chronologically an example of the assay sequence. In step 1000, apply a liquid sample suspected of containing an analyte of interest to the assay. In step 1100, react the analyte, if present in the liquid sample, with a labeled receptor to form an analyte-labeled receptor complex. The label of the labeled receptor is a reduction-oxidation enzyme. The reduction-oxidation enzyme may be a first reduction-oxidation enzyme or a second reduction-oxidation enzyme, depending on the particular strategy the assay employs. For example, the label may be a first reduction-oxidation enzyme that participates in a first catalysis reaction in a serial catalysis reaction that ultimately results in the formation of a dopant. If the label is a first reduction-oxidation enzyme, then the second reduction-oxidation enzyme that participates in a second catalysis reaction in a serial catalysis reaction will be contained in the detection zone. Alternatively, the label may be a second-oxidation enzyme, and the first oxidation-reduction enzyme may be contained in a detection zone. In a particular embodiment, the first reduction-oxidation enzyme may be a glucose oxidase enzyme. In a particular embodiment, the second reduction-oxidation enzyme may be a lactose peroxidase enzyme. The labeled receptor may be present in dry form in the assay, and capable of dissolving in the liquid sample at the time of application. In step 1200, diffuse the analyte-labeled receptor complex into a detection zone by means of fluid flow. Because the sample is a liquid, the sample's liquid may be exploited to provide the fluid flow of the immunoassay. In step 1300, capture the analyte-labeled receptor complex with immobilized binder molecules which immobilize the complex in the detection zone. In step 1400A, clear any unbound labeled receptor from the detection zone by means of fluid flow. The excess labeled receptor is washed downstream from the detection zone. In step 1400B, catalyze a starting material and a first cofactor to form a dopant precursor molecule and a first byproduct, by means of a first reduction-oxidation enzyme. The first reduction-oxidation enzyme can be the label of the labeled receptor, or contained in the detection zone, as described in step 1100. In step 1500, catalyze the precursor molecule and a second cofactor to form a dopant molecule by means of a second reduction-oxidation enzyme. The second reduction-oxidation enzyme can be contained in the detection zone, or the label of the labeled receptor, as described in step 110.

In step 1600, diffuse the dopant molecule produced in the catalysis reaction to a conducting polymer component. In step 1700, impart electrical conductivity to a conducting polymer component by doping the polymer with the diffused dopant molecule produced in the catalysis reaction. The doping of the polymer allows an electrical potential to pass through the conducting polymer. In step 1800, apply an electrical potential applied across the conducting polymer component to generate an electrical current through the conducting polymer component. The applied potential may be one that is held constant prior to and throughout the assay of the liquid sample, or may be applied at one or more convenient times during the assay. In step 1900, indicate the flow of current through the conducting polymer by means of an electrical readout mechanism. The amount of electrical current that is transmitted by the conducting polymer component can be detected and indicated by the electrical readout mechanism. In step 2000, read the indication level from the electrical readout mechanism. In step 2100, determine the presence of an analyte based on the indication from the readout mechanism.

FIG. 9B is a flow chart illustrating one embodiment of a competitive immunoassay for a two-enzyme system; it may be understood to illustrate chronologically an example of the assay sequence. In step 1000, apply a liquid sample suspected of containing an analyte of interest to the assay. In step 1100, mix the analyte from the sample with a competitor complex contained in the assay device. The competitor complex is a competitor comprising an analyte, analogue, or derivative thereof that is incapable of binding to the analyte of interest, but is capable of competing with the analyte of interest from the sample in binding an immobilized binder in the detection zone. The competitor complex further comprises a label, the label being a reduction-oxidation enzyme. The reduction-oxidation enzyme may be a first reduction-oxidation enzyme or a second reduction-oxidation enzyme, depending on the particular strategy the assay employs. For example, the label may be a first reduction-oxidation enzyme that participates in a first catalysis reaction in a serial catalysis reaction that ultimately results in the formation of a dopant. If the label is a first reduction-oxidation enzyme, then the second reduction-oxidation enzyme that participates in a second catalysis reaction in a serial catalysis reaction will be contained in the detection zone. Alternatively, the label may be a second-oxidation enzyme, and the first oxidation-reduction enzyme may be contained in a detection zone. In a particular embodiment, the first reduction-oxidation enzyme may be a glucose oxidase enzyme. In a particular embodiment, the second reduction-oxidation enzyme may be a lactose peroxidase enzyme. The competitor complex may be present in dry form in the assay, and capable of dissolving in the liquid sample at the time of application. In step 1200, diffuse the analyte and the competitor complex into a detection zone by means of fluid flow. Because the sample is a liquid, the sample's liquid may be exploited to provide the fluid flow of the immunoassay. In step 1300, capture the analyte and the competitor complex with immobilized binder molecules which immobilize the analyte and complex in the detection zone. The competitor complex and analyte are both capable of binding to the immobilized binder in the detection zone. Thus, the analyte and the competitor complex compete for the binder. In step 1400A, clear any non-immobilized competitor complex from the detection zone by means of fluid flow. The excess competitor complex is washed downstream from the detection zone. In step 1400B, catalyze a starting material and a first cofactor to form a dopant precursor molecule and a first byproduct, by means of a first reduction-oxidation enzyme. The first reduction-oxidation enzyme can be the label of the competitor complex, or contained in the detection zone, as described in step 1100. In step 1500, catalyze the precursor molecule and a second cofactor to form a dopant molecule by means of a second reduction-oxidation enzyme. The second reduction-oxidation enzyme can be contained in the detection zone, or the label of the competitor complex, as described in step 1100.

In step 1600, diffuse the dopant molecule produced in the catalysis reaction to a conducting polymer component. In step 1700, impart electrical conductivity to a conducting polymer component by doping the polymer with the diffused dopant molecule produced in the catalysis reaction. The doping of the polymer allows an electrical potential to pass through the conducting polymer. In step 1800, apply an electrical potential applied across the conducting polymer component to generate an electrical current through the conducting polymer component. The applied potential may be one that is held constant prior to and throughout the assay of the liquid sample, or may be applied at one or more convenient times during the assay. In step 1900, indicate the flow of current through the conducting polymer by means of an electrical readout mechanism. The amount of electrical current that is transmitted by the conducting polymer component can be detected and indicated by the electrical readout mechanism. In step 2000, read the indication level from the electrical readout mechanism. In step 2100, determine the presence of an analyte based on the indication from the readout mechanism.

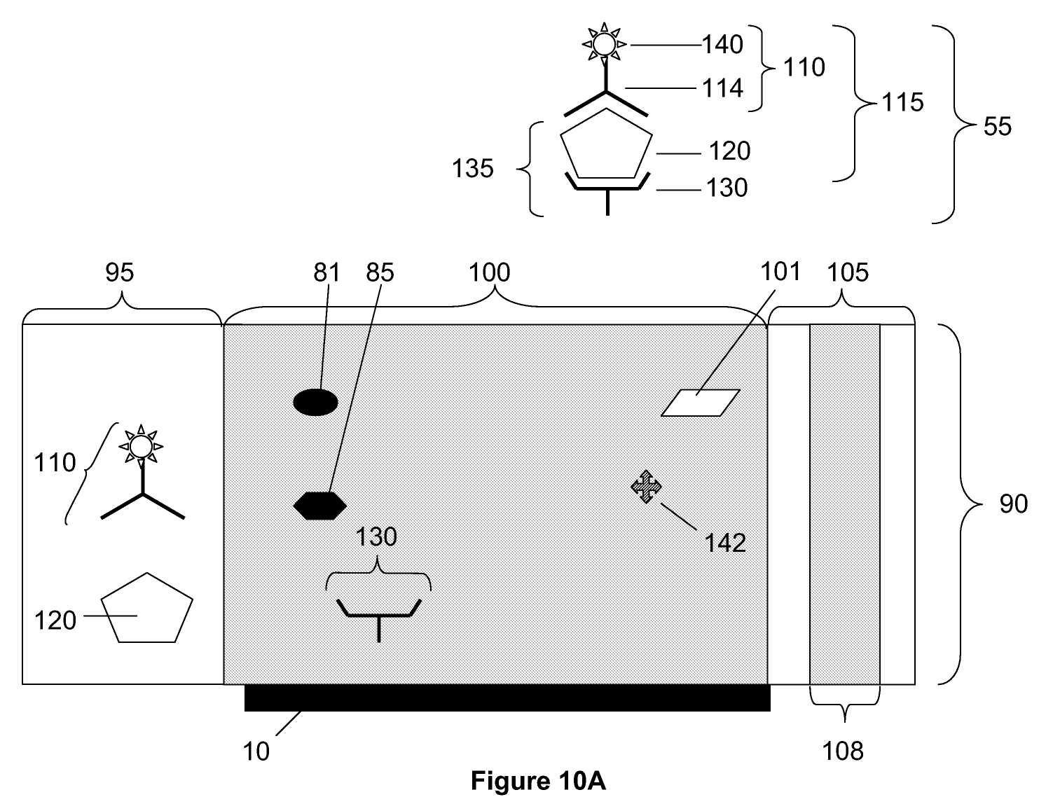

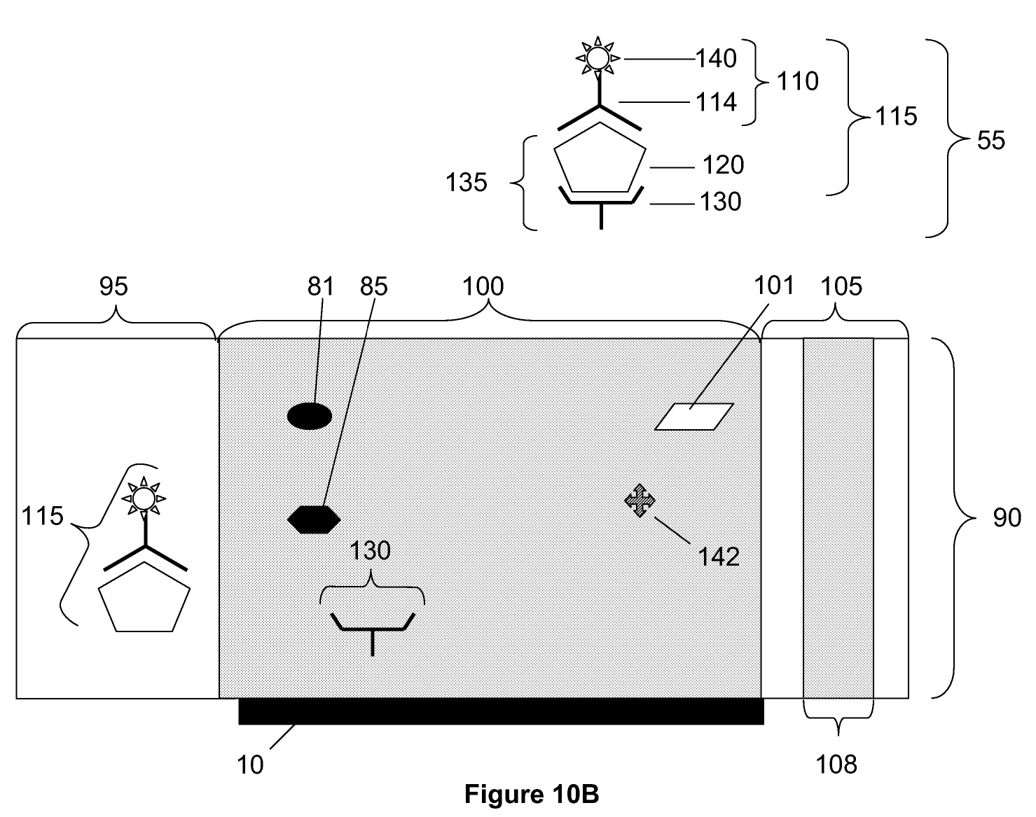

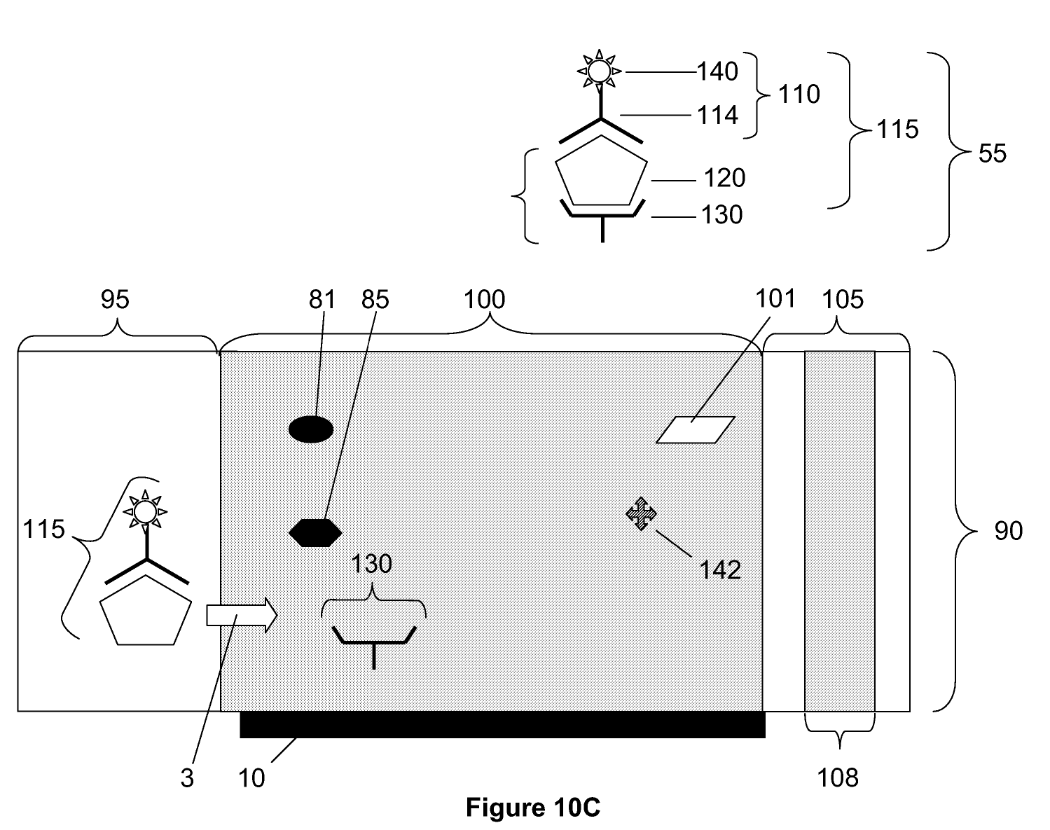

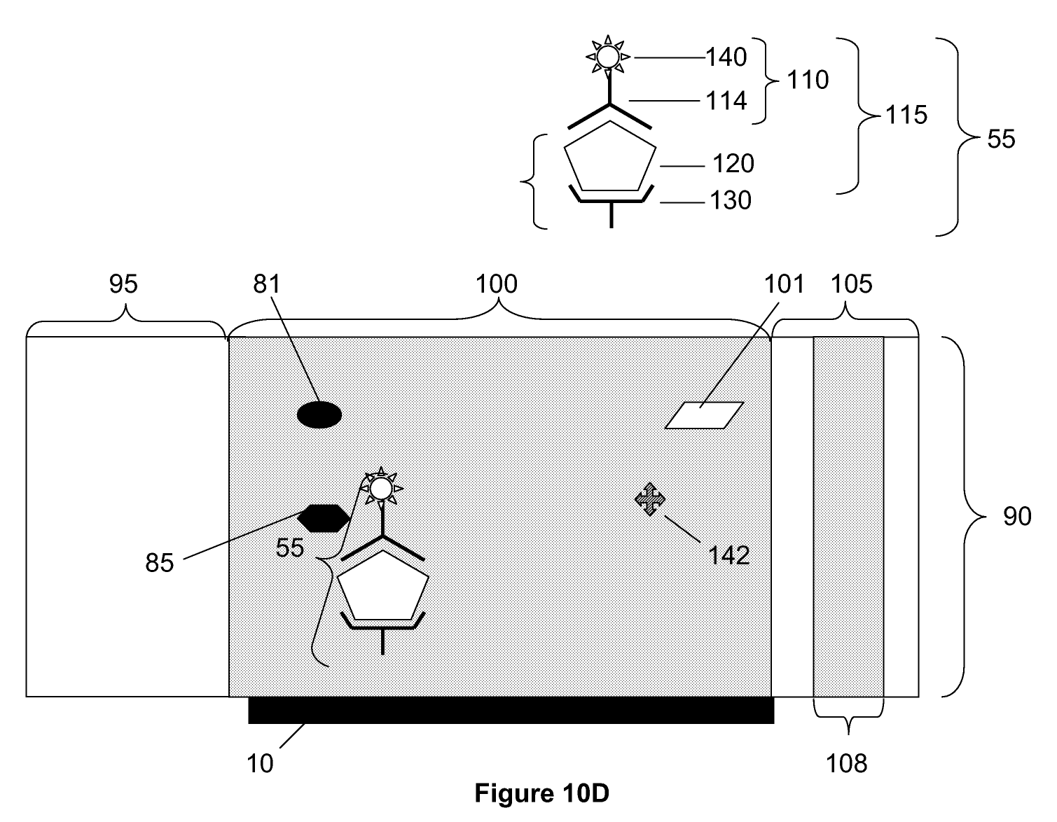

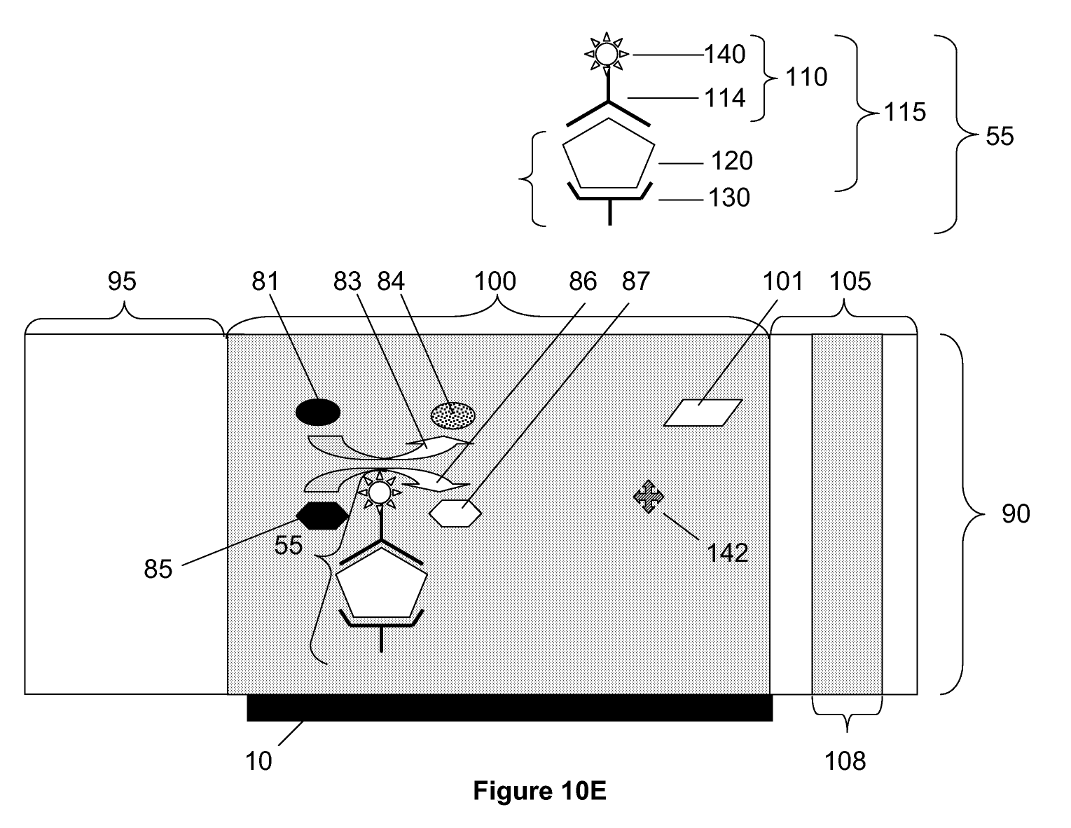

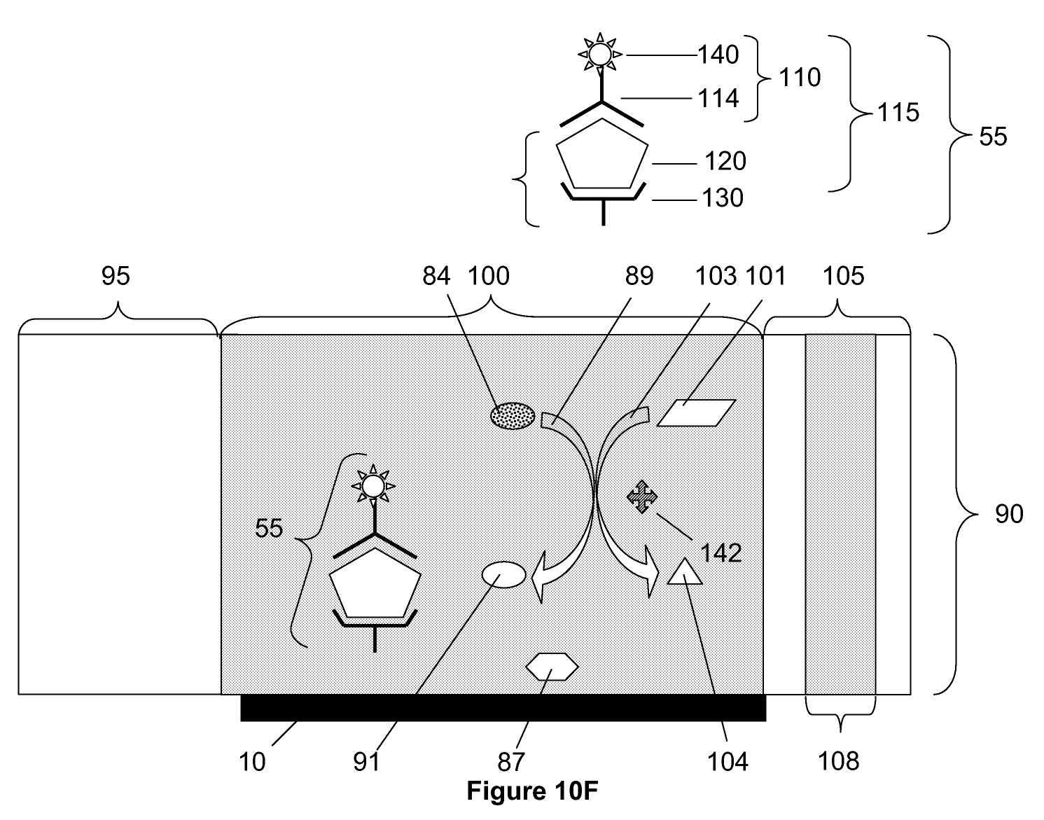

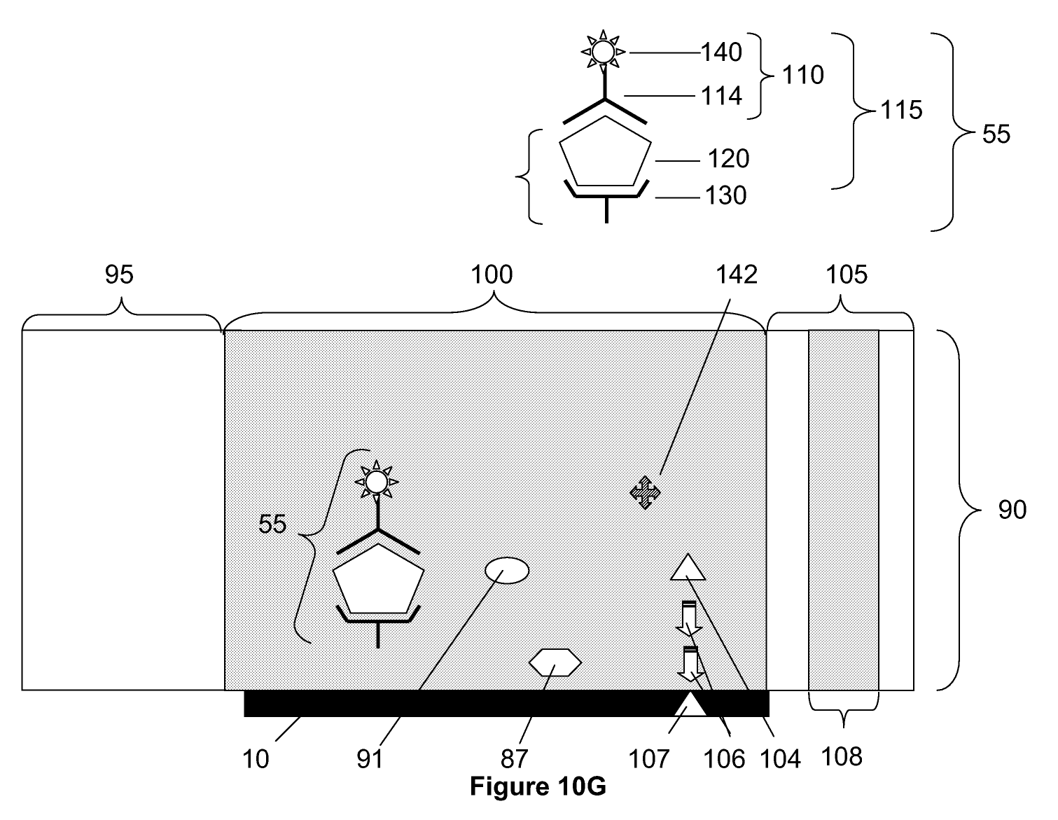

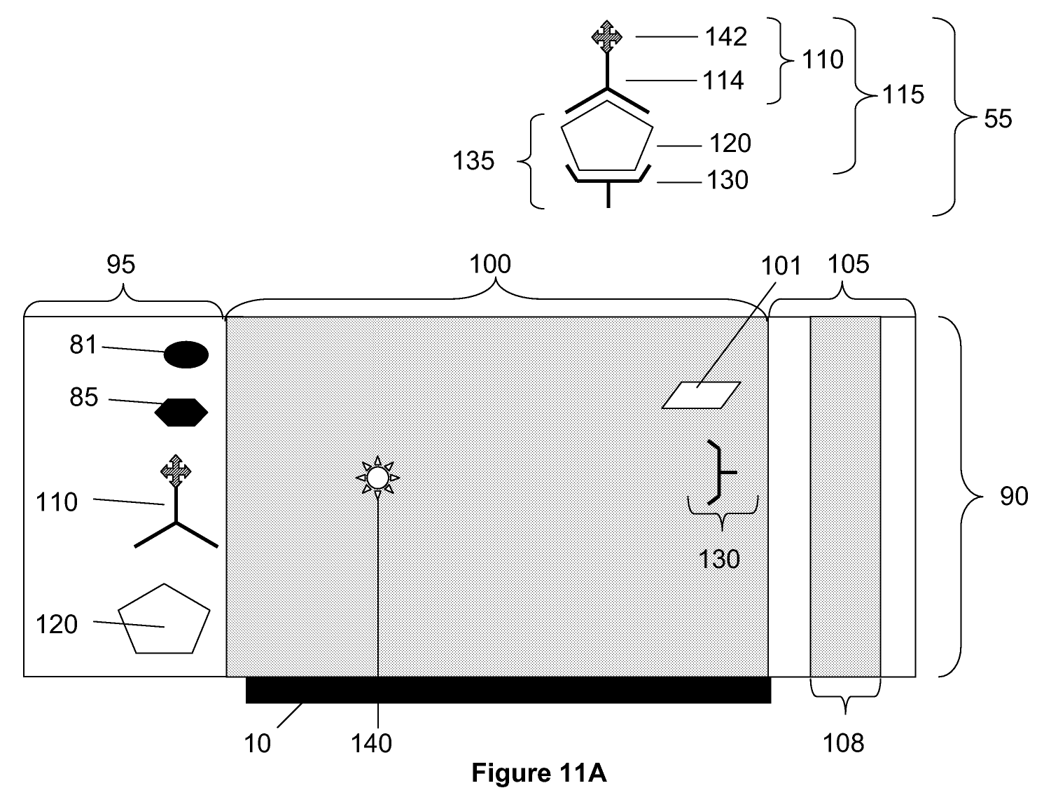

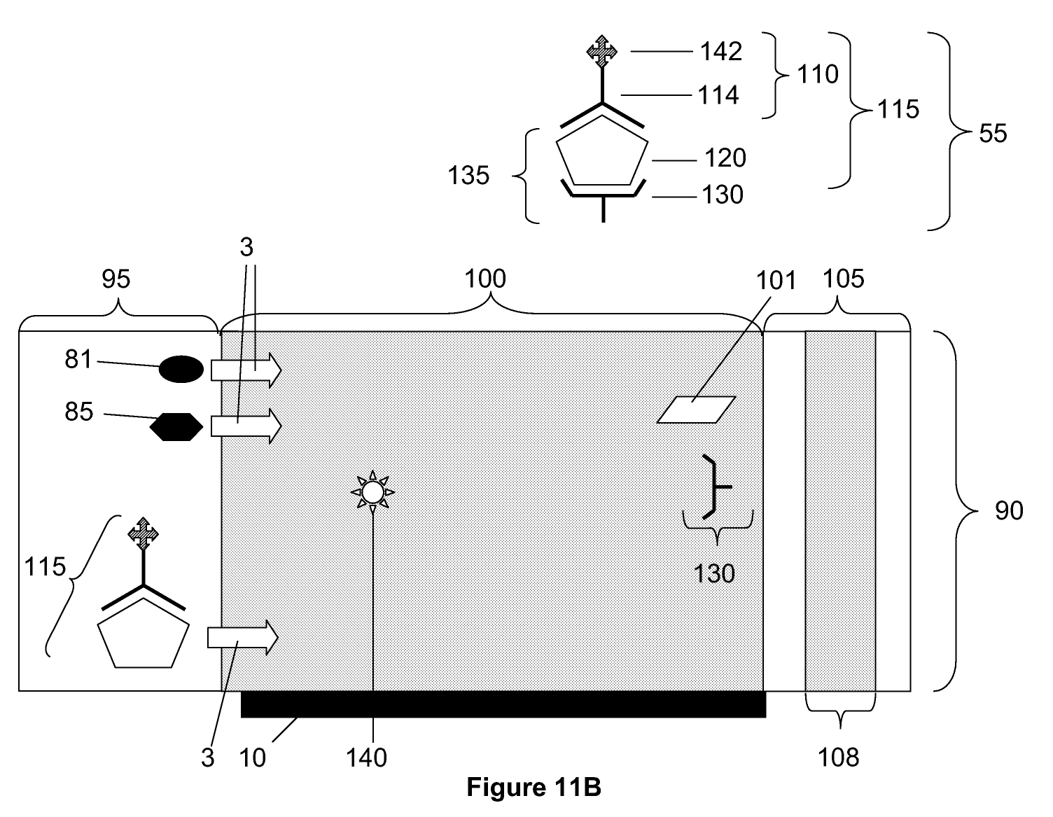

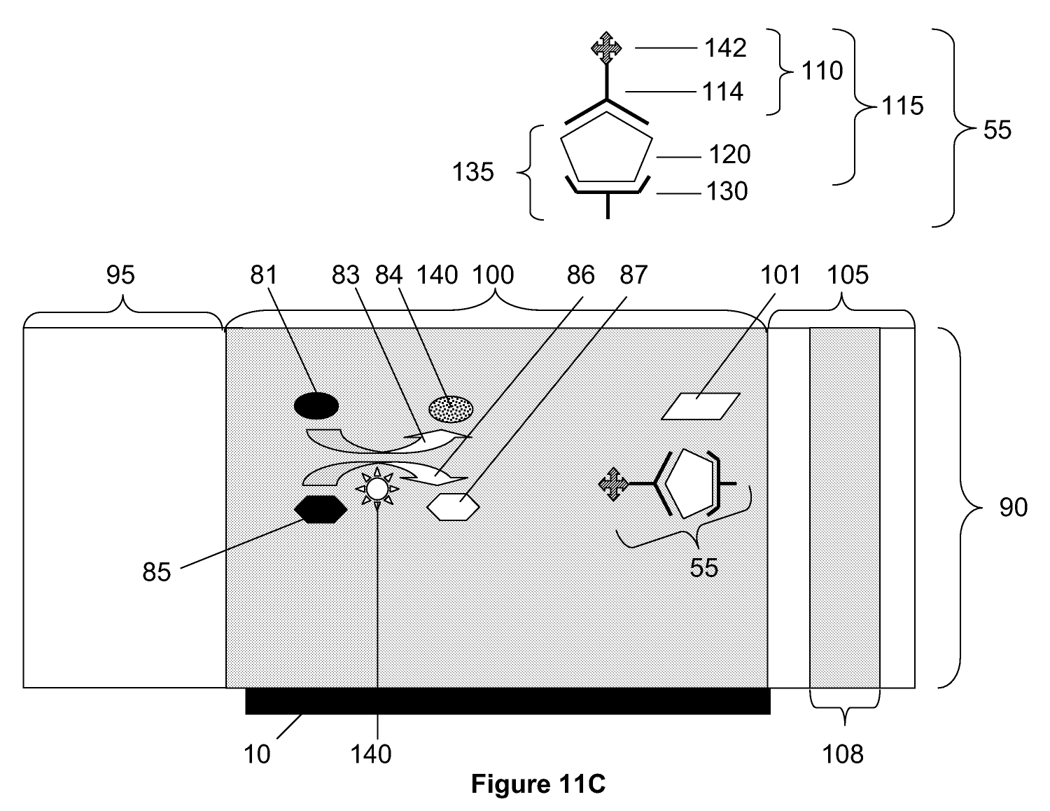

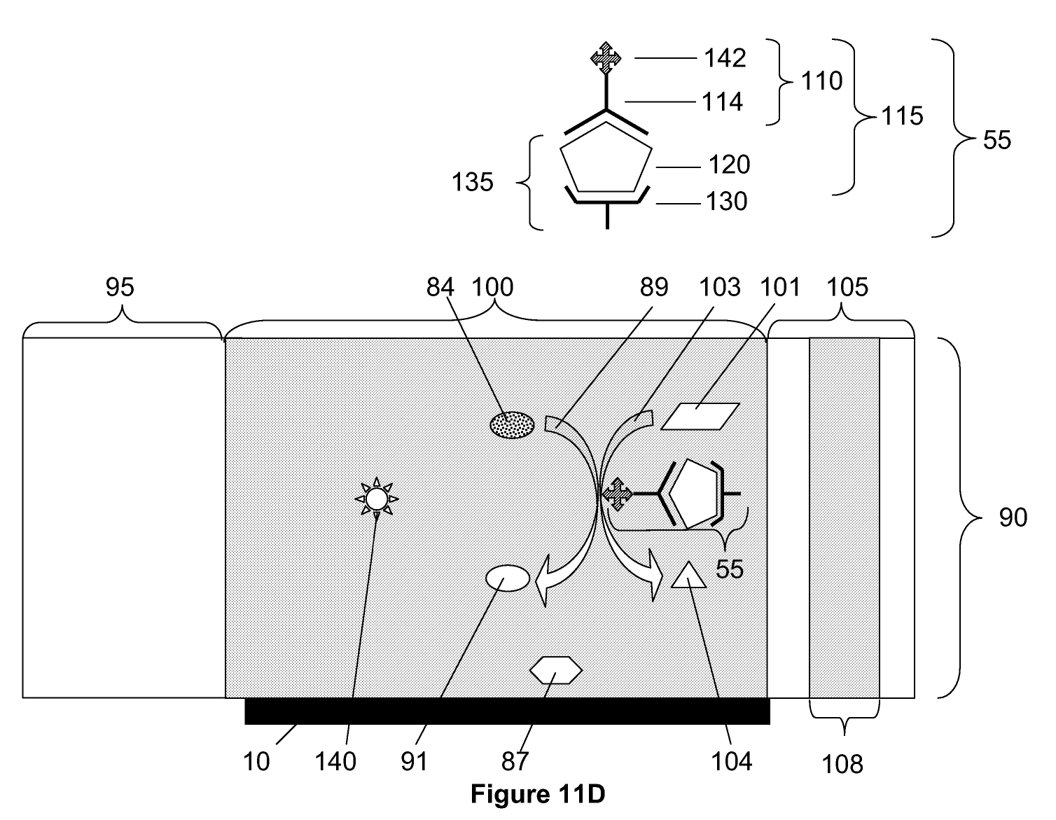

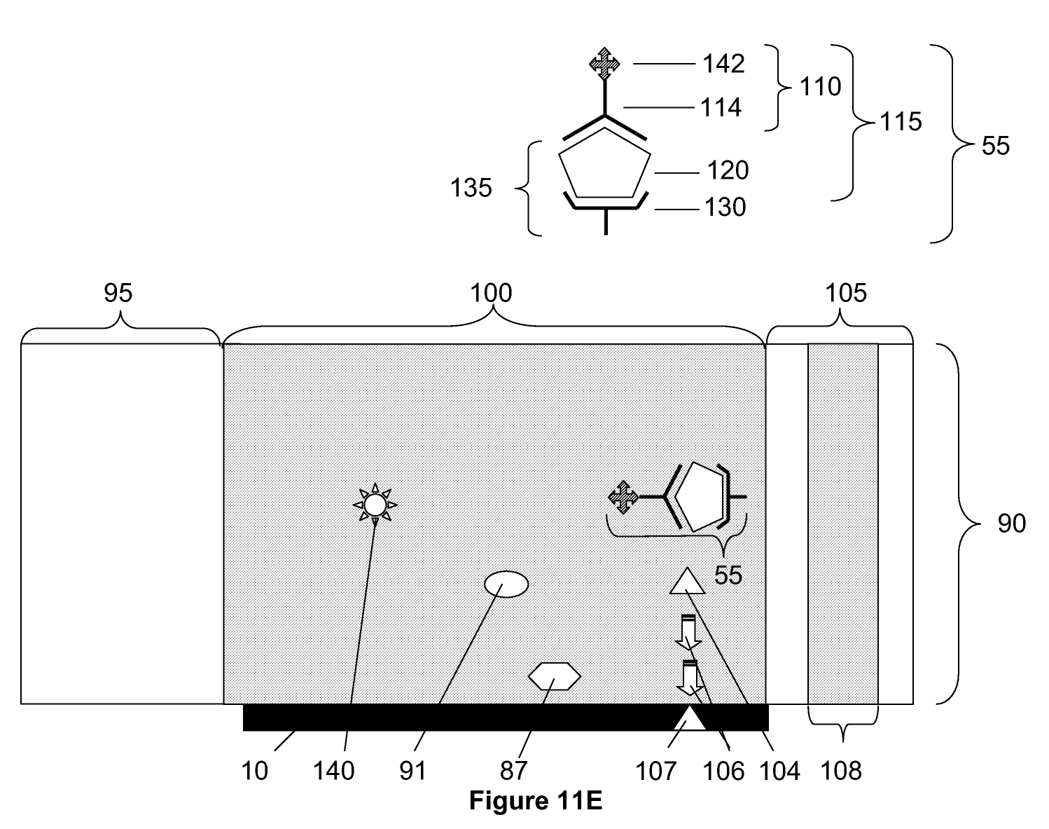

FIGS. 10A-10G and 11A-11E are schematics illustrating two respective embodiments of an immunoassay; comparable to FIGS. 5A-5E, but illustrating some general features for a system that provides details of the use of a two enzyme strategy in a sandwich assay that may generate a chemical detection event in the device. These two embodiments are completely analogous except that in FIGS. 10A-10G a first reduction-oxidation enzyme 140 in a two-enzyme catalytic series is part of a labeled receptor 110 wherein a second reduction-oxidation enzyme 142 is not, and in FIGS. 11A-11E a first reduction-oxidation enzyme 140 in a two-enzyme catalytic series is not part of a labeled receptor 110 wherein a second reduction-oxidation enzyme 142 is part of a labeled receptor 110.

FIG. 10A illustrates the initial state of an assay, before significant binding of the analyte 120 by a labeled receptor 110 in a first portion 95 of the binder support medium 90. A second reduction-oxidation enzyme 142 is located in the detection zone 100 of the binder support medium 90. A first chemical starting compound 81, second chemical starting compound 85, and dopant precursor compound 101 are provided in the detection zone 100, wherein a binder 130 is also located.

In an alternative embodiment, the second reduction-oxidation enzyme 142 can be located in the first portion 95 of the binder support medium 90.

FIG. 10B depicts the combination of the analyte 120 and labeled receptor 110 to form an analyte-labeled receptor complex 115, and FIG. 10C depicts the direction of fluid flow 3, which carries the analyte-labeled receptor complex 115 into the detection zone 100. As depicted in FIG. 10D the analyte receptor complex 115 may be captured by the binder 130 in the detection zone 100, generating a sandwich complex 55. As depicted in FIG. 10E in a first catalytic reaction the first reduction oxidation enzyme 140 of the sandwich complex 55, may catalyze the transformation of the first chemical starting compound 81 and second chemical starting compound 85 to form a first chemical product 84 and second chemical product 87, respectively, by means of catalytic conversions 83 and 86, respectively. As depicted in FIG. 10F, a complementary second reduction-oxidation enzyme 142, located in the detection zone, may then act upon the first chemical product 84 and dopant precursor 101 to produce a third chemical product 91 and dopant molecule 104, respectively, by means of conversions 89 and 103, respectively. As depicted in FIG. 10G, the dopant molecule 104 may undergo a diffusion 106 that transports it onto the surface of or into the mass of a conducting polymer component 10, resulting in an embedded dopant 107. The embedded dopant 107 imparts electrical conductivity to the conducting polymer component 10.

FIGS. 11A-11E are analogous to FIGS. 10A-10G As depicted in FIG. 11A, at the outset of the assay the first portion 95 of a binder support membrane 90 may contain an analyte 120, first chemical starting compound 81, second chemical starting compound 85, and a labeled receptor 110 in which the label is a second reduction-oxidation enzyme 142; the detection zone 100 may contain a first reduction-oxidation enzyme 140, dopant precursor 101, and binder 130. As depicted in FIG. 11B the labeled receptor 110 and analyte 120 may combine to form an analyte-labeled receptor complex 115, with diffusion in the direction of fluid flow 3 carrying the complex into the detection zone 100.

As depicted in FIG. 11C the analyte-labeled receptor complex 115 may be captured by the binder 130 in the detection zone 100, generating a sandwich complex 55. Also depicted in FIG. 11C in a first catalytic reaction a first reduction-oxidation enzyme 140 may catalyze the transformation of the first chemical starting compound 81 and second chemical starting compound 85 to form a first chemical product 84 and second chemical product 87, respectively, by means of catalytic conversions 83 and 86, respectively. As depicted in FIG. 11D, a second reduction-oxidation enzyme 142 that comprises the label portion of a labeled receptor 110, may then act upon the first chemical product 84 and dopant precursor 101 to produce a third chemical product 91 and dopant molecule 104, respectively, by means of conversions 89 and 103, respectively. As depicted in FIG. 11E, the dopant molecule 104 may undergo a diffusion 106 that transports it onto the surface of or into the mass of a conducting polymer component 10, resulting in an embedded dopant 107. The embedded dopant 107 imparts electrical conductivity to the conducting polymer component 10.

It an alternative embodiment, the first chemical starting compound 81 and a second chemical starting compound 85 are chemically isolated from, for instance in a different location from, the first reduction-oxidation enzyme 140 that is capable of acting directly upon them in the catalytic series. Thus, FIGS. 11A-11G may alternatively be configured such that at the outset of the assay the labeled receptor 110 is located in the detection zone 100, and the first chemical starting compound 81 and second chemical starting compound 85 are located in the first portion 95 of the binder support medium 90. In a further alternative embodiment, the assay depicted in FIGS. 11A-11E may alternatively be configured such that the first reduction-oxidation enzyme 140 is located in the first portion 95 of the binder support medium 90 and the first chemical starting compound 81 and second chemical starting compound 85 are located in the detection zone 100.

For either the competitive or sandwich assays with two enzymes in series, at the outset of the assay the dopant precursor 101 may be located in the first portion 95 or detection zone 100 of the binder support medium 90, if the dopant precursor 101, provided that the first reduction-oxidation enzyme 140 is separated from the first and second chemical starting compounds 81 and 85, respectively.

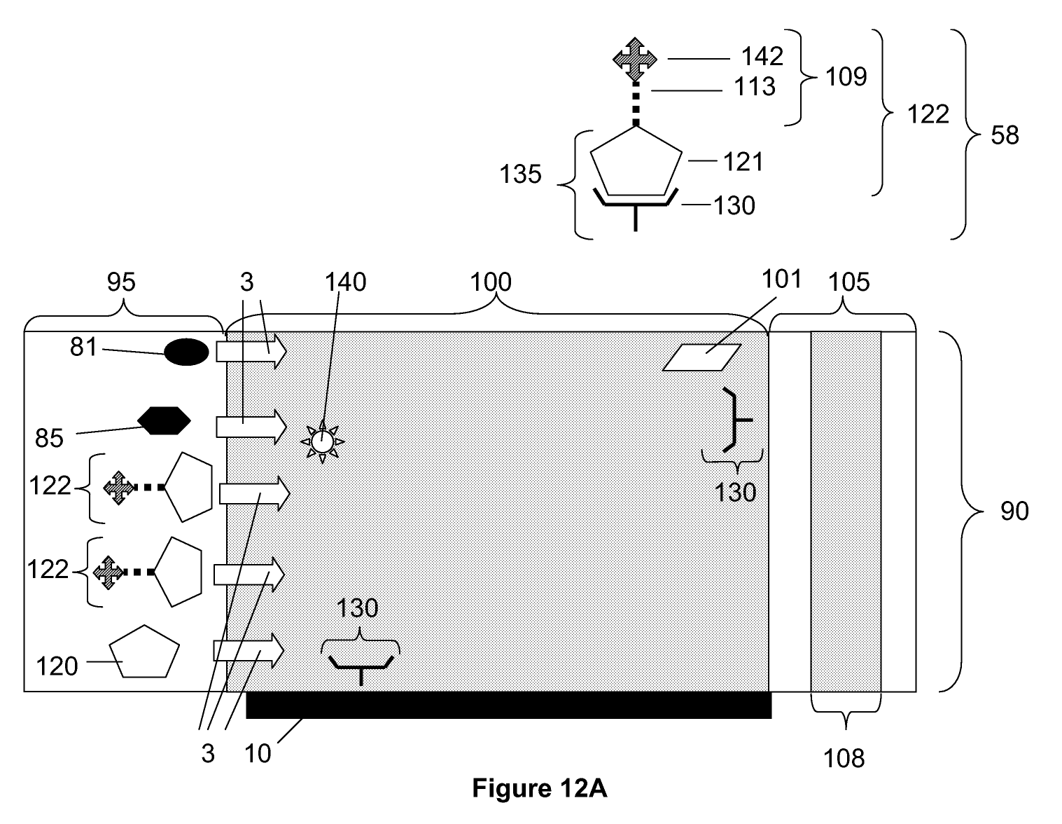

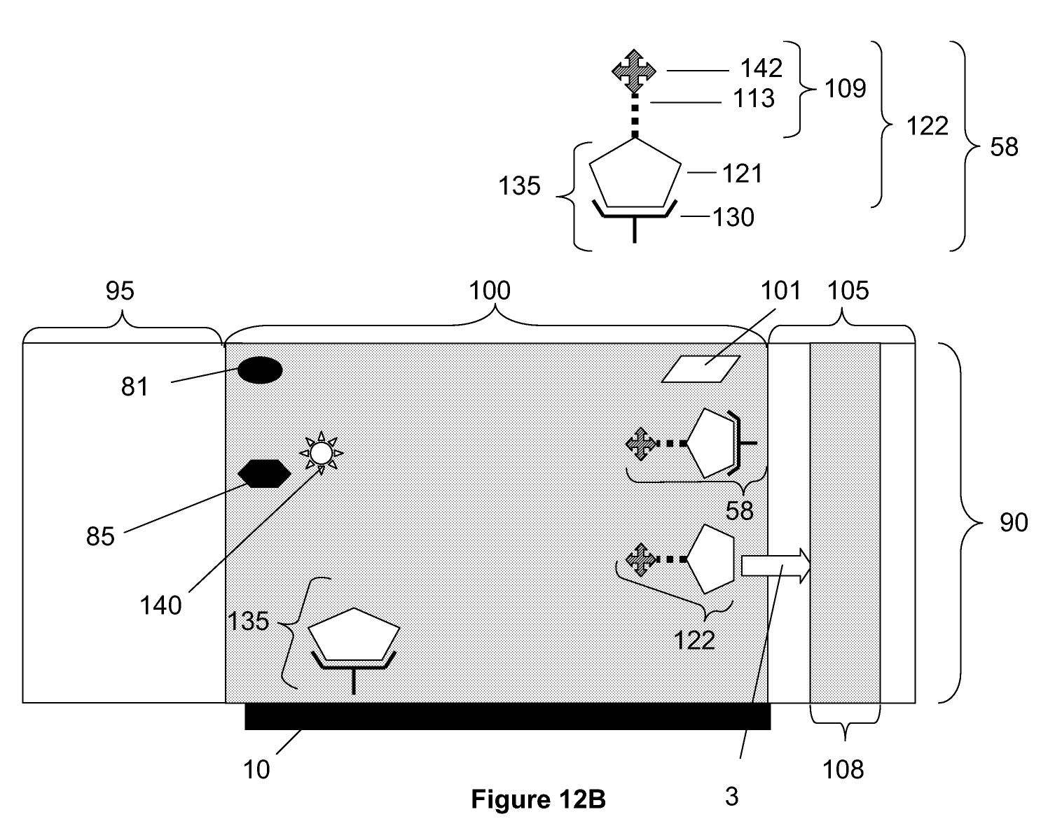

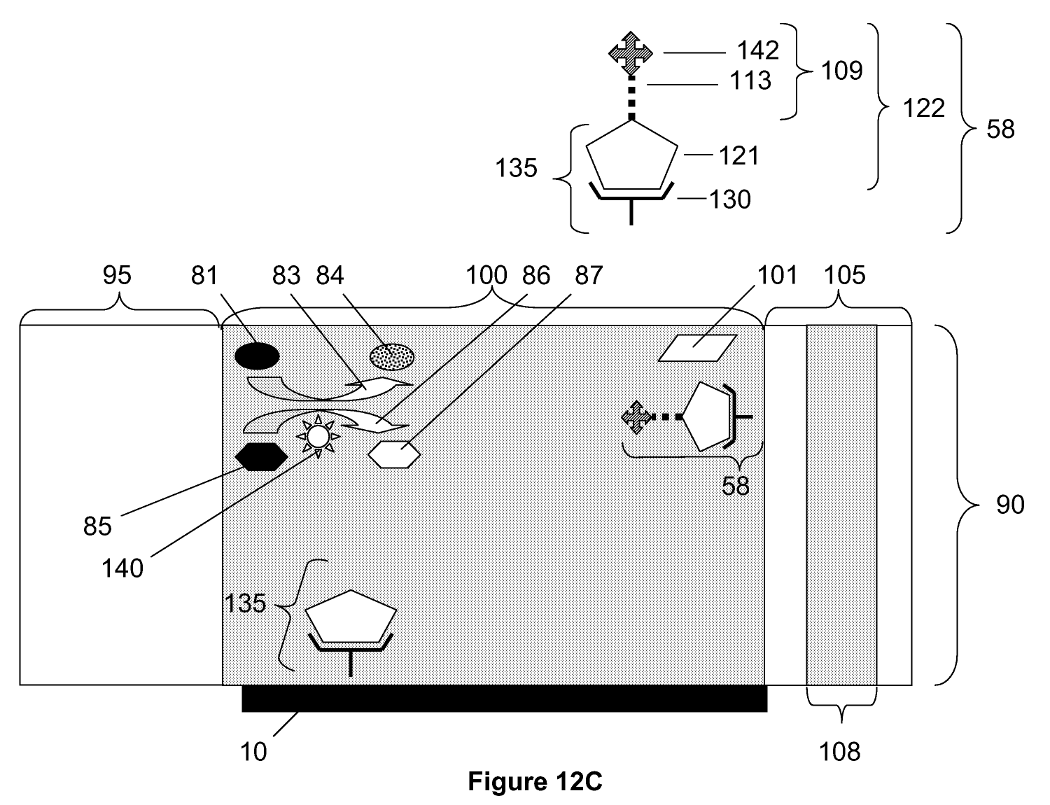

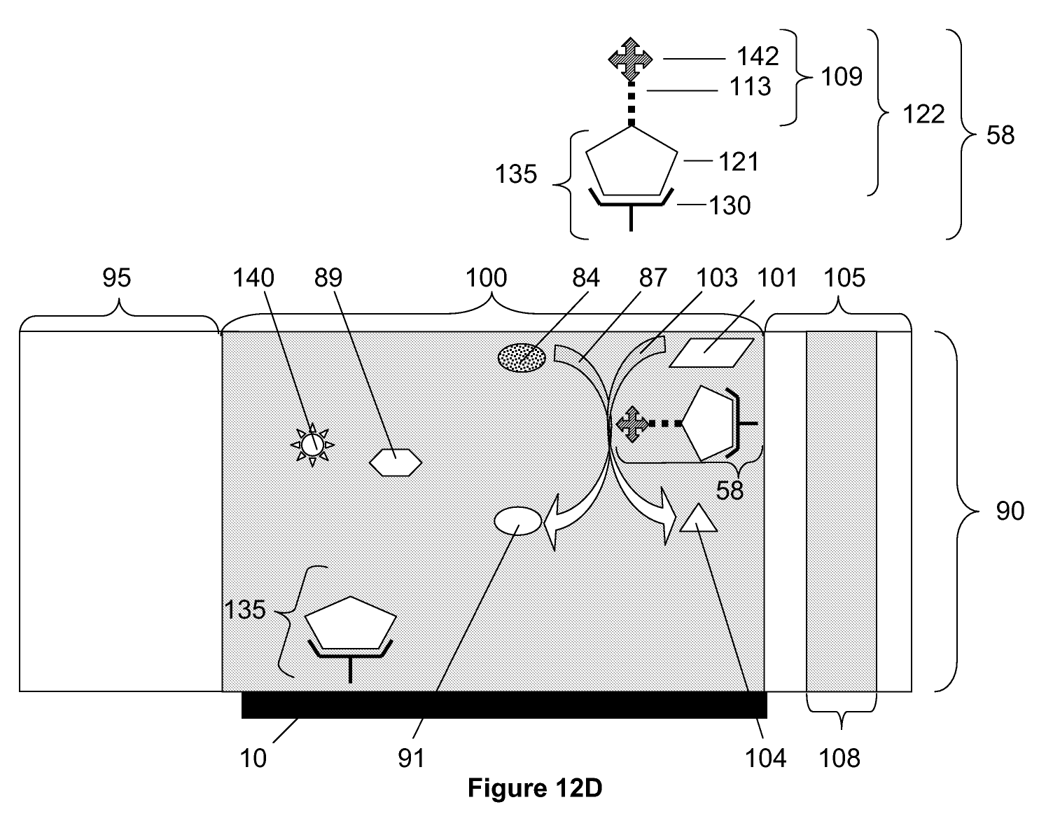

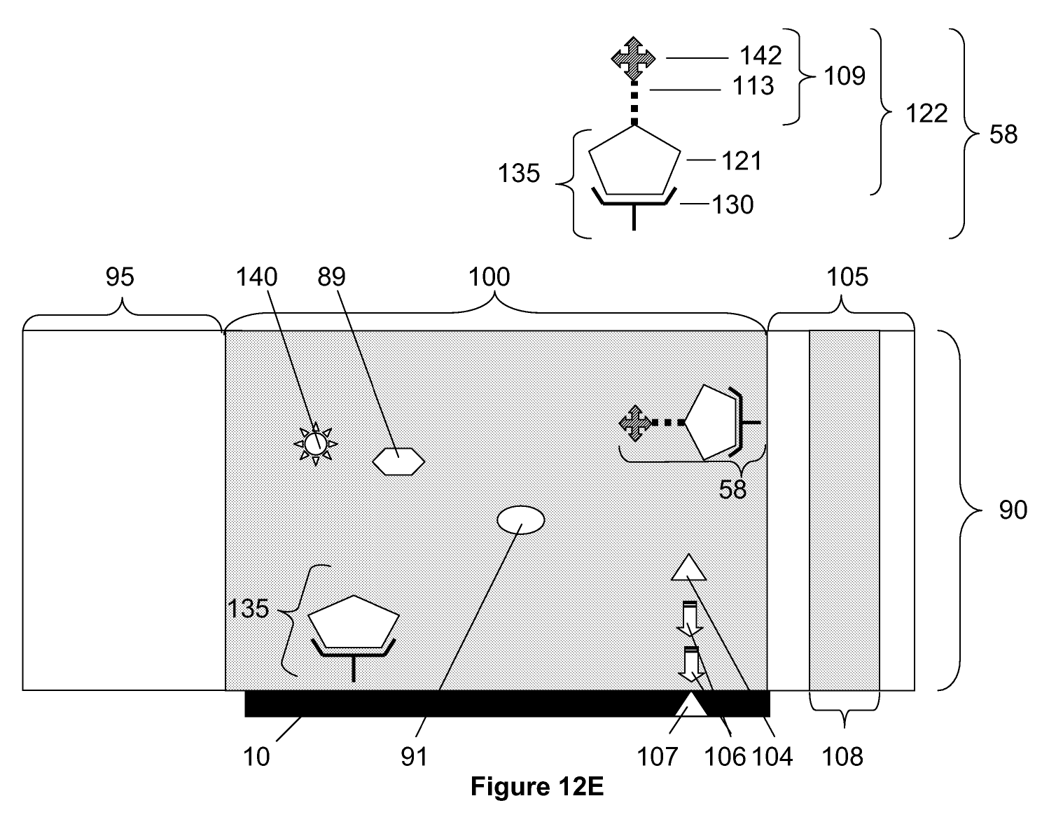

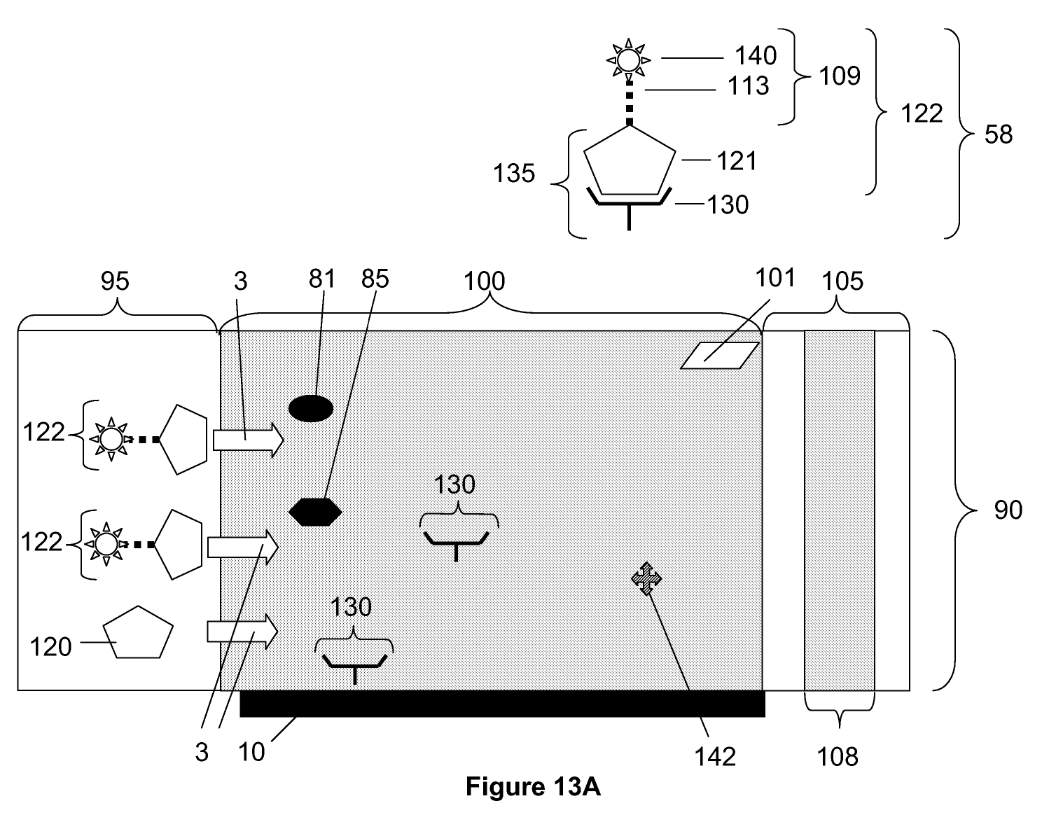

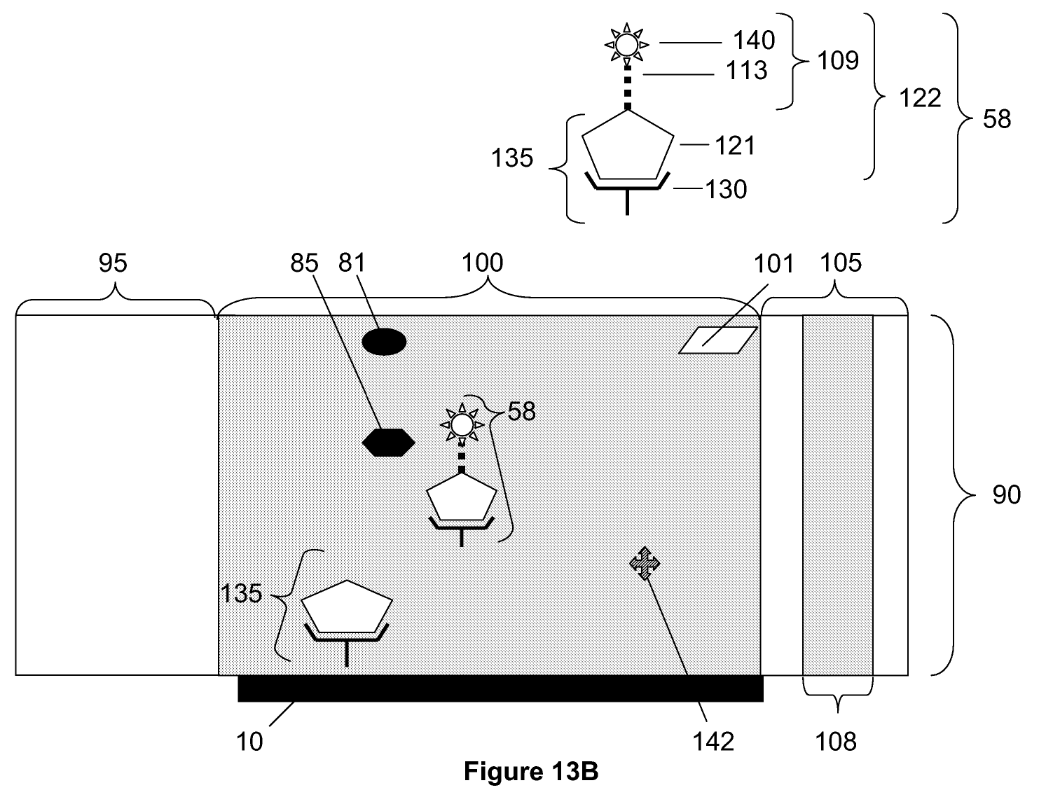

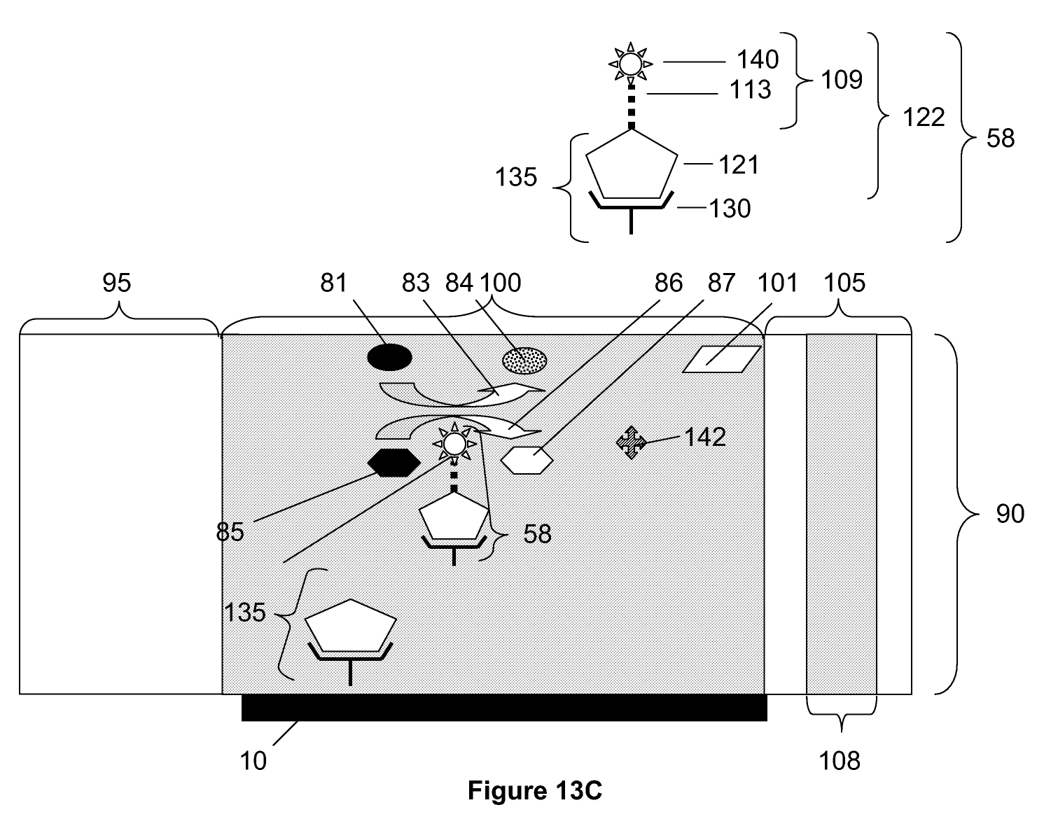

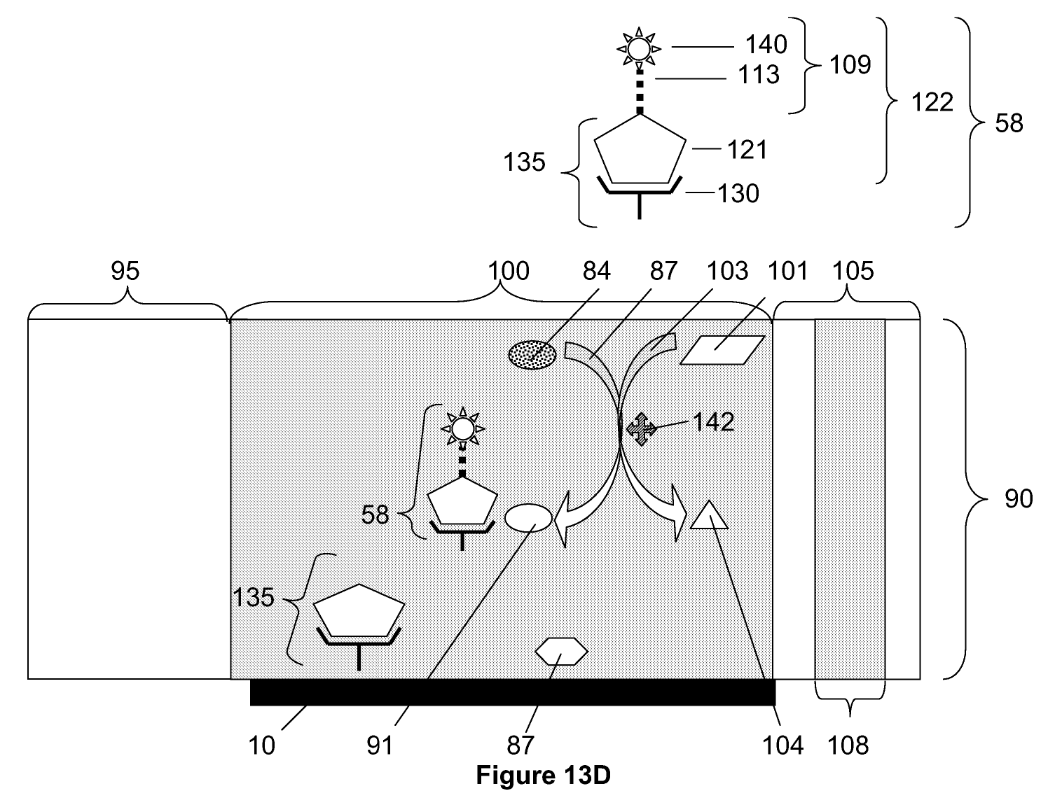

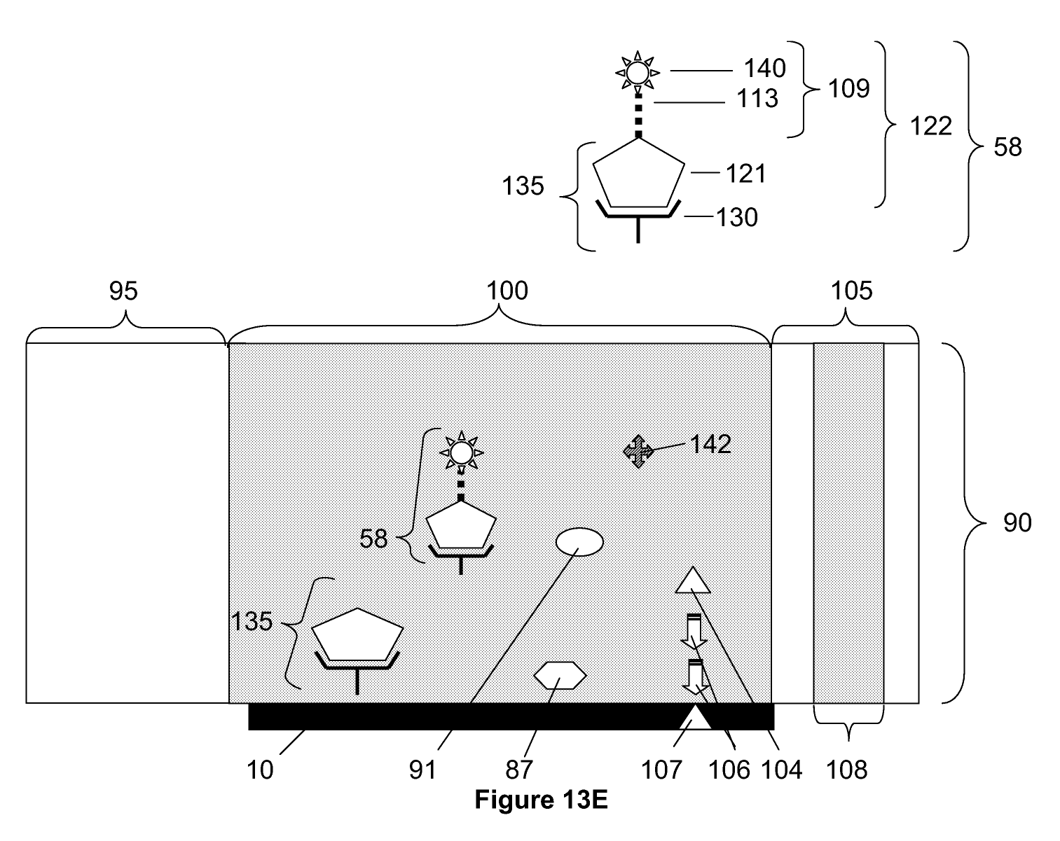

FIGS. 12A-E and FIGS. 13A-E are schematics illustrating embodiments of an immunoassay, comparable to FIGS. 6A-D and FIGS. 7-8, but illustrating some general features for a system that provides a two enzyme strategy in a competitive assay that may generate a chemical detection event in the device. These two embodiments are similar except that in FIGS. 12A-12E a first reduction-oxidation enzyme 140 in a two-enzyme catalytic series is not part of a labeled receptor 110 while a second reduction-oxidation enzyme 142 is part of a labeled receptor 110, and in FIGS. 13A-13E a first reduction-oxidation enzyme 140 in a two-enzyme catalytic series is part of a labeled receptor 110 while a second enzyme 142 is part of a labeled receptor 110.

As depicted in FIG. 12A, at the outset of the assay a detection zone 100 of a binder support medium 90 may contain binder 130, a first reduction-oxidation enzyme 140 and a dopant precursor compound 101. A first portion 95 of the binder support medium 90 contains a competitor complex 122 comprised of a second reduction-oxidation enzyme 142. The first portion 95 also contains a first chemical starting compound 81, second chemical starting compound 85; and analyte 120 from the applied sample, all of which may be carried into the detection zone 100 in the direction of fluid flow 3. As depicted in FIGS. 12B and 12C the analyte 120 and competitor complex 122 may be captured downstream to form a binder immobilized analyte 135 and a competitor-binder complex 58, respectively.

As depicted in FIG. 12C a first reduction-oxidation enzyme 140 may catalyze the transformation of the first chemical starting compound 81 and second chemical starting compound 85 to form a first chemical product 84 and second chemical product 87, respectively, by means of catalytic conversions 83 and 86, respectively. As depicted in FIG. 12D, a competitor-binder complex 58 that comprises a second reduction-oxidation enzyme 142 may then act upon the first chemical product 84 and dopant precursor 101 to produce a third chemical product 91 and dopant molecule 104, respectively, by means of conversions 89 and 103, respectively. As depicted in FIG. 12E, the dopant molecule 104 may undergo a diffusion 106 that transports it onto the surface of or into the mass of a conducting polymer component 10, resulting in a an embedded dopant 107. The embedded dopant 107 imparts electrical conductivity to the conducting polymer component 10.

As illustrated in FIGS. 13A-13E a different sequence of catalytic steps may be carried out for the two-enzyme competitive assay. As depicted in FIG. 13A, at the outset of the assay a first portion 95 of the binder support medium 90 may contain an analyte 120 from a liquid sample, and a competitor complex 122 comprised of a first reduction-oxidation enzyme 140; also at the outset a detection zone 100 of a binder support medium 90 may contain binder 130, a first chemical starting compound 81, second chemical starting compound 85, dopant precursor compound 101, and a second reduction-oxidation enzyme 142; and the analyte 120 and competitor complex 122 may be carried into the detection zone 100 in the direction of fluid flow 3. As depicted in FIGS. 13B the analyte 120 and competitor complex 122 may be captured downstream in the direction of fluid flow 3 to form binder immobilized analyte 135 and a competitor-binder complex 58, respectively.

As depicted in FIG. 13C a first reduction-oxidation enzyme 140 that forms part of a competitor complex 122 may catalyze the transformation of the first chemical starting compound 81 and second chemical starting compound 85 to form a first chemical product 84 and second chemical product 87, respectively, by means of catalytic conversions 83 and 86, respectively. As depicted in FIG. 13D, a second reduction-oxidation enzyme 142 may then act upon the first chemical product 84 and a dopant precursor 101 to produce a third chemical product 91 and dopant molecule 104, respectively, by means of conversions 89 and 103, respectively. As depicted in FIG. 13E, the dopant molecule 104 may undergo a diffusion 106 that transports it onto the surface of or into the mass of a conducting polymer component 10, resulting in an embedded dopant 107. The embedded dopant 107 imparts electrical conductivity to the conducting polymer component 10.

FIGS. 10A-10G, 11A-11E, 12A-12E, and 13A-13E also illustrates the use of an optional control zone 108 to confirm various results in a detection zone 100. Types of control zone strategies suitable for use in the above identified embodiments are not limited to the use of any particular control, and are similar to those discussed in FIGS. 5A-E and 6A-D.

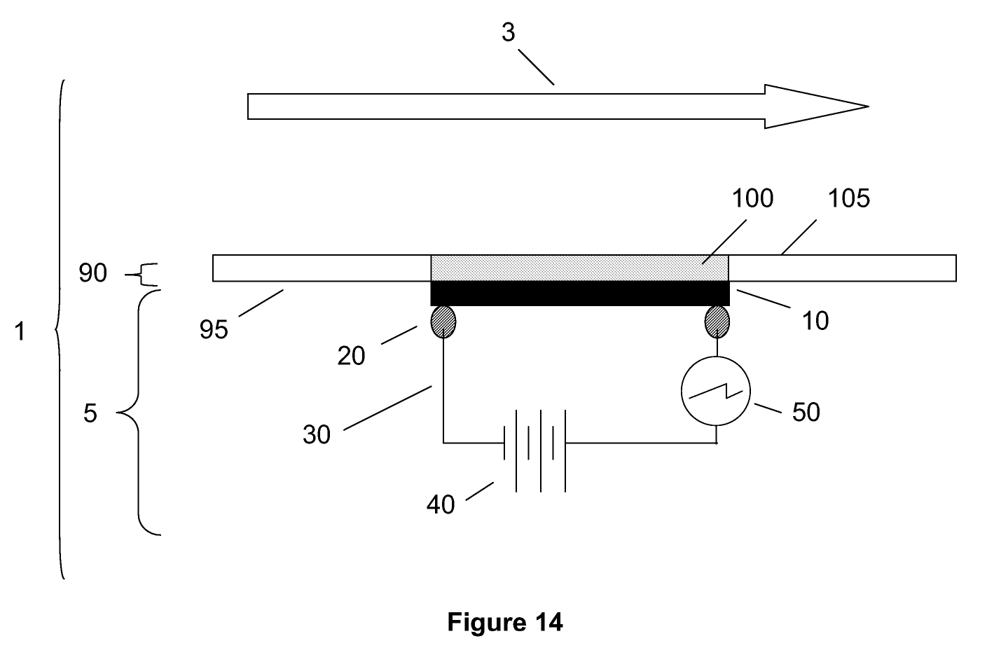

FIG. 14 is a schematic illustrating one embodiment of an immunoassay device 1; its elements are as described in FIG. 1. FIG. 14 further illustrates an embodiment of the invention in which all reagents and catalysts may be comprised in one or both of a first portion 95 or detection zone 100 of a binder support medium 90 without the aid of an accessory component or chemical depot such as a sample pad, tracer pad, reagent pad, sump, or other element in fluid communication with the binder support medium 90. Additionally FIG. 14 illustrates that a first portion 95 or detection zone 100 of a binder support medium 90 may receive a liquid sample and provide the necessary functions of the assay without the aid of an accessory component or chemical depot such as a sample pad, tracer pad, reagent pad, sump, or other element in fluid communication with the binder support medium 90.

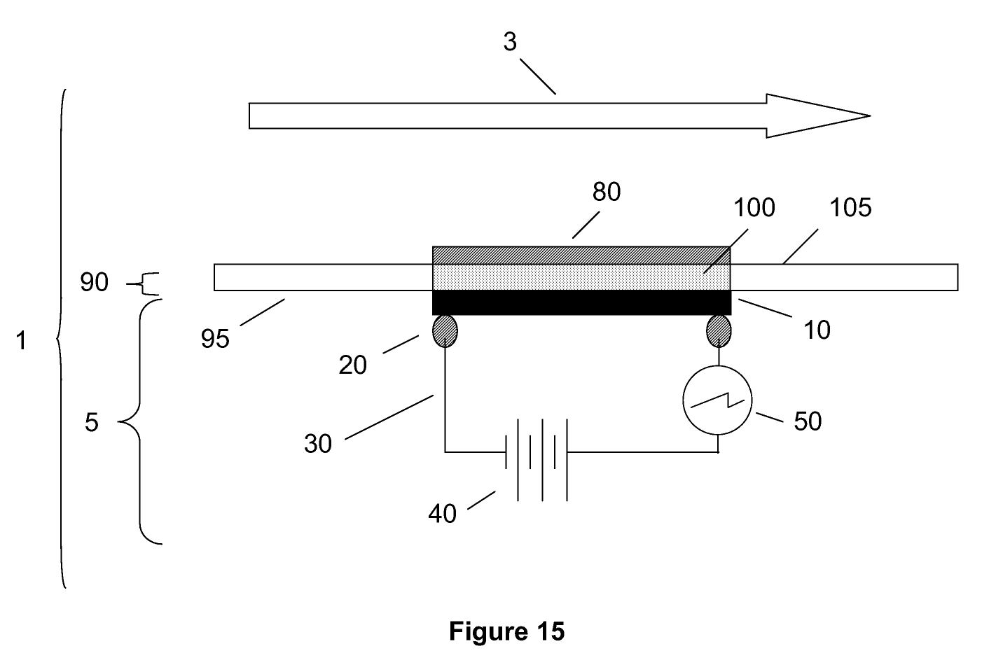

FIG. 15 is a schematic illustrating one embodiment of an immunoassay device 1 wherein a reagent pad 80 may be juxtaposed to or otherwise in fluid communication with a detection zone 100 of a binder support medium 90. The reagent pad 80 may comprise reagents, catalysts, labeled receptors, other chemical substances or physical features as described hereinabove. The reagent pad 80 may additionally be used to receive and optionally filter a liquid sample that may contain or lack an analyte.

The reagent pad 80 can be any porous material capable of dispensing a sample into liquid. The reagent pad 80 of the invention can be made from any hydrophilic porous or fibrous material capable of absorbing liquid rapidly. For example, porous plastics material, such as polypropylene, polyethylene, polyvinylidene fluoride, ethylene vinylacetate, acrylonitrile and polytetrafluoro-ethylene can be used as a sample pad 80. Materials such as cellulosic materials including nitro-cellulose, acrylic fibers such as non-woven spun-laced acrylic fiber (i.e., New Merge from DuPont) or HDK material (from HDK Industries, Inc.), glass, fiber, filter paper or pads, desiccated paper, paper pulp, fabric, and the like can also be used. The material selected for use as a sample pad 80 may also be chosen for its compatibility with the analyte 120 and assay reagents. For example, glass fiber filter paper may be utilized as a reagent pad 80 material for use in a human chorionic gonadotropin (hCG) assay device.

In certain instances, it may be advantageous to pre-treat the reagent pad 80 with a surface-active agent during manufacture in order to reduce any associated hydrophobicity, and enhance the ability of the reagent pad 80 to take up solution from and dispense into a liquid sample rapidly and efficiently. In one embodiment, the reagent pad 80 can be constructed from any material that may be capable of absorbing an aqueous solution. In another embodiment, the reagent pad 80 can be comprised of any material from which the liquid sample can flow through from the binder support medium 90. The material comprising the reagent pad 80 can be chosen such that the reagent pad 80 can be saturated with the liquid sample within a matter of seconds.

In additional embodiments, the functions of the reagent pad 80 may further include, for example: pH control/modification and/or specific gravity control/modification of the liquid sample applied, removal or alteration of components of the liquid sample which may interfere or cause non-specific binding in the assay, or to direct and control liquid sample flow through the binder medium 90.

In certain embodiments, the reagent pad 80 may include components that assist in the transfer of the reducing agents through the assay device. For example, when small quantities of viscous liquid sample are supplied from the reagent pad 80, it may be necessary to employ a wicking solution, preferably a buffered wicking solution, to carry the viscous liquid sample from the reagent pad 80 and through the assay device of the invention. When an aqueous liquid sample is used, a wicking solution generally is often not necessary but can be used to improve flow characteristics or adjust the pH of the liquid sample. The wicking solution can typically have a pH range from about 5.5 to about 10.5, and more particularly from about 6.5 to about 9.5. The pH may be selected to maintain a significant level of binding affinity between the specific binding members in a binding assay. However, the pH also may be selected to maintain significant enzyme activity for enzymes comprised within the assay. Illustrative buffers include but are not limited phosphate, carbonate, barbital, diethylamine, tris, 2-amino-2-methyl-1-propanol and the like. In one embodiment, the wicking solution can be contained on the reagent pad 80. In another embodiment, the wicking solution and the liquid sample can be combined prior to contacting the reagent pad 80. In an alternative embodiment, the wicking solution and the liquid sample can be applied sequentially to the reagent pad 80.

In another embodiment, the reagent pad 80 may also incorporate reagents useful to avoid cross-reactivity with non-target analytes that may exist in the liquid sample and/or to condition the liquid sample. These reagents may include, but are not limited to, for example, non-hCG blockers, anti-RBC reagents, Tris-based buffers, and EDTA, among others. When the use of whole blood is contemplated, anti-RBC reagents may be utilized. The reagent pad 80 may also incorporate other reagents such as ancillary specific binding members, liquid sample pretreatment reagents, and detection reagents.

In embodiments, the reagent pad 80 can be constructed to act as a filter for cellular components, hormones, particulate, and other certain substances that may be present in the liquid sample. The filtering aspect may allow an analyte of interest 120 to migrate through the device in a controlled fashion with fewer interfering substances than if the filtering aspect was not present. The filtering aspect can provide for a test having a higher probability of success and accuracy. The reagent pad 80 can further be modified by the addition of a filtration mechanism. The filtration mechanism can include any filter or trapping device used to remove particles above a certain size from the liquid sample. For example, the filtration mechanism can be used to remove red blood cells from a sample of whole blood, such that plasma is the fluid received by the binder medium 90.

In one embodiment, the reagent pad 80 may further comprise an analyte of interest, analyte homologue, or derivative thereof for use in a competitive assay.

Once in contact with the reagent pad 80, the liquid sample may thereafter permeate freely from the reagent pad 80 onto the vertically juxtaposed binder medium pad 90. The reagent pad 80 can be in direct fluid communication with the binder medium pad 90, such that the liquid sample can pass or migrate from the reagent pad 80 to the binder medium 90 via lateral or vertical flow. Fluid communication can include physical contact of the reagent pad 80 to the binder medium 90, as well as the separation of the reagent pad 80 from the binder medium 90 by an intervening space or additional material which still allows fluid communication between the reagent pad 80 and binder medium 90. In one embodiment, substantially all of the reagent pad 80 can overlap the binder medium 90 to enable the liquid sample to pass laterally or vertically through substantially any part of the reagent pad 80 to the binder medium 90.

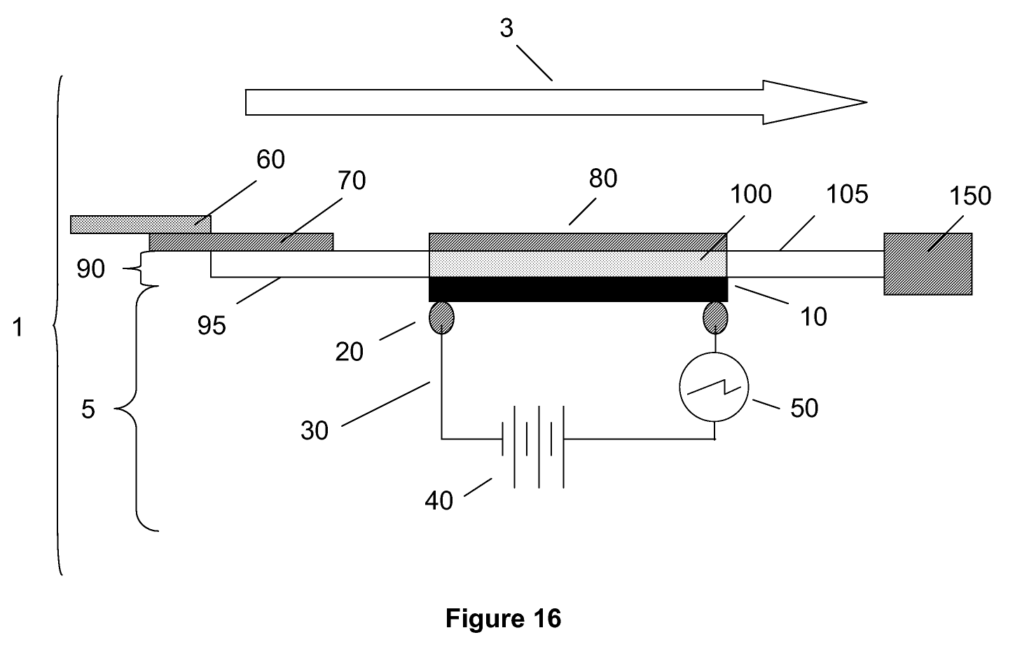

FIG. 16 is a schematic illustrating one embodiment of an immunoassay device 1 wherein a tracer pad 70 may be interposed between a sample pad 60 and a first portion 95 of a binder support medium 90, wherein the tracer pad 70 is in fluid communication with both the sample pad 60 and the first portion 95. A reagent pad 80 may be juxtaposed to or otherwise in fluid communication with a detection zone 100 of a binder support medium 90. And a sump 150 may be juxtaposed to or otherwise in fluid communication with a third portion 105 of a binder support medium 90, and downstream from a detection zone 100 of the support medium 90. The reagent pad 80 is as described in FIG. 13 above.

The sample pad 60 may be used to receive and optionally filter a liquid sample that may contain or lack an analyte. The sample pad 60 may additionally comprise reagents, catalysts, labeled receptors, other chemical substances or physical features as described hereinabove. A sample pad 60 utilized in the present invention can provide the receiving or collection point on the device for the application of a liquid sample. The sample pad 60 can be any porous material capable of receiving a liquid sample. The sample pad 60 of the present invention can be made from any hydrophilic porous or fibrous material capable of absorbing liquid rapidly. For example, porous plastics material, such as polypropylene, polyethylene, polyvinylidene fluoride, ethylene vinylacetate, acrylonitrile and polytetrafluoroethylene can be used as a sample pad 60. Materials such as cellulosic materials including nitro-cellulose, acrylic fibers such as non-woven spun-laced acrylic fiber (i.e., New Merge from DuPont) or HDK material (from HDK Industries, Inc.), glass, fiber, filter paper or pads, desiccated paper, paper pulp, fabric, and the like can also be used. The material selected for use as a sample pad 60 may also be chosen for its compatibility with the analyte and assay reagents. For example, glass fiber filter paper may be utilized as a sample pad 60 material for use in a human chorionic gonadotropin (hCG) assay device.

It certain instances, it may be advantageous to pre-treat these types of porous materials with a surface-active agent during manufacture in order to reduce any associated hydrophobicity, and enhance its ability to take up and deliver a liquid sample rapidly and efficiently. In one embodiment, the sample pad 60 may be constructed from any material that is capable of absorbing water. In another embodiment, the sample receiving zone may be comprised of any material from which the fluid sample can pass to the tracer pad 70. If desired the material comprising the sample pad 60 should be chosen such that the porous member can be saturated with aqueous liquid within a matter of seconds.

In additional embodiments, the functions of the sample pad 60 may further include, for example: pH control/modification and/or specific gravity control/modification of the liquid sample applied, removal or alteration of components of the liquid sample which may interfere or cause non-specific binding in the assay, or to direct and control sample flow to the tracer pad 70.

In certain embodiments, the sample pad 60 may include components that assist in the transfer of the liquid sample through the assay device. For example, when small quantities of non-aqueous or viscous test samples are applied to the sample pad 60, it may be necessary to employ a wicking solution, preferably a buffered wicking solution, to carry the test sample from the application pad and through the assay device of the present invention. When an aqueous test sample is used, a wicking solution generally is not necessary but can be used to improve flow characteristics or adjust the pH of the test sample. The wicking solution can typically have a pH range from about 5.5 to about 10.5, and more particularly from about 6.5 to about 9.5. The pH is selected to maintain a significant level of binding affinity between the specific binding members in a binding assay. The pH also must be selected to maintain significant enzyme activity in enzyme comprised within the sample pad 60 or within downstream members of the immunoassay device with which it is in fluid communication. Illustrative buffers include phosphate, carbonate, barbital, diethylamine, tris, 2-amino-2-methyl-1-propanol and the like. In one embodiment, the wicking solution is contained on the sample pad 60. In another embodiment, the wicking solution and the test sample are combined prior to contacting the sample pad 60. In an alternative embodiment, the wicking solution and the sample are applied sequentially to the sample pad 60. In one embodiment, the sample pad 60 further comprises an analyte of interest, analyte homologue, or derivative thereof for use in a competitive assay.

In another embodiment, the sample pad 60 may also incorporate reagents useful to avoid cross-reactivity with non-target analytes that may exist in the liquid sample and/or to condition the liquid sample. Depending on the particular embodiment, these reagents may include non-hCG blockers, anti-RBC reagents, Tris-based buffers, EDTA, among others. When the use of whole blood is contemplated, anti-RBC reagents are frequently utilized. In yet another embodiment, the sample pad 60 may incorporate other reagents such as ancillary specific binding members, liquid sample pretreatment reagents, and signal producing reagents.

In certain embodiments, the sample pad 60 can be constructed to act as a filter for cellular components, hormones, particulate, and other certain substances that may occur in the fluid sample. The filtering aspect allows an analyte of interest to migrate through the device in a controlled fashion with fewer, if any, interfering substances.

The filtering aspect, if present, often provides for a test having a higher probability of success and accuracy. In another preferred embodiment, the present invention can be further modified by the addition of a filtration means. The filtration means can be a separate material placed above the sample pad 60 or between the sample pad 60 and the tracer pad 70 material, or the material of the sample pad 60 itself can be chosen for its filtration capabilities. The filter can include any filter or trapping device used to remove particles above a certain size from the liquid sample. For example, the filter can be used to remove red blood cells from a sample of whole blood, such that plasma is the fluid received by the sample pad 60 and transferred to the tracer pad 70. In a further embodiment, the sample pad 60 is comprised of an additional sample application member (e.g., a wick). Thus, in one aspect, the sample pad 60 can comprise a sample pad 60 as well as a sample application member. Often the sample application member is comprised of a material that readily absorbs any of a variety of fluid samples contemplated herein, and remains robust in physical form. Frequently, the sample application member is comprised of a material such as white bonded polyester fiber. Moreover, the sample application member, if present, is positioned in fluid communication with a sample pad 60. This fluid communication can comprise an overlapping, abutting or interlaced type of contact. In occasional embodiments, the sample application member may be treated with a hydrophilic finishing. Often the sample application member, if present, may contain similar reagents and be comprised of similar materials to those utilized in sample pad 60.

Once introduced onto the sample pad 60, a liquid sample may thereafter permeate freely through or flow over the sample pad 60 onto the sequential layer in fluid communication. The tracer pad 70 may comprise reagents, catalysts, labeled receptors, other chemical substances or physical features as described hereinabove. In a more particular embodiment the tracer pad 70 comprises one or more labeled receptors in anhydrous form prior to the addition of liquid sample to the device.

The tracer pad 70 may be in laterally or vertically juxtaposed fluid flow communication with a sample pad 60 or otherwise in fluid flow contact with a sample pad 60. Liquid sample added to a sample pad 60 may flow through the sample pad and onto or through the tracer pad. The purpose of the tracer pad 70 is to provide mobile enzyme-labeled reagents that specifically interact with the analyte of interest, if present, in the sample. In one embodiment, the sample pad 60 is in direct vertical fluid flow contact with a tracer pad 70, such that the test sample can pass or migrate from the application pad to the tracer pad 70. Fluid flow contact can include physical contact of the sample pad 60 to the tracer pad 70, as well as the separation of the sample pad 60 from the tracer pad 70 by an intervening space or additional material which still allows fluid flow between the sample pad 60 and tracer pad 70. Substantially all of the sample pad 60 can overlap the tracer pad 70 to enable the liquid sample to pass through substantially any part of the sample pad 60 to the tracer pad 70.

The tracer pad 70 can be comprised of a porous material, such as, for example, high density polyethylene sheet material, non-woven spun-laced acrylic fiber, polyvinyl chloride, polyvinyl acetate, copolymers of vinyl acetate and vinyl chloride, polyamide, polycarbonate, polystyrene, untreated paper, cellulose blends, cellulose derivatives such as cellulose acetate and nitrocellulose, fiberglass, cloth including natural and synthetic cloths, polyester, an acrylonitrile copolymer, Rayon, glass fiber, porous gels such as silica gel, agarose, dextran, and gelatin; porous fibrous matrixes; starch based materials, such as Sephadex RTM-brand cross-linked dextran chains; ceramic materials; films of polyvinyl chloride and combinations of polyvinyl chloride-silica; and the like.

In certain embodiments, the tracer pad 70 material is treated with blocking and stabilizing agents. Blocking agents include bovine serum albumin (BSA), methylated BSA, casein, nonfat dry milk. Stabilizing agents are readily available and well known in the art, and may be used, for example, to stabilize labeled reagents. In frequent embodiments, employment of the selected blocking and stabilizing agents together with labeled reagent in the tracer pad followed by the drying of the blocking and stabilizing agents (e.g., a freeze-drying or forced air heat drying process) is utilized to attain improved performance of the assay configuration.

The tracer pad 70 generally contains a labeled receptor such as those shown in and described for FIGS. 5, 10, and 11. In one embodiment, the tracer pad 70 can have more than one labeled receptor specific for more than one specific analyte, wherein the labeled reagents have different detectable characteristics (e.g., different colors or different enzymatic activities) such that one analyte-labeled complex can be differentiated from another analyte-labeled complex in the detection zone 100.

The tracer pad 70 may also include at least one indicator serving as a control in the assay, such as one or more control labeled receptors. A control indicator can comprise detectible moieties that flow through the assay to a control zone by fluid sample flow through the device. In one embodiment, the detectible control moieties can be utilized to verify the conditions of the assay or the flow of the sample liquid. The visible moieties used in the control indicators may be the same or different color or enzymatic activities, or of the same or different type, as those used in the analyte-specific labeled reagents. If different colors are used, ease of observing the results may be enhanced.

The tracer pad 70 can also contain stabilizers, buffers, surfactants and other agents which improve the performance of the assay. The tracer pad 70 may include components that assist in the transfer of the liquid sample through the assay device. The functions of the tracer pad 70 may further include, for example: pH control/modification and/or specific gravity control/modification of the liquid sample applied, removal or alteration of components of the liquid sample which may interfere or cause non-specific binding in the assay, or to direct and control liquid sample flow to the first portion 95 of the binder support medium 90 including detection zone 100. Such additional reagents can be incorporated in the tracer pad or sample pad material. Alternatively, the developing reagents can be added to the sample before contact with the sample pad, or the sample pad can be exposed to the developing reagents after the binding reaction has taken place.

In one embodiment of the present invention, the use of a spacer or additional layer or layers of porous material placed between the tracer pad 70 and the binder support medium 90 is contemplated. Such an additional pad or layer can serve as a means to control the rate of flow of the test sample from the tracer pad 70 to the binder support medium 90. Such flow regulation is preferred when an extended incubation period is desired for the reaction of the sample and the reagent(s) in the tracer pad 70. Alternatively, such a layer can contain an additional assay reagent(s) which is preferably isolated from the tracer pad reagents until the liquid sample is added, or it can serve to prevent un-reacted assay reagents from passing to the binder support medium.

A sump 150 may be juxtaposed to or otherwise in fluid communication with a third portion 105 of a binder support medium 90, and downstream from a detection zone 100 of the support medium 90. The sump 150 absorbs liquid that makes its way to an end or edge of the binder support medium 90, and further drives the flow of liquid via capillary action in one direction to prevent backflow in the device. In one embodiment, the sump 150 is located in lateral juxtaposition to the binder support medium 90. In one embodiment, the liquid sample flows in a lateral unidirectional manner to the sump 150. In an alternative embodiment, the sump 150 is in lateral juxtaposition surrounding the binder support medium 90. In one embodiment, the liquid sample flows in a lateral multidirectional manner to the sump 150. In one embodiment, the liquid sample flows in a vertical direction to the sump 150. In one embodiment the liquid sample flows by non-bibulous flow to the sump 150. Examples of suitable absorbent materials for use in a sump 150 include cotton fiber, tissue, and absorbent paper such as Whatman paper. Other examples of suitable absorbent materials capable of acting as a sump 150 are generally known by one of ordinary skill in the art. The absorption and capacity of the sump 150 may be chosen to absorb substantially all of the fluid flow through the device to its full extent approximately instantaneously upon contact with the liquid sample. Alternatively the sump 150 may be selected such that a liquid sample is absorbed by the sump 150 more slowly and or only incompletely, thereby slowing the rate of wicking through the binder support medium 90 to a desired flow rate and optionally saturating the sump 150 before the binder support medium 90 is entirely divested of flowable liquid sample.

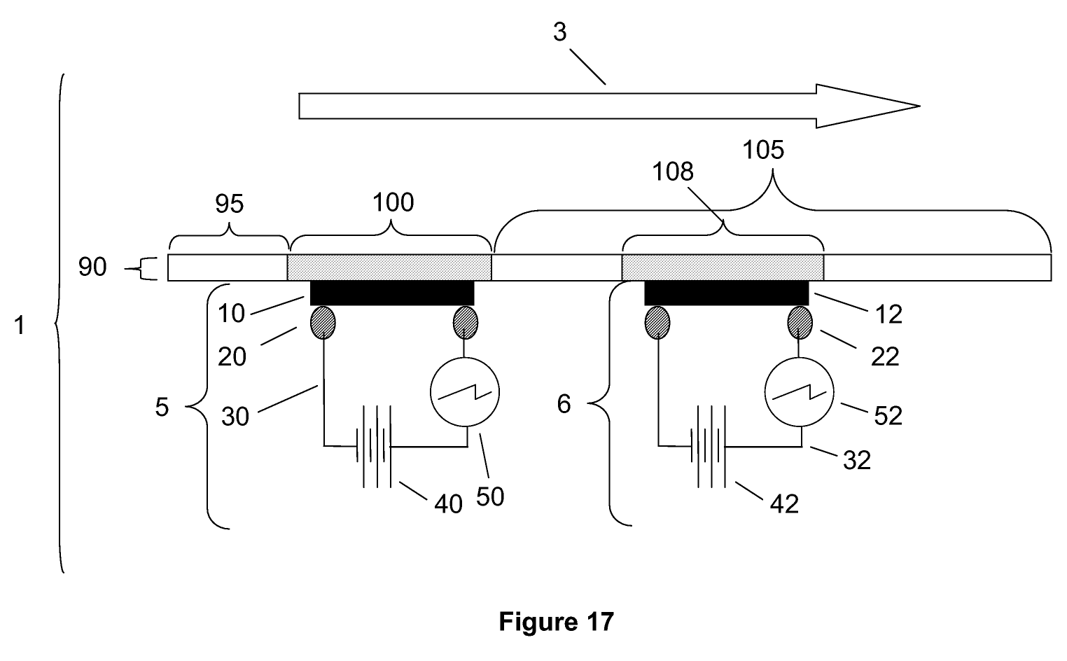

FIG. 17 is a schematic illustrating one embodiment of an immunoassay device 1 wherein the device 1 is configured as in FIGS. 1 and 14, wherein a second conducting polymer component 12, in electrical communication with a second electrical configuration 6 that comprises a second set of electrical leads 22, second circuit 32, second voltage source 42, and second readout mechanism 52. In certain embodiments, one or more elements of the electrical configuration 5 and second electrical configuration 6 may be used in common, and that these two configurations may be component parts of a more extensive single circuit.

In one embodiment, the second electrical configuration 6 can be in fluid communication with a control zone 108, allowing the second electrical configuration to detect the generation of a dopant molecule in the presence of a specified control reagent. For example, a control zone 108 may comprise immobilized analyte to capture excess labeled receptor that has not bound to an analyte upstream; the captured labeled receptor 110 in the control zone 108 in combination with substrate reagents can generate a dopant that can be detected through interactions with the second electrical configuration 6, and indicated to a second readout mechanism 52.

In one embodiment the control zone 108 may host the complexation of a dopant precursor compound and first chemical product, such that uncomplexed labeled receptor or unbound competitor complex from upstream could react and be quantified in the control zone 108. Thus the second conducting polymer component 12 in fluid communication with the control zone 108 could serve as a second readout circuit 6 to confirm results of a first readout circuit 5 that detects dopant generation in the detection zone 100.

In an alternative embodiment, the second conducting polymer component 12 may be in fluid communication with a second detection zone, wherein the second detection zone is directed to the determination of the presence of a second analyte of interest.

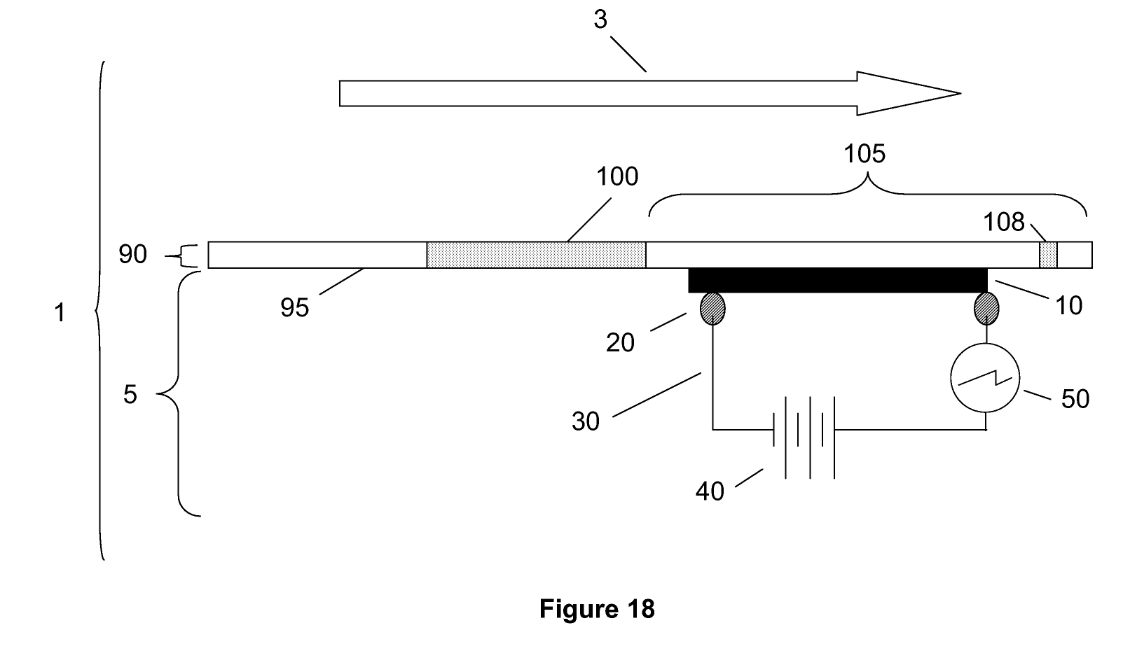

FIG. 18 is a schematic illustrating one embodiment of an immunoassay device 1 wherein the device 1 is configured as in FIGS. 1 and 12, except that the conducting polymer component 10 is juxtaposed to or otherwise in fluid communication with a third portion 105 of a binder support medium 90, and the arrangement of the corresponding readout circuit 5 accommodates that location of the conducting polymer component 10.

A benefit of placing conducting polymer components downstream from the detection zone is that dopant molecules that are washed downstream from the detection zone 100 by fluid flow may be detected by the conducting polymer component 10 whereas they might otherwise have passed beyond it without detection.

Housing

If the immunoassay device is contained in a housing, apertures can be contained in the housing. The apertures can be located relative to the detection and any control zones on the binder support membrane, wherein the position of the aperture allows a user, or detection device, visual access to the detection zone(s) or control zone(s). In certain embodiments, the apertures for visual access to detection zone and control zone are located on the bottom of the housing. In an alternative embodiment, the apertures are located on the top or side of the housing. The housing also can include an aperture for accessing the sample pad, tracer pad, or reagent pad, where applicable, in order to apply the liquid sample thereto.

Kits

The invention further provides kits for carrying out immunoassays utilizing the immunoassay device as described herein. The kits can include buffers, reagents, instructions on use, or other information or compounds that are helpful in conducting an assay utilizing the immunoassay device. For example, a kit according to the invention can comprise the assay device with its incorporated reagents as well as a wicking solution and/or test sample pretreatment reagents. Other assay components known to one of ordinary skill in the art, such as buffers, stabilizers, detergents, bacteria inhibiting agents and the like can also be present in the kit. In addition, the kit can contain packaging materials and directions on how to use the device. In another aspect of the present invention, kits are provided comprising the immunoassay device 1 in which at least one member of the device requires further assembly.

It should be understood that the foregoing relates only to illustrate the embodiments of the invention, and that numerous changes may be made therein without departing from the scope and spirit of the invention.

What is claimed is:

1. A device comprising:
a) a binder support medium comprising at least one detection zone for the detection of an analyte in a liquid sample;
b) at least one label moiety, wherein the label is capable of participating in the generation of a dopant;
c) a conducting polymer in fluid communication with the binder support medium, the conducting polymer capable of having its electrical conductivity changed after being doped with the dopant;
d) a circuit that comprises a voltage source, wherein an electric potential may be maintained across at least one portion of one dimension of the conducting polymer by means of the circuit.

2. The device of claim 1, wherein the electrical conductivity of the conducting polymer increases after being doped with the dopant.

3. The device of claim 1, wherein the circuit further comprises a readout mechanism capable of registering an indication of the magnitude of a measured electrical property in response to an electrical current that is conducted through the conducting polymer.

4. The device according to claim 1, wherein the label moiety comprises a first reduction-oxidation enzyme.

5. The device according to claim 4, wherein the device further comprises at least one reducing agent.

6. The device according to claim 5, wherein the device further comprises at least one precursor molecule capable of conversion to the dopant upon interaction with the reduction-oxidation enzyme and the reducing agent.

7. The device according to claim 5, wherein the device further comprises a second reduction-oxidation enzyme.

8. The device according to claim 6, wherein the precursor molecule is located within the detection zone of the binder support medium.

9. The device according to claim 4, wherein the reduction-oxidation enzyme is selected from the group consisting of lactose peroxidase and glucose oxidase.

10. The device according to claim 1, wherein the dopant is selected from the group consisting of iodine and iodide.

11. The device according to claim 1, wherein the binder support medium is in fluid communication with one or more elements selected from the group consisting of a sample pad, a tracer pad, a reagent pad, and a sump.

12. A device comprising:
a) a binder support medium comprising at least one detection zone for the detection of an analyte in a liquid sample;
b) at least one chemical compound that is in fluid communication with the binder support medium, and which is capable of being transformed into a dopant or a dopant precursor compound by a reduction-oxidation enzyme;
c) at least one label moiety, wherein the label moiety is a reduction-oxidation enzyme capable of participating in the generation of the dopant;
d) a conducting polymer component in fluid communication with the binder support medium, the conducting polymer component capable of having its electrical conductivity changed after being doped with the dopant; and,
e) a circuit that comprises a voltage source, wherein an electric potential may be maintained across at least one portion of one dimension of the conducting polymer by means of the circuit.

13. The device of claim 12, wherein the electrical conductivity of the conducting polymer increases after being doped with the dopant.

14. The device of claim 12, wherein the circuit further comprises a readout mechanism capable of registering an indication of the magnitude of a measured electrical property in response to an electrical current.

15. The device according to claim 12, wherein the label moiety is selected from the group consisting of glucose oxidase and lactose peroxidase.

16. The device according to claim 12, wherein the device further comprises additional reagents selected from the group consisting of reducing agents and dopant precursor compounds, or combinations thereof.

17. The device according to claim 12, wherein the device further comprises a second reduction-oxidation enzyme.

18. The device according to claim 12, wherein the binder support medium is in fluid communication with one or more elements selected from the group consisting of a sample pad, a tracer pad, a reagent pad, and a sump.

19. A method for detecting the presence or amount of an analyte in a liquid sample comprising:
a) applying a liquid sample suspected of containing an analyte to a device;
b) contacting the analyte, if present, with a labeled receptor molecule, wherein the label is a reduction-oxidation enzyme, to form an analyte-labeled receptor complex;
c) transporting the analyte-labeled receptor complex through a binder support medium by means of liquid flow to a detection zone portion of the binder support medium;
d) immobilizing the analyte-labeled receptor complex in the detection zone portion of the binder support medium;
e) removing by liquid flow any reduction-oxidation enzyme that is not immobilized in the detection zone portion of the binder support medium, wherein the removal by liquid flow may occur prior to or concurrent with any of the events below;
f) transforming at least one chemical compound by the reduction-oxidation enzyme portion of the immobilized analyte-labeled receptor complex to form a dopant;
g) diffusing the dopant into or onto the surface of a conducting polymer that is in fluid communication with the detection zone portion, to form a doped conducting polymer; and,
h) detecting the degree of doping of the conducting polymer by applying an electrical potential across the conducting polymer, to determine the presence, the amount, or the presence and amount of the analyte.

20. The method of claim 19, wherein the analyte portion of the analyte-labeled receptor complex binds to a binder by means of a ligand-receptor interaction.

21. The method of claim 19, wherein the reduction oxidation enzyme is selected from the group consisting of lactose peroxidase and glucose oxidase.

22. The method of claim 19, wherein the dopant generated is iodine or iodide, or a derivative thereof.

23. A method for detecting the presence or amount of an analyte in a liquid sample comprising:
a) applying a liquid sample suspected of containing an analyte to a device;
b) contacting the analyte, if present, with a labeled receptor molecule, wherein the label is a first reduction-oxidation enzyme, to form an analyte-labeled receptor complex;
c) transporting the analyte-labeled receptor complex through a binder support medium by means of liquid flow to a detection zone portion of the binder support medium;
d) immobilizing the analyte-labeled receptor complex in the detection zone portion of the binder support medium;
e) removing by liquid flow any first reduction-oxidation enzyme that is not immobilized in the detection zone portion of the binder support medium, wherein the removal by liquid flow may occur prior to or concurrent with any of the events below;
f) transforming at least one chemical compound with the first reduction-oxidation enzyme portion of the immobilized analyte-labeled receptor complex, to form a dopant precursor molecule;
g) transforming the dopant precursor molecule to a dopant with a second reduction-oxidation enzyme that is present within the detection zone portion;
h) diffusing the dopant into or onto the surface of a conducting polymer that is in fluid communication with the detection zone portion, to form a doped conducting polymer; and
i) detecting the degree of doping of the conducting polymer by applying an electrical potential across the conducting polymer, to determine the presence, the amount, or both the presence and amount of the analyte.

24. The method of claim 23, wherein the analyte portion of the analyte-labeled receptor complex binds to a binder by means of a ligand-receptor interaction.

25. The method according to claim 23, wherein the first reduction-oxidation enzyme is glucose oxidase.

26. The method according to claim 23, wherein the second reduction-oxidation enzyme is lactose peroxidase.

27. A method for detecting the presence or amount of an analyte in a liquid sample comprising the steps of:
a) applying a liquid sample containing or lacking an analyte to a device, wherein the device comprises a competitor bound to a label moiety forming a competitor complex, the competitor being an analyte, analogue, or derivative thereof, and the label moiety being a reduction-oxidation enzyme;
b) transporting the competitor complex and analyte contained in the liquid sample, if present, through a binder support medium by means of liquid flow to a detection zone portion of the binder support medium;
c) immobilizing the analyte in the detection zone whereby the competitor portion of the competitor complex and the analyte contained in the liquid sample compete for the binding of the binder in the detection zone by means of a ligand-receptor interaction;
d) removing by liquid flow any competitor complex that is not immobilized in the detection zone, wherein the removal by liquid flow may occur prior to or concurrent with any of the events below;
e) transforming at least one chemical compound with the reduction-oxidation enzyme portion of the immobilized competitor complex to form a dopant;
f) diffusing the dopant into or onto the surface of a conducting polymer that is in fluid communication with the detection zone portion, to form a doped conducting polymer; and
g) detecting the degree of doping of the conducting polymer by applying an electrical potential across the conducting polymer, to determine the amount, the presence or the presence and amount of the analyte.

28. The method of claim 27, wherein the reduction oxidation enzyme is selected from the group consisting of lactose peroxidase and glucose oxidase.

29. The method of claim 27, wherein the dopant generated is iodine or iodide, or a derivative thereof.

30. A method for detecting the presence or amount of an analyte in a liquid sample comprising:
a) applying a liquid sample containing or lacking an analyte to a device, wherein the device contains a competitor bound to a label forming a competitor complex, the competitor being an analyte, analogue, or derivative thereof, and the label being a reduction-oxidation enzyme;
b) transporting the competitor complex and analyte contained in the liquid sample, if present, through a binder support medium by means of liquid flow to a detection zone portion of the binder support medium;
c) immobilizing the analyte in the detection zone whereby the competitor portion of the competitor complex and the analyte contained in the liquid sample compete for the binding of the binder in the detection zone by means of a ligand-receptor interaction;
d) removing by liquid flow any competitor complex that is not immobilized in the detection zone, wherein the removal by liquid flow may occur prior to or concurrent with any of the events below;
e) transforming at least one chemical compound with the reduction-oxidation enzyme portion of the immobilized competitor complex to form a dopant precursor molecule;
f) transforming the dopant precursor molecule to a dopant with a second reduction-oxidation enzyme that is present within the detection zone portion;
g) diffusing the dopant into or onto the surface of a conducting polymer that is in fluid communication with the detection zone portion, to form a doped conducting polymer; and
h) detecting the degree of doping of the conducting polymer by applying an electrical potential across the conducting polymer component, to determine the presence, the amount or the presence and amount of the analyte.

31. The method according to claim 30, wherein the first reduction-oxidation enzyme is glucose oxidase.

32. The method according to claim 30, wherein the second reduction-oxidation enzyme is lactose peroxidase.
